(19) United States
(12) Patent Application Publication
(10) Pub. No.: US 2024/0099989 A1
(43) Pub. Date: Mar. 28, 2024

(54) AGENT FOR PREVENTING OR IMPROVING PERIPHERAL NEUROPATHY

(71) Applicants: KINKI UNIVERSITY, Osaka (JP); KOBAYASHI PHARMACEUTICAL CO., LTD., Osaka (JP)

(72) Inventors: Shozo NISHIDA, Osaka (JP); Masanobu TSUBAKI, Osaka (JP); Tomoya TAKEDA, Osaka (JP); Toshio MORIKAWA, Osaka (JP); Shota KAJIYAMA, Osaka (JP); Satoru ISHIKAWA, Osaka (JP)

(73) Assignees: KINKI UNIVERSITY, Osaka (JP); KOBAYASHI PHARMACEUTICAL CO., LTD., Osaka (JP)

(21) Appl. No.: 18/275,521

(22) PCT Filed: Feb. 7, 2022
(86) PCT No.: PCT/JP2022/004603

§ 371 Date: Aug. 2, 2023

(30) Foreign Application Priority Data
Feb. 5, 2021 (JP) 2021-017510

(51) Int. Cl. A61K 31/047; A61P 25/02

(52) U.S. Cl. CPC A61K 31/047; A61P 25/02

(57) ABSTRACT

There are no drugs or food medicines that can prevent or ameliorate, among side effects caused by drugs, particularly peripheral neuropathy that is caused by anticancer drugs such as oxaliplatin.

An agent for preventing or ameliorating peripheral neuropathy including at least one selected from xylitol, L-talitol, and D-threitol as an active ingredient can ameliorate tingling limbs, limb pain, decreased deep tendon reflexes, muscle weakness, allodynia, hyperalgesia, hand fine motor skill disability, gait disturbance, stumbling, falling, flexion impairment (difficulty or inability to sit on one's knees, cross-legged, sideways, in a chair, etc.), limb paralysis, or the like, induced by drugs such as anticancer drugs or diabetes.

### TECHNICAL FIELD

The present invention relates to an agent for preventing or ameliorating peripheral neuropathy, in particular, an agent for preventing or ameliorating peripheral neuropathy that can be suitably used for preventing or ameliorating the peripheral neuropathy that is caused by anticancer drugs or diabetes.

### BACKGROUND ART

As drugs used in chemotherapy for malignant tumors, those with various mechanisms of action have been developed. These drugs inhibit tumor cell survival or proliferation on the basis of their specific mechanisms of action. However, these drugs generally act not only on tumor cells, but also on normal cells in the same manner. Thus, administration of drugs used in chemotherapy causes side effects such as peripheral neuropathy, hair loss, vomiting, gastrointestinal disturbance, hepatotoxicity, nephrotoxicity, and neurotoxicity, along with the tumor-inhibitory effects of the drugs.

Among these side effects, peripheral neuropathy causes sensory hypersensitivity (allodynia), a type of pain triggered by a stimulus that would not cause pain to a healthy person. A tingling or prickling feeling associated with this sensory hypersensitivity continues for a long period of time, often leading to discontinuation of chemotherapy. Thus, this side effect is regarded as a major problem in the field of chemotherapy.

Conventionally, for peripheral neuropathy that develops as a side effect of chemotherapy, a pain reliever such as gabapentin or ketamine, an antiepileptic drug such as lamotrigine or clonazepam, an antidepressant such as clomipramine or duloxetine, a Kampo medicine such as Goshajinkigan or Shakuyakukanzoto, or a vitamin B preparation has been administered. However, none can be said to be achieving great effects.

Under such circumstances, some drugs have been proposed to ameliorate this peripheral neuropathy. Patent Literature 1 proposes a composition containing amino acids including serine and lipids including an n-3 fatty acid.

Furthermore, Patent Literature 2 discloses that a specific cyclic amine compound can function as a therapeutic or preventive agent for peripheral neuropathy.

Furthermore, it is known that peripheral neuropathy with similar symptoms develops as a symptom of diabetes. This peripheral neuropathy impairs the patient's quality of life, thus eliciting a strong demand for ameliorating the symptoms of diabetic peripheral neuropathy.

Note that Patent Literature 3 describes that a lactam compound is effective as an agent for enhancing sugar transport and can be used as a preventive and/or therapeutic agent for diabetes, diabetic peripheral neuropathy, diabetic nephropathy, diabetic microangiopathy, impaired glucose tolerance, or obesity.

### CITATION LIST

### Patent Literature

PTL 1: Japanese Patent Application Laid-Open No. 2019-182881
PTL 2: International Publication No. 2018/181860
PTL 3: Japanese Patent Application Laid-Open No. 2006-213732

### SUMMARY OF INVENTION

### Technical Problem

The composition in Patent Literature 1 is composed of amino acids and fatty acids and is likely to be relatively safe for the human body based on its track record. However, it is also considered that a peptide in the composition requires further studies to optimize its pharmacokinetic properties. The agent in Patent Literature 2 is a non-peptide agent. Thus, it is necessary to examine side effects of the agent itself.

Patent Literature 3 discloses, also in examples, that the lactam compound has the ability to transport sugars. However, Patent Literature 3 only discloses that the lactam compound has an effect of lowering a blood sugar level without any specific description regarding peripheral neuropathy. The cause of diabetic peripheral neuropathy is still unclear. Furthermore, diabetic peripheral neuropathy is a complication arising from diabetes, and it is more important to control the progression of diabetes.

### Solution to Problem

The present invention has been conceived in view of the above-mentioned problems and provides an agent for preventing or ameliorating peripheral neuropathy using an ingredient, other than a peptide, that has few side effects. In particular, the agent for preventing or ameliorating peripheral neuropathy according to the present invention is effective for both chemotherapy-induced peripheral neuropathy and diabetic peripheral neuropathy.

More specifically, the agent for preventing or ameliorating peripheral neuropathy according to the present invention is characterized by including at least one selected from xylitol, L-talitol, and D-threitol.

That is, the present invention provides the invention in the following aspects.
Item 1. An agent for preventing or ameliorating peripheral neuropathy characterized by including at least one selected from xylitol, L-talitol, and D-threitol.
Item 2. The agent for preventing or ameliorating peripheral neuropathy according to item 1, in which the peripheral neuropathy is induced by administration of an anticancer drug.
Item 3. The agent for preventing or ameliorating peripheral neuropathy according to item 2, in which the anticancer drug is a platinum-based anticancer drug, a microtubule polymerization stabilizer, a microtubule polymerization inhibitor, a proteasome inhibitor, or the like.
Item 4. The agent for preventing or ameliorating peripheral neuropathy according to item 1, in which the peripheral neuropathy is diabetic peripheral neuropathy.
Item 5. The agent for preventing or ameliorating peripheral neuropathy according to any of items 1 to 4, in which the agent is a pharmaceutical product.
Item 6. The agent for preventing or ameliorating peripheral neuropathy according to any of items 1 to 4, in which the agent is a food product.
Item 7. A method for preventing or ameliorating peripheral neuropathy, including a step of administering at least one selected from xylitol, L-talitol, and D-threitol.
Item 8. A use of xylitol, L-talitol, or D-threitol in a production of an agent for preventing or ameliorating peripheral neuropathy, the agent being used for preventing or ameliorating the peripheral neuropathy.
Item 9. Xylitol, L-talitol, or D-threitol used for preventing or ameliorating peripheral neuropathy.
Item 10. A peripheral nerve outgrowth inhibition suppressant characterized by including at least one selected from xylitol, L-talitol, and D-threitol.
Item 11. A method for suppressing peripheral nerve outgrowth inhibition, including a step of administering at least one selected from xylitol, L-talitol, and D-threitol.
Item 12. A use of xylitol, L-talitol, or D-threitol in a production of a peripheral nerve outgrowth inhibition suppressant, the suppressant being used for suppressing peripheral nerve growth inhibition.
Item 13. Xylitol, L-talitol, or D-threitol used for suppressing peripheral nerve growth inhibition.

### Advantageous Effects of Invention

The present invention can provide the agent for preventing or ameliorating peripheral neuropathy. That is, administration or ingestion of at least one selected from xylitol, L-talitol, and D-threitol can ameliorate limb tingling, limb pain, decreased deep tendon reflexes, muscle weakness, allodynia, hyperalgesia, hand fine motor skill disability, gait disturbance, stumbling, falling, flexion impairment (difficulty or inability to sit on one's knees, cross-legged, sideways, in a chair, etc.), limb paralysis, or the like that are induced by cancer chemotherapy or diabetes. Furthermore, the preventive or ameliorative agent according to the present invention can also be used for the prevention of the above-mentioned peripheral neuropathy by taking the agent at the same time as the start of chemotherapy or after noticing that excess carbohydrates have been consumed.

It has been necessary to reduce the dose of anticancer drugs or suspend cancer chemotherapy to deal with peripheral neuropathy. However, using the agent of the present invention allows appropriate cancer treatments to continue, leading to a quicker recovery from cancer.

Furthermore, the present invention provides the agent for preventing or ameliorating peripheral neuropathy that can be easily administered or ingested at home. Provision of such an agent is very helpful for patients undergoing cancer treatments at home. Furthermore, the quality of life (QOL) of the patients is improved by preventing or ameliorating peripheral neuropathy that is caused by cancer chemotherapy or diabetes.

Furthermore, as is well known, sugar alcohols such as xylitol, L-talitol, and D-threitol are safe for the human body. It has been demonstrated that, except diarrhea, side effects due to an overdose of sugar alcohols have not been observed.

Note that peripheral neuropathy is caused not only by cancer chemotherapy or diabetes, but also by administration of other drugs, trauma, infection, or the like. Using the preventive or ameliorative agent according to the present invention can prevent or ameliorate symptoms caused by these types of peripheral neuropathy.

### BRIEF DESCRIPTION OF DRAWINGS

FIG. 1 is a diagram showing the results of a cold plate test in which xylitol was administered to the mice together with oxaliplatin.

FIG. 2 is a diagram showing the results of a von Frey test in which xylitol was administered to the mice together with oxaliplatin.

FIG. 3 is a diagram showing the results of a cold plate test in which xylitol was administered to the mice together with paclitaxel.

FIG. 4 is a diagram showing the results of a von Frey test in which xylitol was administered to the mice together with paclitaxel.

FIG. 5 is a diagram showing the results of a cold plate test in which xylitol was administered to the mice together with vincristine.

FIG. 6 is a diagram showing the results of a von Frey test in which xylitol was administered to the mice together with vincristine.

FIG. 7 is a diagram showing the results of a cold plate test in which xylitol was administered to the mice together with bortezomib.

FIG. 8 is a diagram showing the results of a von Frey test in which xylitol was administered to the mice together with bortezomib.

FIG. 9 is a diagram showing the results of a cold plate test in which xylitol was administered to the mice that had developed peripheral neuropathy induced by oxaliplatin.

FIG. 10 is a diagram showing the results of a von Frey test in which xylitol was administered to the mice that had developed peripheral neuropathy induced by oxaliplatin.

FIG. 11 is a diagram showing the results of a cold plate test in which xylitol was administered to the mice that had developed peripheral neuropathy induced by paclitaxel.

FIG. 12 is a diagram showing the results of a von Frey test in which xylitol was administered to the mice that had developed peripheral neuropathy induced by paclitaxel.

FIG. 13 is a diagram showing the results of a cold plate test in which xylitol was administered to the mice that had developed peripheral neuropathy induced by vincristine.

FIG. 14 is a diagram showing the results of a von Frey test in which xylitol was administered to the mice that had developed peripheral neuropathy induced by vincristine.

FIG. 15 is a diagram showing the results of a cold plate test in which xylitol was administered to the mice that had developed peripheral neuropathy induced by bortezomib.

FIG. 16 is a diagram showing the results of a von Frey test in which xylitol was administered to the mice that had developed peripheral neuropathy induced by bortezomib.

FIG. 17 is a diagram showing the results of a cold plate test in which D-threitol was administered to the mice together with oxaliplatin.

FIG. 18 is a diagram showing the results of a von Frey test in which D-threitol was administered to the mice together with oxaliplatin.

FIG. 19 is a diagram showing the results of a cold plate test in which D-threitol was administered to the mice together with paclitaxel.

FIG. 20 is a diagram showing the results of a von Frey test in which D-threitol was administered to the mice together with paclitaxel.

FIG. 21 is a diagram showing the results of a cold plate test in which D-threitol was administered to the mice together with vincristine.

FIG. 22 is a diagram showing the results of a von Frey test in which D-threitol was administered to the mice together with vincristine.

FIG. 23 is a diagram showing the results of a cold plate test in which D-threitol was administered to the mice together with bortezomib.

FIG. 24 is a diagram showing the results of a von Frey test in which D-threitol was administered to the mice together with bortezomib.

FIG. 25 is a diagram showing the results of a cold plate test in which D-threitol was administered to the mice that had developed peripheral neuropathy induced by oxaliplatin.

FIG. 26 is a diagram showing the results of a von Frey test in which D-threitol was administered to the mice that had developed peripheral neuropathy induced by oxaliplatin.

FIG. 27 is a diagram showing the results of a cold plate test in which D-threitol was administered to the mice that had developed peripheral neuropathy induced by paclitaxel.

FIG. 28 is a diagram showing the results of a von Frey test in which D-threitol was administered to the mice that had developed peripheral neuropathy induced by paclitaxel.

FIG. 29 is a diagram showing the results of a cold plate test in which D-threitol was administered to the mice that had developed peripheral neuropathy induced by vincristine.

FIG. 30 is a diagram showing the results of a von Frey test in which D-threitol was administered to the mice that had developed peripheral neuropathy induced by vincristine.

FIG. 31 is a diagram showing the results of a cold plate test in which D-threitol was administered to the mice that had developed peripheral neuropathy induced by bortezomib.

FIG. 32 is a diagram showing the results of a von Frey test in which D-threitol was administered to the mice that had developed peripheral neuropathy induced by bortezomib.

FIG. 33 is a diagram showing the results of a cold plate test in which L-talitol was administered to the mice together with oxaliplatin.

FIG. 34 is a diagram showing the results of a von Frey test in which L-talitol was administered to the mice together with oxaliplatin.

FIG. 35 is a diagram showing the results of a cold plate test in which L-talitol was administered to the mice together with paclitaxel.

FIG. 36 is a diagram showing the results of a von Frey test in which L-talitol was administered to the mice together with paclitaxel.

FIG. 37 is a diagram showing the results of a cold plate test in which L-talitol was administered to the mice together with vincristine.

FIG. 38 is a diagram showing the results of a von Frey test in which L-talitol was administered to the mice together with vincristine.

FIG. 39 is a diagram showing the results of a cold plate test in which L-talitol was administered to the mice together with bortezomib.

FIG. 40 is a diagram showing the results of a von Frey test in which L-talitol was administered to the mice together with bortezomib.

FIG. 41 is a diagram showing the results of a cold plate test in which L-talitol was administered to the mice that had developed peripheral neuropathy induced by oxaliplatin.

FIG. 42 is a diagram showing the results of a von Frey test in which L-talitol was administered to the mice that had developed peripheral neuropathy induced by oxaliplatin.

FIG. 43 is a diagram showing the results of a cold plate test in which L-talitol was administered to the mice that had developed peripheral neuropathy induced by paclitaxel.

FIG. 44 is a diagram showing the results of a von Frey test in which L-talitol was administered to the mice that had developed peripheral neuropathy induced by paclitaxel.

FIG. 45 is a diagram showing the results of a cold plate test in which L-talitol was administered to the mice that had developed peripheral neuropathy induced by vincristine.

FIG. 46 is a diagram showing the results of a von Frey test in which L-talitol was administered to the mice that had developed peripheral neuropathy induced by vincristine.

FIG. 47 is a diagram showing the results of a cold plate test in which L-talitol was administered to the mice that had developed peripheral neuropathy induced by bortezomib.

FIG. 48 is a diagram showing the results of a von Frey test in which L-talitol was administered to the mice that had developed peripheral neuropathy induced by bortezomib.

FIG. 49 is a diagram showing the results of a cold plate test in which xylitol administration was started on the first day of streptozotocin administration to the mice.

FIG. 50 is a diagram showing the results of a von Frey test in which xylitol administration was started on the first day of streptozotocin administration to the mice.

FIG. 51 is a diagram showing the results of a cold plate test in which xylitol was administered to the mice that had developed diabetes, which was induced by administration of streptozotocin, and developed peripheral neuropathy.

FIG. 52 is a diagram showing the results of a von Frey test in which xylitol was administered to the mice that had developed diabetes, which was induced by administration of streptozotocin, and developed peripheral neuropathy.

FIG. 53 is a diagram showing the results of a cold plate test in which D-threitol administration was started on the first day of streptozotocin administration to the mice.

FIG. 54 is a diagram showing the results of a von Frey test in which D-threitol administration was started on the first day of streptozotocin administration to the mice.

FIG. 55 is a diagram showing the results of a cold plate test in which D-threitol was administered to the mice that had developed diabetes, which was induced by administration of streptozotocin, and developed peripheral neuropathy.

FIG. 56 is a diagram showing the results of a von Frey test in which D-threitol was administered to the mice that had developed diabetes, which was induced by administration of streptozotocin, and developed peripheral neuropathy.

FIG. 57 is a diagram showing the results of a cold plate test in which L-talitol administration was started on the first day of streptozotocin administration to the mice.

FIG. 58 is a diagram showing the results of a von Frey test in which L-talitol administration was started on the first day of streptozotocin administration to the mice.

FIG. 59 is a diagram showing the results of a cold plate test in which L-talitol was administered to the mice that had developed diabetes, which was induced by administration of streptozotocin, and developed peripheral neuropathy.

FIG. 60 is a diagram showing the results of a von Frey test in which L-talitol was administered to the mice that had developed diabetes, which was induced by administration of streptozotocin, and developed peripheral neuropathy.

FIG. 61 is a diagram showing the result of a suppression effect of xylitol on nerve outgrowth inhibition of peripheral nerves using a cell line PC12 derived from rat pheochromocytoma.

FIG. 62 is a diagram showing the result of a cytotoxicity test of peripheral nerves with xylitol using the cell line PC12 derived from rat pheochromocytoma.

FIG. 63 is a diagram showing the result of the suppression effect of xylitol on the nerve outgrowth inhibition of the peripheral nerves using a human neuroblastoma cell line SH-SY5Y.

FIG. 64 is a diagram showing the result of the cytotoxicity test of peripheral nerves with xylitol using the human neuroblastoma cell line SH-SY5Y.

### DESCRIPTION OF EMBODIMENTS

A preventive or ameliorative agent according to the present invention will be described below with reference to drawings and examples. Note that the following description shows one embodiment and one example of the present invention, and the present invention is not limited to the following description. The following description can be modified without departing from the gist of the present invention.

In the context of the preventive or ameliorative agent according to the present invention, the term “prevention” refers to not only the prevention of the onset of peripheral neuropathy but also the action of reducing the degree of its symptoms at the onset; and the term “amelioration” refers to not only a root cause treatment of peripheral neuropathy but also the action of reducing or alleviating the degree of symptoms of peripheral neuropathy. As used herein, “to” written between two numerical values refers to a range of “the first value or more, and the second value or less.”

The preventive or ameliorative agent according to the present invention contains, as an active ingredient, at least one or more selected from xylitol (CAS No. 87-99-0), L-talitol (CAS No. 60660-58-4), and D-threitol (CAS No. 2418-52-2). Xylitol, L-talitol, and D-threitol (hereinafter, referred to as “xylitol or the like” when collectively described) used in the preventive or ameliorative agent according to the present invention can be prepared by a method conventionally known in the technical field of interest.

For example, xylitol is mass-produced by a method in which xylan is extracted from sources such as corn stalks and/or silver birch, and hydrolyzed to be refined into xylose, and the xylose is hydrogenated using nickel as a catalyst. L-talitol can be produced by an organic chemical method in which a monosaccharide such as L-talose or L-altrose is reduced with hydrogen under high temperature and high pressure in the presence of a metal catalyst. A method of obtaining the same by enzymatic reaction is also disclosed. Generally, D-threitol is produced via modification of the corresponding isomer of tartaric acid.

The preventive or ameliorative agent according to the present invention can be provided in the form of a pharmaceutical product, a food product, or the like. The ameliorative agent according to the present invention can also be provided with a label indicating that it is for ameliorating peripheral neuropathy and/or preventing peripheral neuropathy.

When used as a pharmaceutical product, the preventive or ameliorative agent according to the present invention can be provided as a therapeutic agent for peripheral neuropathy (pharmaceutical composition) or a preventive agent for peripheral neuropathy (pharmaceutical composition).

When used as a pharmaceutical product, the preventive or ameliorative agent according to the present invention may be administered by any of the methods including oral, transdermal, enteral, intravenous, pulmonary, subcutaneous, transmucosal, or intramuscular administration, and the method may be appropriately specified depending on, for example, the degree of peripheral neuropathy to be prevented or ameliorated.

When the preventive or ameliorative agent according to the present invention is used as a pharmaceutical product, xylitol or the like may be formulated, alone or in combination with any other substance such as an additive, into a desired dosage form. Specific examples of the pharmaceutical product include an oral preparation such as a capsule, a granule, a powder, a pill, a tablet, a jelly, and a syrup; an external preparation such as a liquid, an ointment, a cream, a lotion, a gel a patch, and an aerosol; and an injectable preparation.

When the preventive or ameliorative agent according to the present invention is used as a pharmaceutical product, an additive such as a binder, a lubricant, a disintegrating agent, a coloring agent, a flavoring agent, an antiseptic agent, an antioxidant, a stabilizing agent, water, a lower alcohol, a solubilizing agent, a surfactant, an emulsion stabilizer, a gelling agent, an adhesive, a flavor, or a coloring matter may be selected as appropriate to produce a preparation in a desired dosage form. Furthermore, the pharmaceutical product may contain a pharmacological ingredient such as a vasodilator, an adrenocortical hormone, a keratolytic agent, a moisturizing agent, a microbicide, an antioxidant, or a cooling agent, as needed.

When the preventive or ameliorative agent according to the present invention is used as a pharmaceutical product, the content of xylitol or the like in the pharmaceutical product may be appropriately set depending on, for example, the dosage form of the pharmaceutical product so as to satisfy a daily dose thereof described below. For example, in the case of an oral preparation, the total amount of xylitol or the like is, for example, 0.1 to 100% by mass, preferably 15 to 80% by mass, and more preferably 30 to 70% by mass; in the case of an external preparation, the total amount of xylitol or the like is, for example, 0.01 to 50% by mass, preferably 0.1 to 40% by mass, and more preferably 0.5 to 30% by mass.

When the preventive or ameliorative agent according to the present invention is used as a food product, the food product is provided as a food product for preventing or ameliorating peripheral neuropathy.

When the preventive or ameliorative agent according to the present invention is used as a food product, xylitol or the like may be prepared, alone or in combination with any other food material and/or additive ingredient, into a desired form. Examples of the food product include a general processed food such as a food for pleasure or a health food; and a food with health claims such as a food for specified health uses, a food with nutrient function claims, or a functional food defined in the Food with Health Claims System by the Ministry of Health, Labor and Welfare. Specific examples of the food product include a general processed food, such as a food for pleasure such as a candy, gum, a gelatin dessert, a biscuit, a cookie, a rice cracker, bread, yogurt, ice cream, and a custard pudding; noodles; a food product made from fish paste and/or meat paste; a beverage such as tea, a refreshing drink, a coffee beverage, a milk beverage, a whey beverage, and a lactic acid bacteria beverage; and a supplement such as a capsule (a soft capsule and a hard capsule), a tablet, a granule, a powdered medicine, and a jelly. Among these foods and beverages, a supplement is preferred.

When the preventive or ameliorative agent according to the present invention is used as a food product, the content of xylitol or the like in the food product may be appropriately set depending on, for example, the type of the food product so as to satisfy a daily intake thereof described below. For example, the total amount of xylitol or the like is 0.05 to 100% by mass, preferably 10 to 80% by mass, and more preferably 20 to 60% by mass.

The preventive or ameliorative agent according to the present invention is used for preventing or ameliorating peripheral neuropathy. Examples of the symptom of peripheral neuropathy to which the preventive or ameliorative agent according to the present invention is applied include sensory neuropathy, autonomic neuropathy, and motor neuropathy. The symptom of peripheral neuropathy to which the preventive or ameliorative agent according to the present invention is applied is more preferably sensory neuropathy. Examples of the sensory neuropathy include, but are not particularly limited to, numbness in the limbs, pain in the limbs, decreased deep tendon reflexes, muscle weakness, allodynia, hyperalgesia, paralgesia, impairment of hand dexterity, gait disorder, stumble, a fall, a flexion disorder (difficulty or incapability of sitting straight, sitting cross-legged, sitting with one's legs bent back to one side, sitting on a chair, or the like), and limb paralysis.

Prevention or amelioration of peripheral neuropathy can provide, but not particularly limited to, assistance of peripheral neurotransmission of limbs (including hands and feet), assistance of hand movement in daily life such as writing characters and/or letters or doing up a button, assistance of grip strength or a sense of putting strength in a hand, and a reduction in temporary discomfort or uncomfortable feeling in the hand. In short, it may be said that the preventive or ameliorative agent according to the present invention reduces discomfort in the hands and feet or assists neurotransmission in the hands and feet.

Furthermore, there is no particular limitation to a trigger for peripheral neuropathy to which the preventive or ameliorative agent according to the present invention is applied. For example, the preventive or ameliorative agent according to the present invention can be applied to any peripheral neuropathy that is caused by cancer chemotherapy, administration of other drugs, progression of diabetes, trauma, infectious disease, and the like. In particular, the agent is suitably applied to peripheral neuropathy induced by cancer chemotherapy or diabetic peripheral neuropathy.

When the preventive or ameliorative agent according to the present invention is applied to peripheral neuropathy that is induced by various anticancer drugs, the type of anticancer drug is not particularly limited. Examples of the anticancer drug include a platinum-containing drug, an alkylating agent, an antimetabolite, a microtubule-affecting drug, an anticancer antibiotic, a topoisomerase inhibitor, a proteasome inhibitor, a histone deacetylase inhibitor, a FLT3 tyrosine kinase inhibitor, an antibody drug, an ALK inhibitor, a HER2/EGFR tyrosine kinase inhibitor, an ALK/ROS1 tyrosine kinase inhibitor, a TRK/ROS1 tyrosine kinase inhibitor, a multi-kinase inhibitor, a JAK inhibitor, a BCR-ABL inhibitor, an FGFR inhibitor, a MET inhibitor, a BRAF inhibitor, a MEK inhibitor, an immunomodulator, and an immune checkpoint inhibitor.

Specific examples of the platinum-containing drug include oxaliplatin, cisplatin, carboplatin, and nedaplatin. Specific examples of the alkylating agent include cyclophosphamide, ifosfamide, melphalan, thiotepa, carboquone, nimustine hydrochloride, ranimustine, carmustine, and busulfan.

Specific examples of the antimetabolite include 5-fluorouracil, methotrexate, doxifluridine, tegafur, cytarabine, cytarabine ocfosphate, enocitabine, gemcitabine, mercaptopurine, fludarabine, capecitabine, and azacytidine. Specific examples of the microtubule-affecting drug include docetaxel, paclitaxel, vincristine, vinblastine, vindesine, vinorelbine, cabazitaxel, and eribulin.

Specific examples of the anticancer antibiotic include doxorubicin hydrochloride, mitomycin, amrubicin hydrochloride, pirarubicin hydrochloride, epirubicin hydrochloride, aclarubicin hydrochloride, mitoxantrone hydrochloride, bleomycin hydrochloride, peplomycin sulfate, daunorubicin, idarubicin, and actinomycin D. Specific examples of the topoisomerase inhibitor include irinotecan, nogitecan hydrochloride, and etoposide.

Specific examples of the proteasome inhibitor include bortezomib, carfilzomib, and ixazomib. Specific examples of the histone deacetylase inhibitor include vorinostat, panobinostat, romidepsin, and tucidinostat.

Specific examples of the FLT3 tyrosine kinase inhibitor include gilteritinib. Specific examples of the antibody drug include pertuzumab, trastuzumab emtansine, brentuximab vedotin, polatuzumab vedotin, rituximab, obinutuzumab, blinatumomab, bevacizumab, mogamulizumab, ofatumumab, ibritumomab tiuxetan, gemtuzumab ozogamicin, inotuzumab ozogamicin, alemtuzumab, daratumumab, isatuximab, elotuzumab, trastuzumab, tastuzumab deruxtecan, cetuximab, panitumumab, necitumumab, cetuximab sarotalocan sodium, ramucirumab, dinutuximab, aflibercept beta, and denosumab.

Specific examples of the ALK inhibitor include alectinib, brigtinib, and ceritinib. Specific examples of the HER2/EGFR tyrosine kinase inhibitor include lapatinib. Specific examples of the ALK/ROS1 tyrosine kinase inhibitor include crizotinib and lorlatinib.

Specific examples of the TRK/ROS1 tyrosine kinase inhibitor include larotrectinib and entrectinib. Specific examples of the multi-kinase inhibitor include sorafenib, sunitinib, pazopanib, vandetanib, axitinib, regorafenib, nintedanib, lenvatinib, and cabozantinib.

Specific examples of the JAK inhibitor include ruxolitinib. Specific examples of the BCR-ABL inhibitor include imatinib, nilotinib, dasatinib, bosutinib, and ponatinib. Specific examples of the FGFR inhibitor include pemigatinib. Specific examples of the MET inhibitor include tepotinib and capmatinib.

Specific examples of the BRAF inhibitor include vemurafenib, dabrafenib, and encorafenib. Specific examples of the MEK inhibitor include binimetinib and trametinib. Specific examples of the immunomodulator include thalidomide, lenalidomide, and pomalidomide. Specific examples of the immune checkpoint inhibitor include nivolumab, ipilimumab, pembrolizumab, atezolizumab, avelumab, and durvalumab.

When the preventive or ameliorative agent according to the present invention is applied to peripheral neuropathy that is induced by cancer chemotherapy, there is no particular limitation to the type of an anticancer drug that triggers the peripheral neuropathy of interest. Preferred examples of the anticancer drug include a DNA replication inhibitor (a platinum-containing agent and an alkylating agent), a microtubule polymerization stabilizer, a microtubule polymerization inhibitor, and a proteasome inhibitor.

When the preventive or ameliorative agent according to the present invention is applied to peripheral neuropathy that is induced by cancer chemotherapy, the administration or intake of the preventive or ameliorative agent according to the present invention may be started before or at the same time as the start of administration of the cancer chemotherapy. However, the administration or intake of the preventive or ameliorative agent according to the present invention may also be started during the cancer chemotherapy or after the end the cancer chemotherapy.

When the preventive or ameliorative agent according to the present invention is applied to diabetic peripheral neuropathy, the agent can be taken as an ameliorative agent for the peripheral neuropathy after the onset thereof. Furthermore, even before the peripheral neuropathy is developed, if the onset of diabetes can be confirmed, the agent can be taken and used as a preventive agent.

The dose or intake of the preventive or ameliorative agent according to the present invention can be appropriately selected depending on the symptom, age, body weight, time since onset, and concurrent therapeutic measures. In the examples of the present invention, when an anticancer drug was administered in an amount sufficient to cause peripheral neuropathy (for example, 6 mg oxaliplatin/kg mouse body weight), the total daily intake of xylitol or the like per mouse that is effective for ameliorating the peripheral neuropathy is as follows. The total daily intake may be 5 mg/kg mouse body weight or more for prevention, and is preferably 100 mg/kg mouse body weight or more for treatment. In the examples of the present invention, when streptozotocin was administered at a dose of 200 mg/kg mouse weight to induce diabetes and thus peripheral neuropathy, the total daily intake of xylitol or the like per mouse that is effective for ameliorating the peripheral neuropathy is as follows. The total daily intake may be 1 mg/kg mouse body weight or more, and is preferably 5 mg/kg mouse body weight or more.

In the technical field of interest, when a certain ingredient is considered to be effective in mice, a dose thereof that produces an equivalent effect in humans is calculated as a human equivalent dose (HED). When the weight of a mouse is defined as 30 g and the weight of a human is defined as 60 kg, the value obtained by dividing the dose for a mouse by 12.3 is considered to be the human equivalent dose.

Based on the HED, the total daily intake of xylitol or the like for ameliorating the peripheral neuropathy that is caused by an anticancer drug may be 0.41 mg/kg human body weight or more for prevention, and is preferably 8.13 mg/kg human body weight or more for treatment. Therefore, the total daily intake of xylitol or the like taken daily by an adult human male may be 24.6 mg/day/adult or more for prevention, and is preferably 487.8 mg/day/adult or more for treatment. The total daily intake of xylitol or the like for ameliorating the diabetic peripheral neuropathy may be 0.08 mg/kg human body weight or more for prevention, and is preferably 0.41 mg/kg human body weight or more for treatment. Therefore, the total daily intake of xylitol or the like taken daily by an adult human male may be 4.8 mg/day/adult or more for prevention, and is preferably 24.6 mg/day/adult or more for treatment.

In this regard, excessive intake of xylitol or the like is believed to cause diarrhea and its limit dose is 20 to 30 g/day, although there are differences between individuals. Therefore, it is believed that, in many cases, an amount necessary for ameliorating peripheral neuropathy hardly causes side effects. Therefore, when the preventive or ameliorative agent according to the present invention is orally administered or taken, the total dose or intake of xylitol or the like may be 0.25 g/day/adult to 30 g/day/adult, and is preferably 0.48 g/day/adult to 20 g/day/adult or less. In the case of diabetes, a smaller dose (4.8 mg/day/adult to 30 g/day/adult, preferably 24.6 mg/day/adult to 20 g/day/adult) can be used.

The preventive or ameliorative agent according to the present invention may be administered or taken once per day or 2 to 3 times per day in such a manner as to satisfy the daily dose or intake thereof.

### EXAMPLES

### (Example 1) Xylitol-Containing Preventive Agent for Peripheral Neuropathy that is Caused by Administration of Anticancer Drug

### <Preventive Action of Xylitol on Peripheral Neuropathy in a Mouse Caused by Oxaliplatin>

The preventive action of xylitol on hyperesthesia that occurs when an anticancer drug, oxaliplatin, was administered was examined. Examples of hyperesthesia include paresthesia that is caused by a low temperature stimulus and allodynia (severe pain induced by a tactile stimulus that does not usually cause pain) that is caused by a mechanical stimulus. Xylitol was orally administered to a mouse simultaneously with the administration of oxaliplatin, and the following tests (cold plate test and von Frey test) were performed. Oxaliplatin is an anticancer drug classified as a platinum-containing drug. In all of the examples described below, a dosage (administered amount) (mg/kg) refers to the weight of the administered substance per kg body weight of the mouse.

(1) Administration of Test Article

Six to seven-week-old female Balb/c mice were used for the tests. All the mice were acclimated for seven days after arrival. Then, the mice were grouped into the following four groups: a control group, a group receiving oxaliplatin, and two groups receiving oxaliplatin and two different doses of xylitol (groups receiving oxaliplatin+xylitol). Each group consisted of nine mice.

On Day 8 of acclimation, the mice in the group receiving oxaliplatin and the groups receiving oxaliplatin+xylitol were intraperitoneally administered with oxaliplatin at a dose of 6 mg/kg. This day was designated as Day 0 of administration. Thereafter, these mice were intraperitoneally administered with oxaliplatin at a daily dose of 6 mg/kg on Day 7 and Day 14.

The mice in a first group receiving oxaliplatin and xylitol were orally administered with xylitol at a dose of 1 mg/kg daily from Day 0. The mice in a second group receiving oxaliplatin and xylitol were orally administered with xylitol at a dose of 5 mg/kg daily from Day 0.

These respective groups are referred to as the control group, the group receiving 6 mg/kg oxaliplatin, the group receiving 6 mg/kg oxaliplatin+1 mg/kg xylitol, and the group receiving 6 mg/kg oxaliplatin+5 mg/kg xylitol.

(2) Cold Plate Test

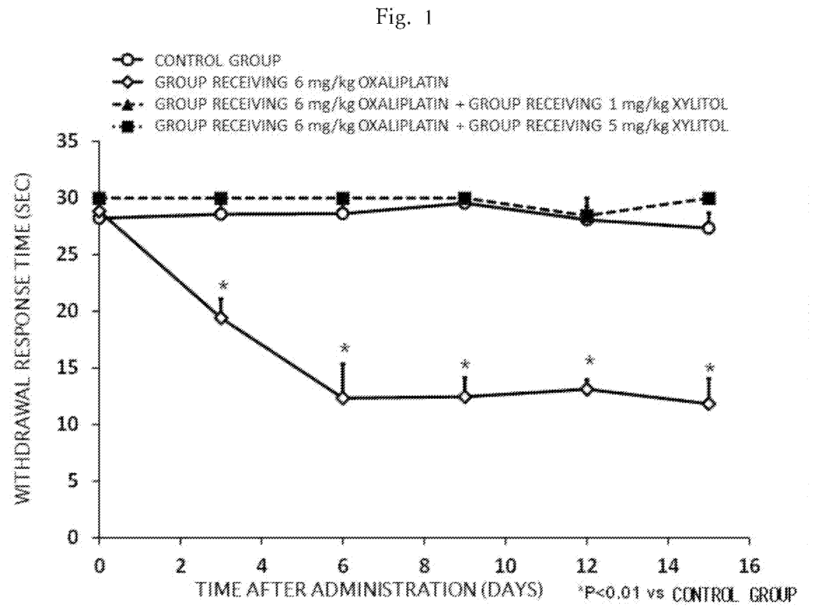

A cold plate test was performed to examine the effect of xylitol on paresthesia that is caused by a low temperature stimulus. The mice in the four groups described in (1) of the present example (Example 1) were placed on a cold plate set at 10° C., and withdrawal response time (latent time) was measured. It is presumed that a shorter latent time reflects a greater degree of shunning the low temperature stimulus by the cold plate. The results are shown in FIG. 1.

With reference to FIG. 1, the horizontal axis represents the time after administration (days), and the vertical axis represents the average value of the withdrawal response time (seconds) of the mice in each group. The solid line with white circles represents the control group; the solid line with white diamonds represents the group receiving 6 mg/kg oxaliplatin; the dotted line with black triangles represents the group receiving 6 mg/kg oxaliplatin+1 mg/kg xylitol; and the broken line with black squares represents the group receiving 6 mg/kg oxaliplatin+5 mg/kg xylitol. A “*” mark was added to a measurement that was determined to have a significant difference from that of the control group in a test, using a significance level of 1% (in the figure, shown as “*P<0.01 vs control group”).

In the group receiving oxaliplatin (solid line with white diamonds), the withdrawal response time (latent time) when given a cold stimulus by the cold plate was reduced from Day 3 to Day 6 of the test, and thereafter, the withdrawal response time remained constant. On the other hand, the group receiving 6 mg/kg oxaliplatin+1 mg/kg xylitol (dotted line with black triangles) and the group receiving 6 mg/kg oxaliplatin+5 mg/kg xylitol (broken line with black squares), which received xylitol in combination, exhibited a withdrawal response time (latent time) approximately similar to that of the control group (solid line with white circles). The two groups receiving xylitol also did not show a reduction in the withdrawal response time thereafter. In these two groups, the reduction in the withdrawal response time (latent time) was suppressed compared with the group receiving 6 mg/kg oxaliplatin (solid line with white diamonds).

(3) Von Frey Test

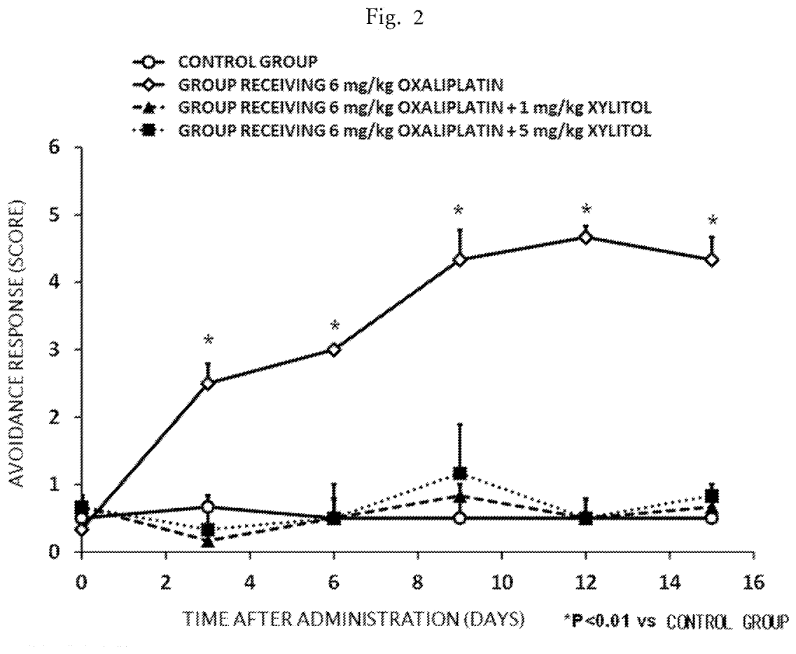

The mice in the four groups described in (1) of the present example (Example 1) were placed in a cage, and the avoidance response frequency (score) was measured by pressing a filament with a bending force of 0.16 g against the back of the hind paw. The results are shown in FIG. 2.

With reference to FIG. 2, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the solid line with white diamonds represents the group receiving 6 mg/kg oxaliplatin; the dotted line with black triangles represents the group receiving 6 mg/kg oxaliplatin+1 mg/kg xylitol; and the broken line with black squares represents the group receiving 6 mg/kg oxaliplatin+5 mg/kg xylitol. A “*” mark was added to a measurement that was determined to have a significant difference from that of the control group in a test, using a significance level of 1% (in the figure, shown as “*P<0.01 vs control group”).

The group receiving oxaliplatin (solid line with white diamonds) exhibited a remarkable increase in the avoidance response score compared with the control group (solid line with white circles). On the other hand, the group receiving 6 mg/kg oxaliplatin+1 mg/kg xylitol (dotted line with black triangles) and the group receiving 6 mg/kg oxaliplatin+5 mg/kg xylitol (broken line with black squares), which received xylitol in combination, exhibited avoidance response scores similar to that of the control group throughout the experimental period. In these two groups, an increase in the avoidance response score was suppressed compared with the group receiving oxaliplatin (solid line with white diamonds).

Both the cold plate test (FIG. 1) and the von Frey test (FIG. 2) demonstrated that the group receiving oxaliplatin (solid line with white diamonds) exhibited a significant change in both the avoidance response score and the withdrawal response time compared with the control group (solid line with white circles), the change being a shift to a greater degree of shunning the stimulus. This demonstrates that the administration of oxaliplatin caused peripheral neuropathy (peripheral nerve hypersensitivity) to develop.

On the other hand, the group receiving 6 mg/kg oxaliplatin+1 mg/kg xylitol (dotted line with black triangles) and the group receiving 6 mg/kg oxaliplatin+5 mg/kg xylitol (broken line with black squares), which received xylitol, showed results similar to those of the control group. Therefore, it can be concluded that xylitol suppresses the peripheral neuropathy (peripheral nerve hypersensitivity) induced by oxaliplatin. In other words, xylitol serves as a preventive composition (preventive agent) for peripheral neuropathy that is caused by oxaliplatin (anticancer drug).

<Preventive Action of Xylitol on Peripheral Neuropathy in a Mouse Caused by Paclitaxel>

The preventive action of xylitol on hyperesthesia that occurs when an anticancer drug, paclitaxel, was administered was examined. Examples of hyperesthesia include paresthesia that is caused by a low temperature stimulus and allodynia (severe pain induced by a tactile stimulus that does not usually cause pain) that is caused by a mechanical stimulus. Xylitol was orally administered to a mouse simultaneously with the administration of paclitaxel, and the following tests (cold plate test and von Frey test) were performed. Paclitaxel is an anticancer drug classified as a microtubule polymerization stabilizer (microtubule-affecting drug).

(4) Administration of Test Article

Six to seven-week-old female Balb/c mice were used for the tests. All the mice were acclimated for seven days after arrival. Then, the mice were grouped into the following four groups: a control group, a group receiving paclitaxel, a group receiving xylitol, and a group receiving paclitaxel and xylitol (a group receiving paclitaxel+xylitol). Each group consisted of five mice.

On Day 8 of acclimation, the mice in the group receiving paclitaxel and the group receiving paclitaxel+xylitol were intraperitoneally administered with paclitaxel at a dose of 6 mg/kg. This day was designated as Day 0 of administration. Thereafter, these mice were intraperitoneally administered with paclitaxel at a daily dose of 6 mg/kg on Day 7 and Day 14.

The mice in the group receiving xylitol and the group receiving paclitaxel+xylitol were orally administered with xylitol at a dose of 5 mg/kg daily from Day 0.

These respective groups are referred to as the control group, the group receiving 6 mg/kg paclitaxel, the group receiving 5 mg/kg xylitol, and the group receiving 6 mg/kg paclitaxel+5 mg/kg xylitol.

(5) Cold Plate Test

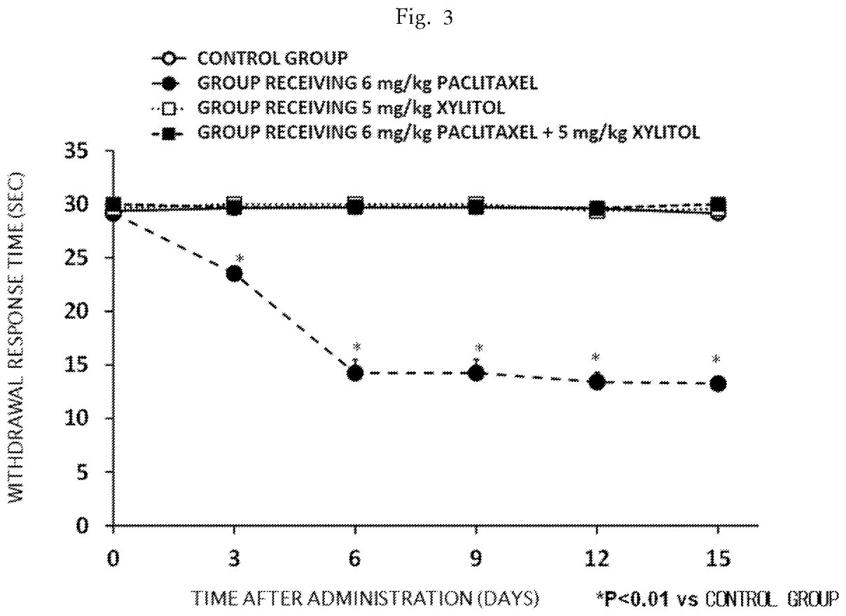

A cold plate test was performed to examine the effect of xylitol on paresthesia that is caused by a low temperature stimulus. The mice in the four groups described in (4) of the present example (Example 1) were placed on a cold plate set at 10° C., and withdrawal response time (latent time) was measured. It is presumed that a shorter latent time reflects a greater degree of shunning the low temperature stimulus by the cold plate. The results are shown in FIG. 3.

With reference to FIG. 3, the horizontal axis represents the time after administration (days), and the vertical axis represents the average value of the withdrawal response time (seconds) of the mice in each group. The solid line with white circles represents the control group; the broken line with black circles represents the group receiving 6 mg/kg paclitaxel; the dotted line with white squares represents the group receiving 5 mg/kg xylitol; and the broken line with black squares represents the group receiving 6 mg/kg paclitaxel+5 mg/kg xylitol. A “*” mark was added to a measurement that was determined to have a significant difference from that of the control group in a test, using a significance level of 1% (in the figure, shown as “*P<0.01 vs control group”).

In the group receiving 6 mg/kg paclitaxel (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg xylitol (dotted line with white squares), which received only xylitol, and the group receiving 6 mg/kg paclitaxel+5 mg/kg xylitol (broken line with black squares), which received paclitaxel and xylitol in combination, exhibited a withdrawal response time (latent time) approximately similar to that of the control group (solid line with white circles). The two groups receiving xylitol also did not show a reduction in the withdrawal response time thereafter. In these two groups, the reduction in the withdrawal response time (latent time) was suppressed compared with the group receiving 6 mg/kg paclitaxel (broken line with black circles).

(6) Von Frey Test

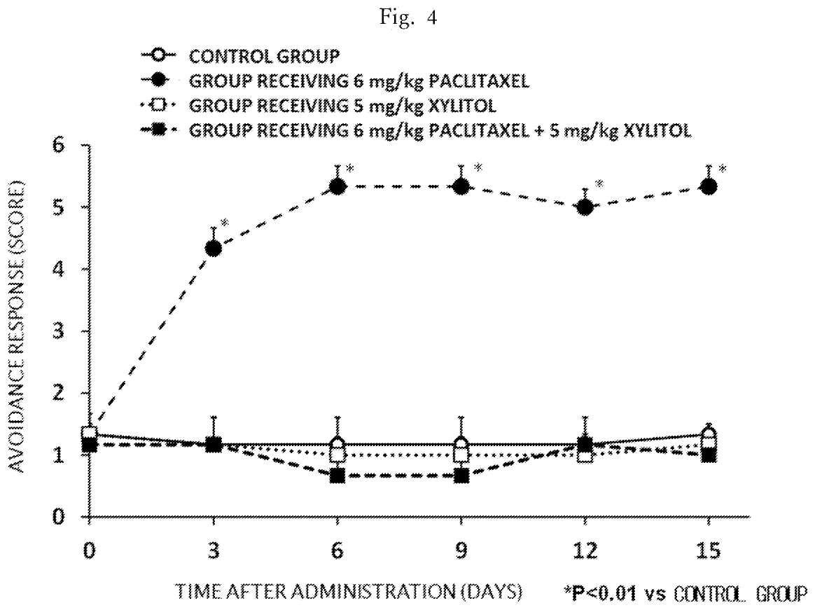

The mice in the four groups described in (4) of the present example (Example 1) were placed in a cage, and the avoidance response frequency (score) was measured by pressing a filament with a bending force of 0.16 g against the back of the hind paw. The results are shown in FIG. 4.

With reference to FIG. 4, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 6 mg/kg paclitaxel; the dotted line with white squares represents the group receiving 5 mg/kg xylitol; and the broken line with black squares represents the group receiving 6 mg/kg paclitaxel+5 mg/kg xylitol. A “*” mark was added to a measurement that was determined to have a significant difference from that of the control group in a test, using a significance level of 1% (in the figure, shown as “*P<0.01 vs control group”).

The group receiving 6 mg/kg paclitaxel (broken line with black circles) exhibited a remarkable increase in the avoidance response score compared with the control group (solid line with white circles). On the other hand, the group receiving 5 mg/kg xylitol (dotted line with white squares), which received only xylitol, and the group receiving 6 mg/kg paclitaxel+5 mg/kg xylitol (broken line with black squares), which received xylitol in combination, exhibited avoidance response scores similar to that of the control group (solid line with white circles) throughout the experimental period. In these two groups, an increase in the avoidance response score was suppressed compared with the group receiving 6 mg/kg paclitaxel (broken line with black circles).

Both the cold plate test (FIG. 3) and the von Frey test (FIG. 4) demonstrated that the group receiving 6 mg/kg paclitaxel (broken line with black circles) exhibited a significant change in both the withdrawal response time and the avoidance response score compared with the control group (solid line with white circles), the change being a shift to a greater degree of shunning the stimulus. This demonstrates that the administration of paclitaxel caused peripheral neuropathy (peripheral nerve hypersensitivity) to develop.

On the other hand, the group receiving 5 mg/kg xylitol (dotted line with white squares) and the group receiving 6 mg/kg paclitaxel+5 mg/kg xylitol (broken line with black squares), which received xylitol, showed results similar to those of the control group. Therefore, it can be concluded that xylitol suppresses the peripheral neuropathy (peripheral nerve hypersensitivity) induced by paclitaxel. In other words, xylitol serves as a preventive composition (preventive agent) for peripheral neuropathy that is caused by paclitaxel (anticancer drug).

<Preventive Action of Xylitol on Peripheral Neuropathy in a Mouse Caused by Vincristine>

The preventive action of xylitol on hyperesthesia that occurs when an anticancer drug, vincristine, was administered was examined. Examples of hyperesthesia include paresthesia that is caused by a low temperature stimulus and allodynia (severe pain induced by a tactile stimulus that does not usually cause pain) that is caused by a mechanical stimulus. Xylitol was orally administered to a mouse simultaneously with the administration of vincristine, and the following tests (cold plate test and von Frey test) were performed. Vincristine is an anticancer drug classified as a microtubule polymerization inhibitor (microtubule-affecting drug).

(7) Administration of Test Article

Six to seven-week-old female Balb/c mice were used for the tests. All the mice were acclimated for seven days after arrival. Then, the mice were grouped into the following four groups: a control group, a group receiving vincristine, a group receiving xylitol, and a group receiving vincristine and xylitol (a group receiving vincristine+xylitol). Each group consisted of five mice.

On Day 8 of acclimation, the mice in the group receiving vincristine and the group receiving vincristine+xylitol were intraperitoneally administered with vincristine at a dose of 0.2 mg/kg. This day was designated as Day 0 of administration. Thereafter, these mice were intraperitoneally administered with vincristine at a daily dose of 0.2 mg/kg on Day 7 and Day 14.

The mice in the group receiving xylitol and the group receiving vincristine+xylitol were orally administered with xylitol at a dose of 5 mg/kg daily from Day 0.

These respective groups are referred to as the control group, the group receiving 0.2 mg/kg vincristine, the group receiving 5 mg/kg xylitol, and the group receiving 0.2 mg/kg vincristine+5 mg/kg xylitol.

(8) Cold Plate Test

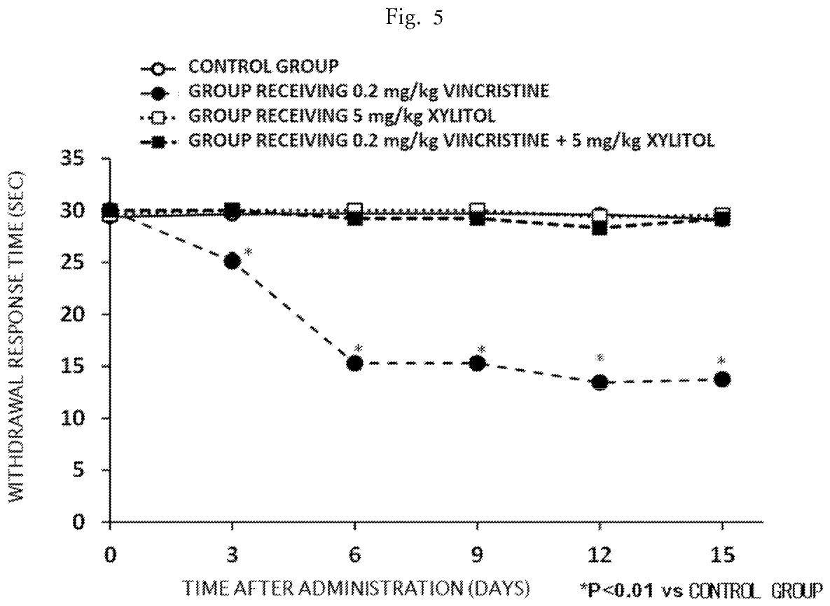

A cold plate test was performed to examine the effect of xylitol on paresthesia that is caused by a low temperature stimulus. The mice in the four groups described in (7) of the present example (Example 1) were placed on a cold plate set at 10° C., and withdrawal response time (latent time) was measured. It is presumed that a shorter latent time reflects a greater degree of shunning the low temperature stimulus by the cold plate. The results are shown in FIG. 5.

With reference to FIG. 5, the horizontal axis represents the time after administration (days), and the vertical axis represents the average value of the withdrawal response time (seconds) of the mice in each group. The solid line with white circles represents the control group; the broken line with black circles represents the group receiving 0.2 mg/kg vincristine; the dotted line with white squares represents the group receiving 5 mg/kg xylitol; and the broken line with black squares represents the group receiving 0.2 mg/kg vincristine+5 mg/kg xylitol. A “*” mark was added to a measurement that was determined to have a significant difference from that of the control group in a test, using a significance level of 1% (in the figure, shown as “*P<0.01 vs control group”).

In the group receiving 0.2 mg/kg vincristine (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg xylitol (dotted line with white squares), which received only xylitol, and the group receiving 0.2 mg/kg vincristine+5 mg/kg xylitol (broken line with black squares), which received vincristine and xylitol in combination, exhibited a withdrawal response time (latent time) approximately similar to that of the control group (solid line with white circles). The two groups receiving xylitol also did not show a reduction in the withdrawal response time thereafter. In these two groups, the reduction in the withdrawal response time (latent time) was suppressed compared with the group receiving 0.2 mg/kg vincristine (broken line with black circles).

(9) Von Frey Test

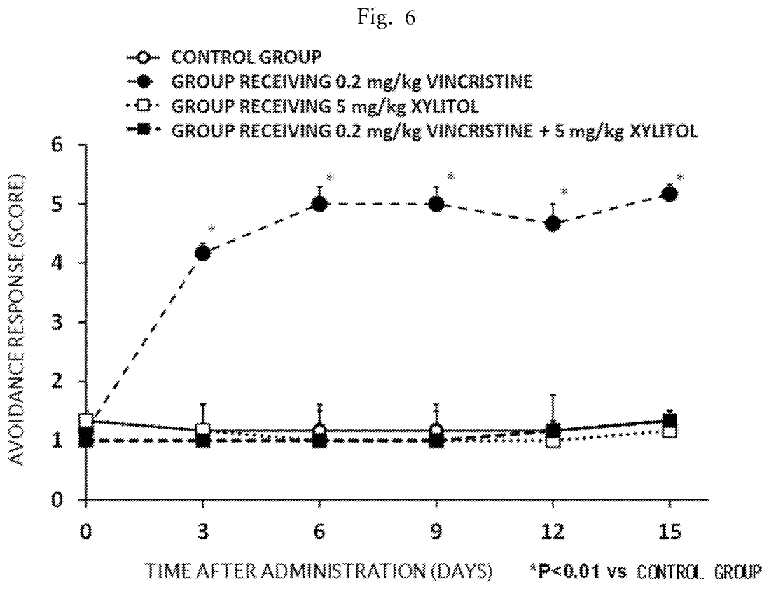

The mice in the four groups described in (7) of the present example (Example 1) were placed in a cage, and the avoidance response frequency (score) was measured by pressing a filament with a bending force of 0.16 g against the back of the hind paw. The results are shown in FIG. 6.

With reference to FIG. 6, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 0.2 mg/kg vincristine; the dotted line with white squares represents the group receiving 5 mg/kg xylitol; and the broken line with black squares represents the group receiving 0.2 mg/kg vincristine+5 mg/kg xylitol. A “*” mark was added to a measurement that was determined to have a significant difference from that of the control group in a test, using a significance level of 1% (in the figure, shown as “*P<0.01 vs control group”).

The group receiving 0.2 mg/kg vincristine (broken line with black circles) exhibited a remarkable increase in the avoidance response score compared with the control group (solid line with white circles). On the other hand, the group receiving 5 mg/kg xylitol (dotted line with white squares), which received only xylitol, and the group receiving 0.2 mg/kg vincristine+5 mg/kg xylitol (broken line with black squares), which received xylitol in combination, exhibited avoidance response scores similar to that of the control group (solid line with white circles) throughout the experimental period. In these two groups, an increase in the avoidance response score was suppressed compared with the group receiving 0.2 mg/kg vincristine (broken line with black circles).

Both the cold plate test (FIG. 5) and the von Frey test (FIG. 6) demonstrated that the group receiving 0.2 mg/kg vincristine (broken line with black circles) exhibited a significant change in both the withdrawal response time and the avoidance response score compared with the control group (solid line with white circles), the change being a shift to a greater degree of shunning the stimulus. This demonstrates that the administration of vincristine caused peripheral neuropathy (peripheral nerve hypersensitivity) to develop.

On the other hand, the group receiving 5 mg/kg xylitol (dotted line with white squares) and the group receiving 0.2 mg/kg vincristine+5 mg/kg xylitol (broken line with black squares), which received xylitol, showed results similar to those of the control group. Therefore, it can be concluded that xylitol suppresses the peripheral neuropathy (peripheral nerve hypersensitivity) induced by vincristine. In other words, xylitol serves as a preventive composition (preventive agent) for peripheral neuropathy that is caused by vincristine (anticancer drug).

<Preventive Action of Xylitol on Peripheral Neuropathy in a Mouse Caused by Bortezomib>

The preventive action of xylitol on hyperesthesia that occurs when an anticancer drug, bortezomib, was administered was examined. Examples of hyperesthesia include paresthesia that is caused by a low temperature stimulus and allodynia (severe pain induced by a tactile stimulus that does not usually cause pain) that is caused by a mechanical stimulus. Xylitol was orally administered to a mouse simultaneously with the administration of bortezomib, and the following tests (cold plate test and von Frey test) were performed. Bortezomib is an anticancer drug classified as a proteasome agent.

(10) Administration of Test Article

Six to seven-week-old female Balb/c mice were used for the tests. All the mice were acclimated for seven days after arrival. Then, the mice were grouped into the following four groups: a control group, a group receiving bortezomib, a group receiving xylitol, and a group receiving bortezomib and xylitol (a group receiving bortezomib+xylitol). Each group consisted of five mice.

On Day 8 of acclimation, the mice in the group receiving bortezomib and the group receiving bortezomib+xylitol were intraperitoneally administered with bortezomib at a dose of 1 mg/kg. This day was designated as Day 0 of administration. Thereafter, these mice were intraperitoneally administered with bortezomib at a daily dose of 1 mg/kg on Day 7 and Day 14.

The mice in the group receiving xylitol and the group receiving bortezomib+xylitol were orally administered with xylitol at a dose of 5 mg/kg daily from Day 0.

These respective groups are referred to as the control group, the group receiving 1 mg/kg bortezomib, the group receiving 5 mg/kg xylitol, and the group receiving 1 mg/kg bortezomib+5 mg/kg xylitol.

(11) Cold Plate Test

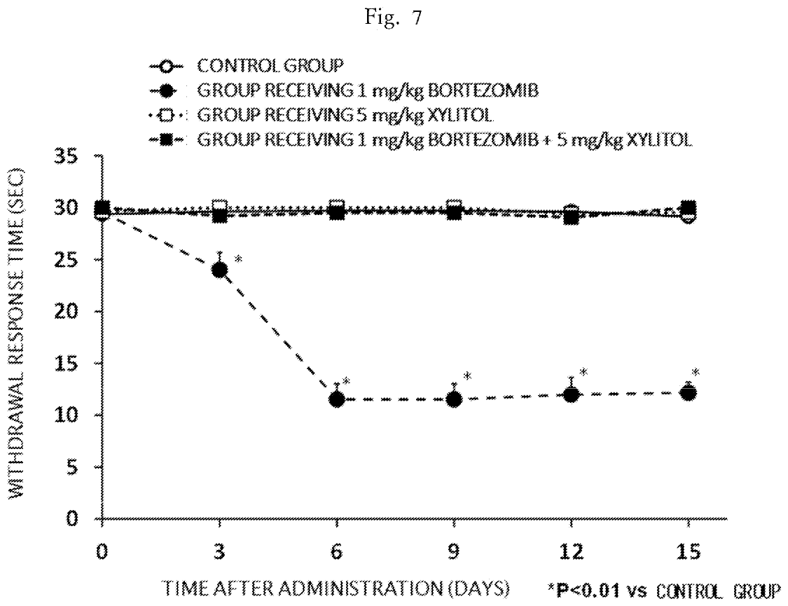

A cold plate test was performed to examine the effect of xylitol on paresthesia that is caused by a low temperature stimulus. The mice in the four groups described in (10) of the present example (Example 1) were placed on a cold plate set at 10° C., and withdrawal response time (latent time) was measured. It is presumed that a shorter latent time reflects a greater degree of shunning the low temperature stimulus by the cold plate. The results are shown in FIG. 7.

With reference to FIG. 7, the horizontal axis represents the time after administration (days), and the vertical axis represents the average value of the withdrawal response time (seconds) of the mice in each group. The solid line with white circles represents the control group; the broken line with black circles represents the group receiving 1 mg/kg bortezomib; the dotted line with white squares represents the group receiving 5 mg/kg xylitol; and the broken line with black squares represents the group receiving 1 mg/kg bortezomib+5 mg/kg xylitol. A “*” mark was added to a measurement that was determined to have a significant difference from that of the control group in a test, using a significance level of 1% (in the figure, shown as “*P<0.01 vs control group”).

In the group receiving 1 mg/kg bortezomib (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg xylitol (dotted line with white squares), which received only xylitol, and the group receiving 1 mg/kg bortezomib+5 mg/kg xylitol (broken line with black squares), which received bortezomib and xylitol in combination, exhibited a withdrawal response time (latent time) approximately similar to that of the control group (solid line with white circles). The two groups receiving xylitol also did not show a reduction in the withdrawal response time thereafter. In these two groups, the reduction in the withdrawal response time (latent time) was suppressed compared with the group receiving 1 mg/kg bortezomib (broken line with black circles).

(12) Von Frey Test

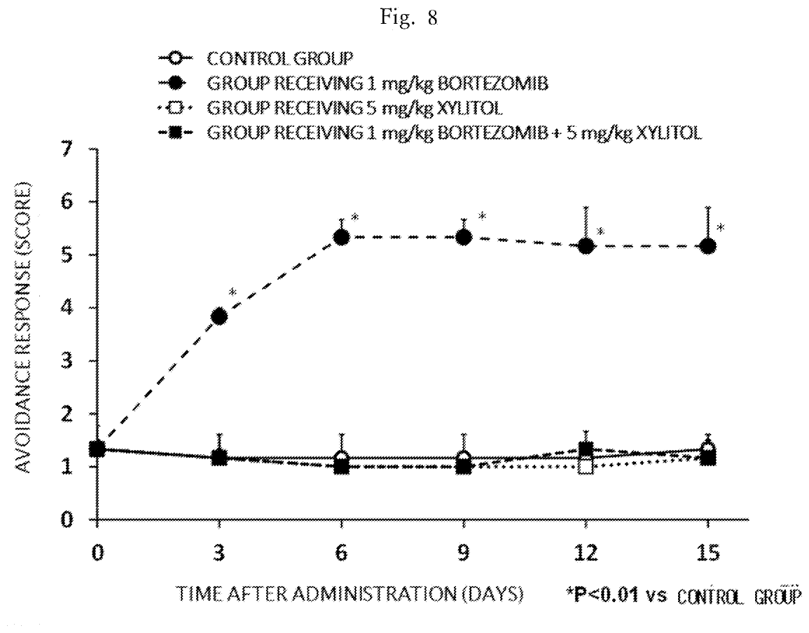

The mice in the four groups described in (10) of the present example (Example 1) were placed in a cage, and the avoidance response frequency (score) was measured by pressing a filament with a bending force of 0.16 g against the back of the hind paw. The results are shown in FIG. 8.

With reference to FIG. 8, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 1 mg/kg bortezomib; the dotted line with white squares represents the group receiving 5 mg/kg xylitol; and the broken line with black squares represents the group receiving 1 mg/kg bortezomib+5 mg/kg xylitol. A “*” mark was added to a measurement that was determined to have a significant difference from that of the control group in a test, using a significance level of 1% (in the figure, shown as “*P<0.01 vs control group”).

The group receiving 1 mg/kg bortezomib (broken line with black circles) exhibited a remarkable increase in the avoidance response score compared with the control group (solid line with white circles). On the other hand, the group receiving 5 mg/kg xylitol (dotted line with white squares), which received only xylitol, and the group receiving 1 mg/kg bortezomib+5 mg/kg xylitol (broken line with black squares), which received xylitol in combination, exhibited avoidance response scores similar to that of the control group (solid line with white circles) throughout the experimental period. In these two groups, an increase in the avoidance response score was suppressed compared with the group receiving 1 mg/kg bortezomib (broken line with black circles).

Both the cold plate test (FIG. 7) and the von Frey test (FIG. 8) demonstrated that the group receiving 1 mg/kg bortezomib (broken line with black circles) exhibited a significant change in both the withdrawal response time and the avoidance response score compared with the control group (solid line with white circles), the change being a shift to a greater degree of shunning the stimulus. This demonstrates that the administration of bortezomib caused peripheral neuropathy (peripheral nerve hypersensitivity) to develop.

On the other hand, the group receiving 5 mg/kg xylitol (dotted line with white squares) and the group receiving 1 mg/kg bortezomib+5 mg/kg xylitol (broken line with black squares), which received xylitol, showed results similar to those of the control group. Therefore, it can be concluded that xylitol suppresses the peripheral neuropathy (peripheral nerve hypersensitivity) induced by bortezomib. In other words, xylitol serves as a preventive composition (preventive agent) for peripheral neuropathy that is caused by bortezomib (anticancer drug).

### (Example 2) Xylitol-Containing Therapeutic Agent for Peripheral Neuropathy that is Caused by Administration of Anticancer Drug

### <Therapeutic Action of Xylitol on Peripheral Neuropathy in a Mouse Caused by Oxaliplatin>

It was found that xylitol is capable of preventing the peripheral neuropathy that is caused by oxaliplatin. Therefore, whether xylitol had a therapeutic action of alleviating peripheral neuropathy after the development of peripheral neuropathy via the intake of an anticancer drug was subsequently examined.

(1) Administration of Test Article

Six to seven-week-old female Balb/c mice were used for the tests as with Example 1. All the mice were acclimated for seven days after arrival. Then, the mice were grouped into the following five groups: a control group, a group receiving only oxaliplatin, and three groups receiving oxaliplatin and three different doses of xylitol. Each group consisted of seven mice.

On Day 8 of acclimation, the mice in the groups other than the control group were intraperitoneally administered with oxaliplatin at a dose of 6 mg/kg. This day was designated as the first day of the administration (Day 0). Thereafter, these mice were intraperitoneally administered with oxaliplatin at the same dose on Day 7 and Day 14 for a total of 3 doses.

The mice in a first group receiving oxaliplatin+xylitol were orally administered with xylitol at a dose of 5 mg/kg daily from Day 6 of administration. The mice in a second group receiving oxaliplatin+xylitol were orally administered with xylitol at a dose of 25 mg/kg daily from Day 6 of administration. The mice in a third group receiving oxaliplatin+xylitol were orally administered with xylitol at a dose of 100 mg/kg daily from Day 6 of administration. In this context, “Day 6 of administration” means that the number of days after administration is 6. The same applies hereinafter.

These respective five groups are referred to as the control group, the group receiving 6 mg/kg oxaliplatin, the group receiving 6 mg/kg oxaliplatin+5 mg/kg xylitol, the group receiving 6 mg/kg oxaliplatin+25 mg/kg xylitol, and the group receiving 6 mg/kg oxaliplatin+100 mg/kg xylitol.

(2) Cold Plate Test

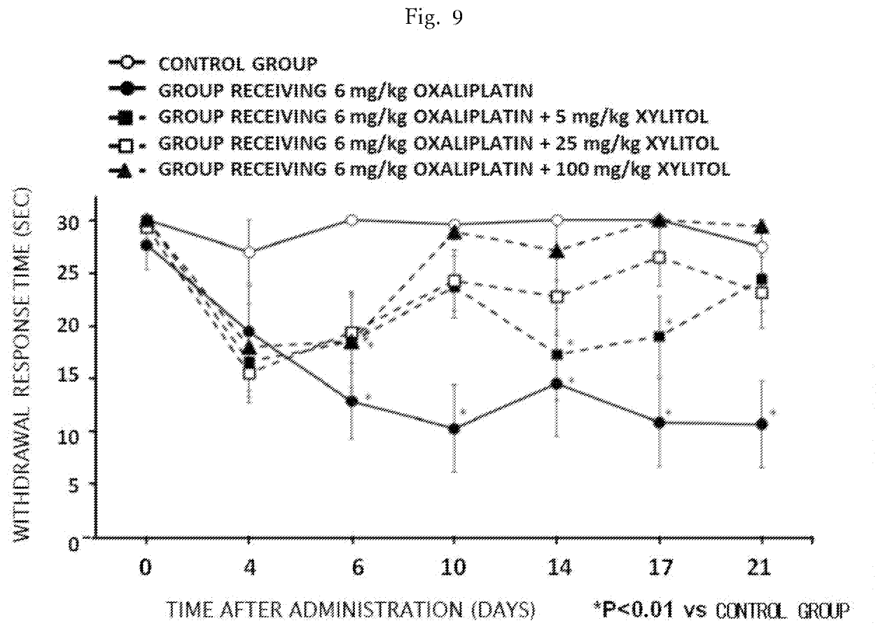

A test was performed on the mice in the five groups described in (1) of the present example (Example 2) to examine the effect of xylitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 9.

With reference to FIG. 9, the horizontal axis represents the time after administration (days), and the vertical axis represents the average value of the withdrawal response time (seconds) of the mice in each group. The solid line with white circles represents the control group; the solid line with black circles represents the group receiving 6 mg/kg oxaliplatin; the broken line with black squares represents the group receiving 6 mg/kg oxaliplatin+5 mg/kg xylitol; the broken line with white squares represents the group receiving 6 mg/kg oxaliplatin+25 mg/kg xylitol; and the broken line with black triangles represents the group receiving 6 mg/kg oxaliplatin+100 mg/kg xylitol. A “*” mark was added to a measurement that was determined to have a significant difference from that of the control group in a test, using a significance level of 1% (in the figure, shown as “*P<0.01 vs control group”).

With reference to FIG. 9, in all of the four groups receiving oxaliplatin other than the control group, a uniform reduction in the withdrawal response time (latent time) was observed by Day 6 after administration. However, the latent time tended to become longer from Day 6 onwards in the following three groups receiving xylitol: the group receiving 6 mg/kg oxaliplatin+5 mg/kg xylitol, the group receiving 6 mg/kg oxaliplatin+25 mg/kg xylitol, and the group receiving 6 mg/kg oxaliplatin+100 mg/kg xylitol. On Day 10, these three groups and the control group had a significantly longer withdrawal response time (latent time) compared with the group receiving 6 mg/kg oxaliplatin.

Furthermore, in the group receiving 6 mg/kg oxaliplatin+5 mg/kg xylitol, on Day 14 and Day 17, no significant difference from the group receiving 6 mg/kg oxaliplatin was observed, but on Day 21 of administration, a significantly longer withdrawal response time (latent time) was observed.

In the group receiving 6 mg/kg oxaliplatin+25 mg/kg xylitol, an increase in the withdrawal response time (latent time) was observed from Day 6 of xylitol administration. In the group receiving 6 mg/kg oxaliplatin+100 mg/kg xylitol, the withdrawal response time (latent time) from Day 10 onwards changed over time in a similar manner to the control group.

The withdrawal response time (latent time) was greatly reduced on Day 6 of administration in the four groups receiving oxaliplatin (the group receiving 6 mg/kg oxaliplatin, the group receiving 6 mg/kg oxaliplatin+5 mg/kg xylitol, the group receiving 6 mg/kg oxaliplatin+25 mg/kg xylitol, and the group receiving 6 mg/kg oxaliplatin+100 mg/kg xylitol). Thus, it is presumed that the mice in these groups developed peripheral neuropathy (peripheral nerve hypersensitivity).

After that, all of the three groups receiving xylitol exhibited a significantly longer withdrawal response time (latent time) compared with the group receiving 6 mg/kg oxaliplatin and no xylitol. This result signifies that the peripheral neuropathy (peripheral nerve hypersensitivity) that had developed beforehand was ameliorated by taking xylitol. Thus, it was found that xylitol also serves as a therapeutic composition (therapeutic agent) for peripheral neuropathy (peripheral nerve hypersensitivity).

(4) Administration of Test Article

Six to seven-week-old female Balb/c mice were used for the tests as with Example 1. All the mice were acclimated for seven days after arrival. Then, the mice were grouped into the following four groups: a control group, a group receiving oxaliplatin, a group receiving xylitol, and a group receiving oxaliplatin and xylitol (a group receiving oxaliplatin+xylitol). Each group consisted of five mice.

On Day 8 of acclimation, the mice in the group receiving oxaliplatin and the group receiving oxaliplatin+xylitol were intraperitoneally administered with oxaliplatin at a dose of 6 mg/kg. This day was designated as the first day of the administration (Day 0). Thereafter, these mice were intraperitoneally administered with oxaliplatin at the same dose on Day 7 and Day 14 for a total of 3 doses.

The mice in the group receiving 100 mg/kg xylitol were orally administered with xylitol at a dose of 100 mg/kg daily from Day 0. The mice in the group receiving oxaliplatin+100 mg/kg xylitol were orally administered with xylitol at a dose of 100 mg/kg daily from Day 6 of administration.

(5) Von Frey Test

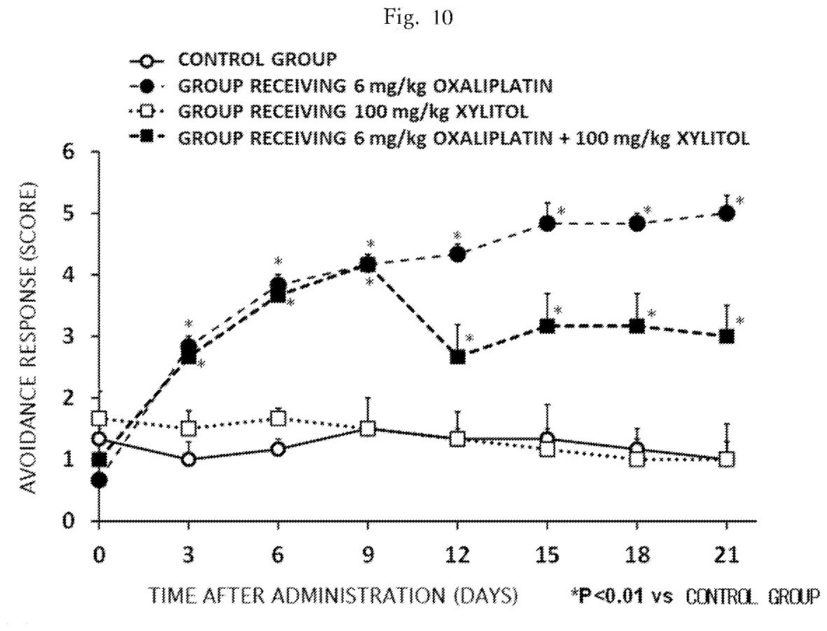

For the mice in the four groups described in (4) of the present example (Example 2), the avoidance response frequency (score) was measured by pressing a filament with a bending force of 0.16 g against the back of the hind paw. The results are shown in FIG. 10.

With reference to FIG. 10, the horizontal axis represents the time after administration (days), and the vertical axis represents the average value of the avoidance responses (score) of the mice in each group. The solid line with white circles represents the control group; the broken line with black circles represents the group receiving 6 mg/kg oxaliplatin; the dotted line with white squares represents the group receiving 100 mg/kg xylitol; and the broken line with black squares represents the group receiving 6 mg/kg oxaliplatin+100 mg/kg xylitol. A “*” mark was added to a measurement that was determined to have a significant difference from that of the control group in a test, using a significance level of 1% (in the figure, shown as “*P<0.01 vs control group”).

The group receiving oxaliplatin (broken line with black circles) exhibited a remarkable increase in the avoidance response score compared with the control group (solid line with white circles). Thus, it is presumed that the mice in the group receiving oxaliplatin developed peripheral neuropathy (peripheral nerve hypersensitivity). On the other hand, the group receiving 6 mg/kg oxaliplatin+100 mg/kg xylitol (broken line with black squares), which received xylitol in combination, maintained a score clearly lower than that of the group receiving oxaliplatin (broken line with black circles) from Day 12 onwards. Thus, it was found that xylitol also serves as a therapeutic composition (therapeutic agent) for peripheral neuropathy (peripheral nerve hypersensitivity).

<Therapeutic Action of Xylitol on Peripheral Neuropathy in a Mouse Caused by Paclitaxel>

It was found that xylitol is capable of preventing the peripheral neuropathy that is caused by paclitaxel. Therefore, whether xylitol had a therapeutic action of alleviating peripheral neuropathy after the development of peripheral neuropathy via the intake of an anticancer drug was subsequently examined.

(6) Administration of Test Article

Six to seven-week-old female Balb/c mice were used for the tests as with Example 1. All the mice were acclimated for seven days after arrival. Then, the mice were grouped into the following four groups: a control group, a group receiving paclitaxel, a group receiving xylitol, and a group receiving paclitaxel and xylitol (a group receiving paclitaxel+xylitol). Each group consisted of five mice.

On Day 8 of acclimation, the mice in the group receiving paclitaxel and the group receiving paclitaxel+xylitol were intraperitoneally administered with paclitaxel at a dose of 6 mg/kg. This day was designated as the first day of the administration (Day 0). Thereafter, these mice were intraperitoneally administered with paclitaxel at the same dose on Day 7 and Day 14 for a total of 3 doses.

The mice in the group receiving 100 mg/kg xylitol were orally administered with xylitol at a dose of 100 mg/kg daily from Day 0. The mice in the group receiving paclitaxel+100 mg/kg xylitol were orally administered with xylitol at a dose of 100 mg/kg daily from Day 6 of administration.

(7) Cold Plate Test

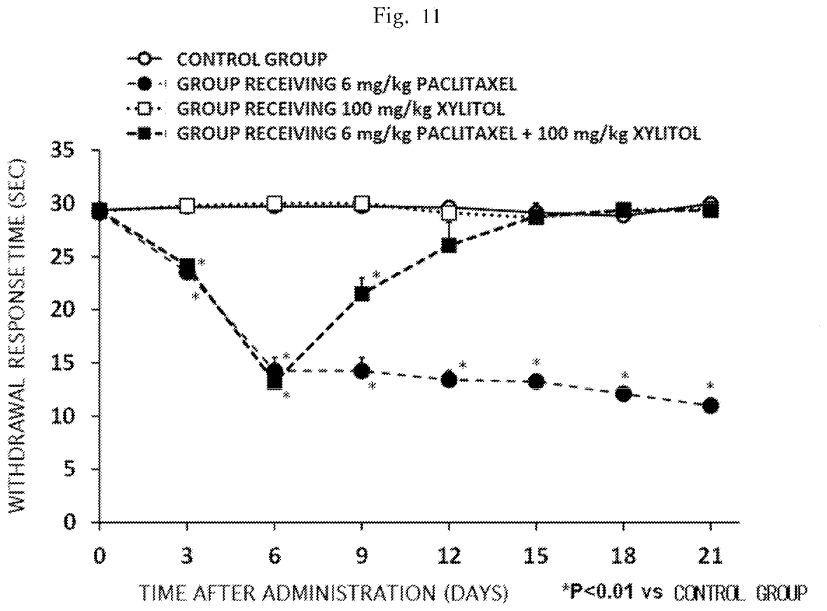

A test was performed on the mice in the four groups described in (6) of the present example (Example 2) to examine the effect of xylitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 11.

With reference to FIG. 11, the horizontal axis represents the time after administration (days), and the vertical axis represents the average value of the withdrawal response time (seconds) of the mice in each group. The solid line with white circles represents the control group; the broken line with black circles represents the group receiving 6 mg/kg paclitaxel; the dotted line with white squares represents the group receiving 100 mg/kg xylitol; and the broken line with black squares represents the group receiving 6 mg/kg paclitaxel+100 mg/kg xylitol. A “*” mark was added to a measurement that was determined to have a significant difference from that of the control group in a test, using a significance level of 1% (in the figure, shown as “*P<0.01 vs control group”).

With reference to FIG. 11, in the two groups receiving paclitaxel other than the control group and the group receiving xylitol, a uniform reduction in the withdrawal response time (latent time) was observed by Day 6 after administration. However, the latent time tended to become longer from Day 6 onwards in the group receiving 6 mg/kg paclitaxel+100 mg/kg xylitol. On Day 12, the group receiving 6 mg/kg paclitaxel+100 mg/kg xylitol had a significantly longer withdrawal response time (latent time) compared with the group receiving 6 mg/kg paclitaxel.

The withdrawal response time (latent time) was greatly reduced on Day 6 of administration in the two groups receiving paclitaxel (the group receiving 6 mg/kg paclitaxel, and the group receiving 6 mg/kg paclitaxel+100 mg/kg xylitol). Thus, it is presumed that the mice in these groups developed peripheral neuropathy (peripheral nerve hypersensitivity).

After that, the groups receiving xylitol exhibited a significantly longer withdrawal response time (latent time) compared with the group receiving 6 mg/kg paclitaxel and no xylitol. This result signifies that the peripheral neuropathy (peripheral nerve hypersensitivity) that had developed beforehand was ameliorated by taking xylitol. Thus, it was found that xylitol also serves as a therapeutic composition (therapeutic agent) for peripheral neuropathy (peripheral nerve hypersensitivity).

(8) Von Frey Test

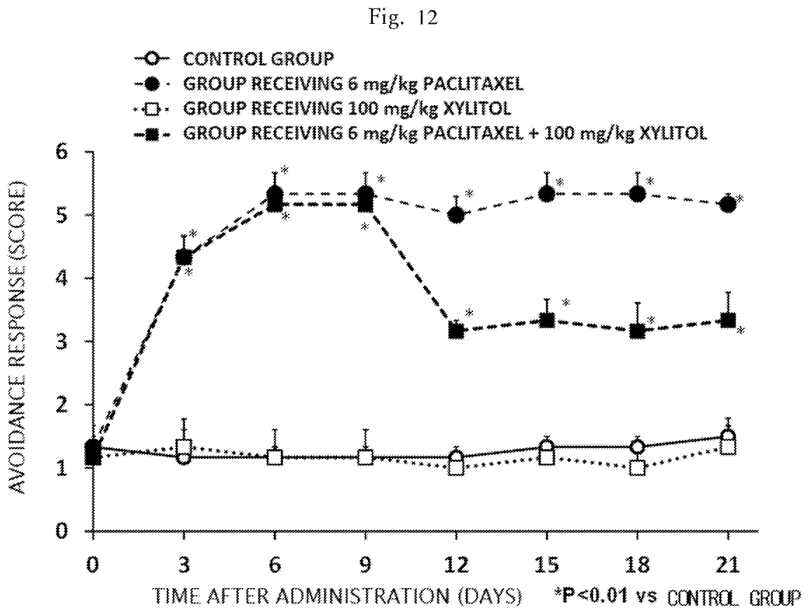

For the mice in the four groups described in (6) of the present example (Example 2), the avoidance response frequency (score) was measured by pressing a filament with a bending force of 0.16 g against the back of the hind paw. The results are shown in FIG. 12.

With reference to FIG. 12, the horizontal axis represents the time after administration (days), and the vertical axis represents the average value of the avoidance responses (score) of the mice in each group. The solid line with white circles represents the control group; the broken line with black circles represents the group receiving 6 mg/kg paclitaxel; the dotted line with white squares represents the group receiving 100 mg/kg xylitol; and the broken line with black squares represents the group receiving 6 mg/kg paclitaxel+100 mg/kg xylitol. A “*” mark was added to a measurement that was determined to have a significant difference from that of the control group in a test, using a significance level of 1% (in the figure, shown as “*P<0.01 vs control group”).

The group receiving paclitaxel (broken line with black circles) exhibited a remarkable increase in the avoidance response score compared with the control group (solid line with white circles). Thus, it is presumed that the mice in the group receiving paclitaxel developed peripheral neuropathy (peripheral nerve hypersensitivity). On the other hand, the group receiving 6 mg/kg paclitaxel+100 mg/kg xylitol (broken line with black squares), which received xylitol in combination, maintained a score clearly lower than that of the group receiving paclitaxel (broken line with black circles) from Day 12 onwards. Thus, it was found that xylitol also serves as a therapeutic composition (therapeutic agent) for peripheral neuropathy (peripheral nerve hypersensitivity).

<Therapeutic Action of Xylitol on Peripheral Neuropathy in a Mouse Caused by Vincristine>

It was found that xylitol is capable of preventing the peripheral neuropathy that is caused by vincristine. Therefore, whether xylitol had a therapeutic action of alleviating peripheral neuropathy after the development of peripheral neuropathy via the intake of an anticancer drug was subsequently examined.

(9) Administration of Test Article

Six to seven-week-old female Balb/c mice were used for the tests as with Example 1. All the mice were acclimated for seven days after arrival. Then, the mice were grouped into the following four groups: a control group, a group receiving vincristine, a group receiving xylitol, and a group receiving vincristine and xylitol (a group receiving vincristine+xylitol). Each group consisted of five mice.

On Day 8 of acclimation, the mice in the group receiving vincristine and the group receiving vincristine+xylitol were intraperitoneally administered with vincristine at a dose of 0.2 mg/kg. This day was designated as the first day of the administration (Day 0). Thereafter, these mice were intraperitoneally administered with vincristine at the same dose on Day 7 and Day 14 for a total of 3 doses.

The mice in the group receiving 100 mg/kg xylitol were orally administered with xylitol at a dose of 100 mg/kg daily from Day 0. The mice in the group receiving vincristine+100 mg/kg xylitol were orally administered with xylitol at a dose of 100 mg/kg daily from Day 6 of administration.

(10) Cold Plate Test

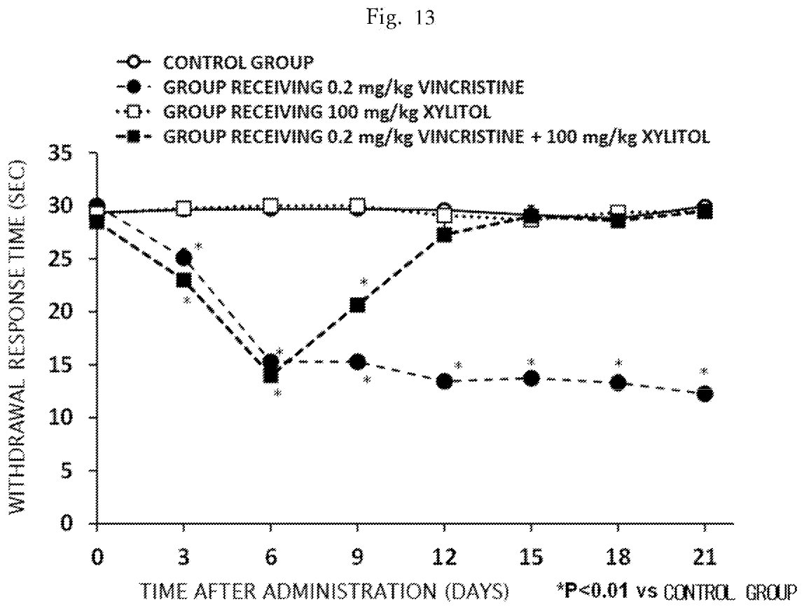

A test was performed on the mice in the four groups described in (9) of the present example (Example 2) to examine the effect of xylitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 13.

With reference to FIG. 13, the horizontal axis represents the time after administration (days), and the vertical axis represents the average value of the withdrawal response time (seconds) of the mice in each group. The solid line with white circles represents the control group; the broken line with black circles represents the group receiving 0.2 mg/kg vincristine; the dotted line with white squares represents the group receiving 100 mg/kg xylitol; and the broken line with black squares represents the group receiving 0.2 mg/kg vincristine+100 mg/kg xylitol. A “*” mark was added to a measurement that was determined to have a significant difference from that of the control group in a test, using a significance level of 1% (in the figure, shown as “*P<0.01 vs control group”).

With reference to FIG. 13, in the two groups receiving vincristine other than the control group and the group receiving xylitol, a uniform reduction in the withdrawal response time (latent time) was observed by Day 6 after administration. However, the latent time tended to become longer from Day 6 onwards in the group receiving 0.2 mg/kg vincristine+100 mg/kg xylitol. On Day 12, the group receiving 0.2 mg/kg vincristine+100 mg/kg xylitol had a significantly longer withdrawal response time (latent time) compared with the group receiving 0.2 mg/kg vincristine.

The withdrawal response time (latent time) was greatly reduced on Day 6 of administration in the two groups receiving vincristine (the group receiving 0.2 mg/kg vincristine, and the group receiving 0.2 mg/kg vincristine+100 mg/kg xylitol). Thus, it is presumed that the mice in these groups developed peripheral neuropathy (peripheral nerve hypersensitivity).

After that, the groups receiving xylitol exhibited a significantly longer withdrawal response time (latent time) compared with the group receiving 0.2 mg/kg vincristine and no xylitol. This result signifies that the peripheral neuropathy (peripheral nerve hypersensitivity) that had developed beforehand was ameliorated by taking xylitol. Thus, it was found that xylitol also serves as a therapeutic composition (therapeutic agent) for peripheral neuropathy (peripheral nerve hypersensitivity).

(11) Von Frey Test

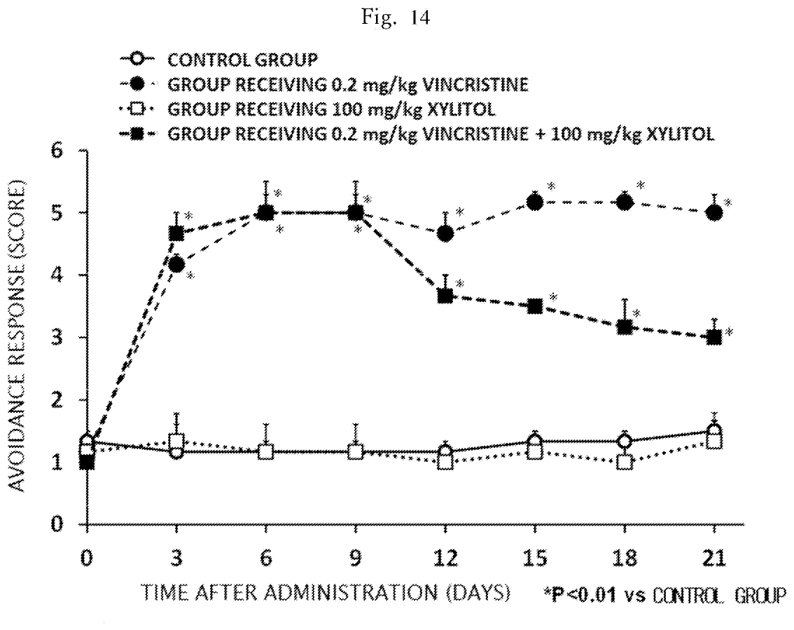

For the mice in the four groups described in (9) of the present example (Example 2), the avoidance response frequency (score) was measured by pressing a filament with a bending force of 0.16 g against the back of the hind paw. The results are shown in FIG. 14.

With reference to FIG. 14, the horizontal axis represents the time after administration (days), and the vertical axis represents the average value of the avoidance responses (score) of the mice in each group. The solid line with white circles represents the control group; the broken line with black circles represents the group receiving 0.2 mg/kg vincristine; the dotted line with white squares represents the group receiving 100 mg/kg xylitol; and the broken line with black squares represents the group receiving 0.2 mg/kg vincristine+100 mg/kg xylitol. A “*” mark was added to a measurement that was determined to have a significant difference from that of the control group in a test, using a significance level of 1% (in the figure, shown as “*P<0.01 vs control group”).

The group receiving vincristine (broken line with black circles) exhibited a remarkable increase in the avoidance response score compared with the control group (solid line with white circles). Thus, it is presumed that the mice in the group receiving vincristine developed peripheral neuropathy (peripheral nerve hypersensitivity). On the other hand, the group receiving 0.2 mg/kg vincristine+100 mg/kg xylitol (broken line with black squares), which received xylitol in combination, maintained a score clearly lower than that of the group receiving vincristine (broken line with black circles) from Day 12 onwards. Thus, it was found that xylitol also serves as a therapeutic composition (therapeutic agent) for peripheral neuropathy (peripheral nerve hypersensitivity).

<Therapeutic Action of Xylitol on Peripheral Neuropathy in a Mouse Caused by Bortezomib>

It was found that xylitol is capable of preventing the peripheral neuropathy that is caused by bortezomib. Therefore, whether xylitol had a therapeutic action of alleviating peripheral neuropathy after the development of peripheral neuropathy via the intake of an anticancer drug was subsequently examined.

(12) Administration of Test Article

Six to seven-week-old female Balb/c mice were used for the tests as with Example 1. All the mice were acclimated for seven days after arrival. Then, the mice were grouped into the following four groups: a control group, a group receiving bortezomib, a group receiving xylitol, and a group receiving bortezomib and xylitol (a group receiving bortezomib+xylitol). Each group consisted of five mice.

On Day 8 of acclimation, the mice in the group receiving bortezomib and the group receiving bortezomib+xylitol were intraperitoneally administered with bortezomib at a dose of 1 mg/kg. This day was designated as the first day of the administration (Day 0). Thereafter, these mice were intraperitoneally administered with bortezomib at the same dose on Day 7 and Day 14 for a total of 3 doses.

The mice in the group receiving 100 mg/kg xylitol were orally administered with xylitol at a dose of 100 mg/kg daily from Day 0. The mice in the group receiving bortezomib+100 mg/kg xylitol were orally administered with xylitol at a dose of 100 mg/kg daily from Day 6 of administration.

(13) Cold Plate Test

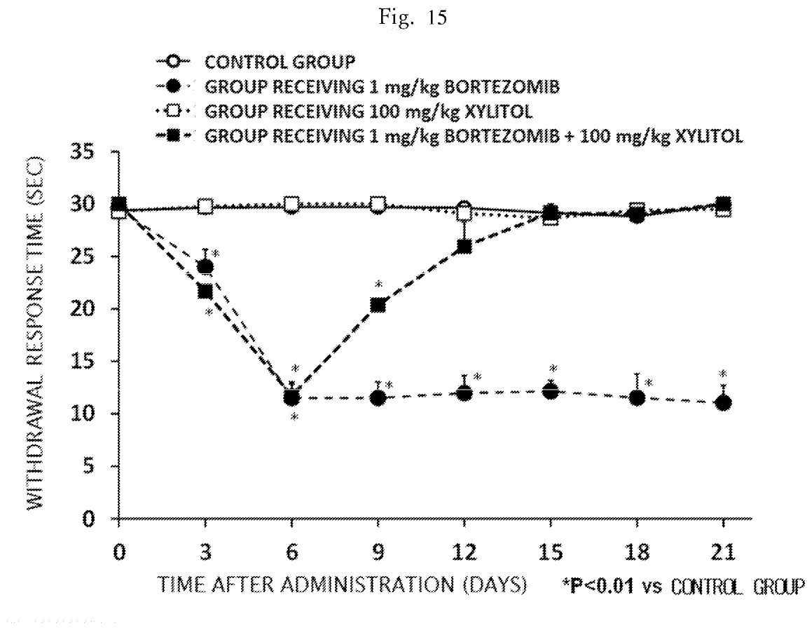

A test was performed on the mice in the four groups described in (12) of the present example (Example 2) to examine the effect of xylitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 15.

With reference to FIG. 15, the horizontal axis represents the time after administration (days), and the vertical axis represents the average value of the withdrawal response time (seconds) of the mice in each group. The solid line with white circles represents the control group; the broken line with black circles represents the group receiving 1 mg/kg bortezomib; the dotted line with white squares represents the group receiving 100 mg/kg xylitol; and the broken line with black squares represents the group receiving 1 mg/kg bortezomib+100 mg/kg xylitol. A “*” mark was added to a measurement that was determined to have a significant difference from that of the control group in a test, using a significance level of 1% (in the figure, shown as “*P<0.01 vs control group”).

With reference to FIG. 15, in the two groups receiving bortezomib other than the control group and the group receiving xylitol, a uniform reduction in the withdrawal response time (latent time) was observed by Day 6 after administration. However, the latent time tended to become longer from Day 6 onwards in the group receiving 1 mg/kg bortezomib+100 mg/kg xylitol. On Day 12, the group receiving 1 mg/kg bortezomib+100 mg/kg xylitol had a significantly longer withdrawal response time (latent time) compared with the group receiving 1 mg/kg bortezomib.

The withdrawal response time (latent time) was greatly reduced on Day 6 of administration in the two groups receiving bortezomib (the group receiving 1 mg/kg bortezomib, and the group receiving 1 mg/kg bortezomib+100 mg/kg xylitol). Thus, it is presumed that the mice in these groups developed peripheral neuropathy (peripheral nerve hypersensitivity).

After that, the groups receiving xylitol exhibited a significantly longer withdrawal response time (latent time) compared with the group receiving 1 mg/kg bortezomib and no xylitol. This result signifies that the peripheral neuropathy (peripheral nerve hypersensitivity) that had developed beforehand was ameliorated by taking xylitol. Thus, it was found that xylitol also serves as a therapeutic composition (therapeutic agent) for peripheral neuropathy (peripheral nerve hypersensitivity).

(14) Von Frey Test

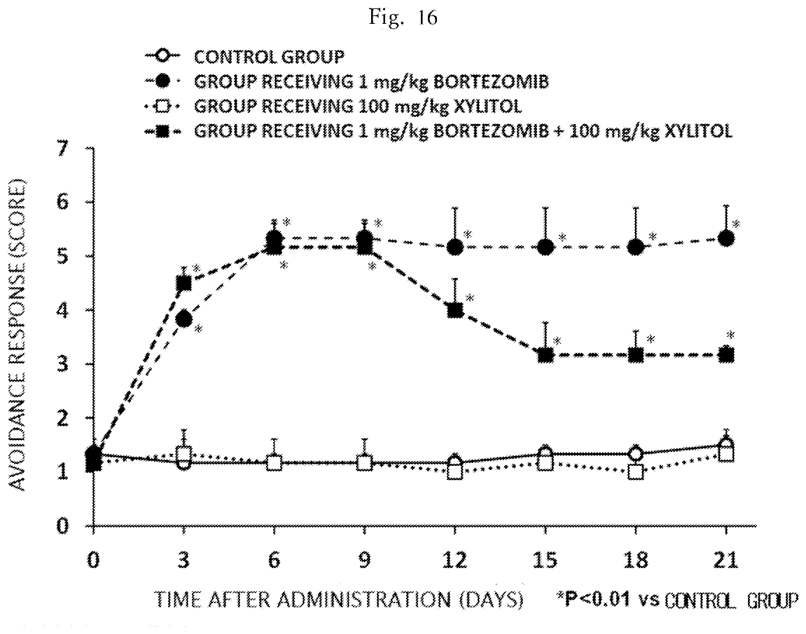

For the mice in the four groups described in (12) of the present example (Example 2), the avoidance response frequency (score) was measured by pressing a filament with a bending force of 0.16 g against the back of the hind paw. The results are shown in FIG. 16.

With reference to FIG. 16, the horizontal axis represents the time after administration (days), and the vertical axis represents the average value of the avoidance responses (score) of the mice in each group. The solid line with white circles represents the control group; the broken line with black circles represents the group receiving 1 mg/kg bortezomib; the dotted line with white squares represents the group receiving 100 mg/kg xylitol; and the broken line with black squares represents the group receiving 1 mg/kg bortezomib+100 mg/kg xylitol. A “*” mark was added to a measurement that was determined to have a significant difference from that of the control group in a test, using a significance level of 1% (in the figure, shown as “*P<0.01 vs control group”).

The group receiving bortezomib (broken line with black circles) exhibited a remarkable increase in the avoidance response score compared with the control group (solid line with white circles). Thus, it is presumed that the mice in the group receiving bortezomib developed peripheral neuropathy (peripheral nerve hypersensitivity). On the other hand, the group receiving 1 mg/kg bortezomib+100 mg/kg xylitol (broken line with black squares), which received xylitol in combination, maintained a score clearly lower than that of the group receiving bortezomib (broken line with black circles) from Day 12 onwards. Thus, it was found that xylitol also serves as a therapeutic composition (therapeutic agent) for peripheral neuropathy (peripheral nerve hypersensitivity).

### (Example 3) D-Threitol-Containing Preventive Agent for Peripheral Neuropathy that is Caused by Administration of Anticancer Drug

### <Preventive Action of D-Threitol on Peripheral Neuropathy in a Mouse Caused by Oxaliplatin>

The preventive action of D-threitol on hyperesthesia that occurs when an anticancer drug, oxaliplatin, was administered was examined. Examples of hyperesthesia include paresthesia that is caused by a low temperature stimulus and allodynia (severe pain induced by a tactile stimulus that does not usually cause pain) that is caused by a mechanical stimulus. D-threitol was orally administered to a mouse simultaneously with the administration of oxaliplatin, and the following tests (cold plate test and von Frey test) were performed.

(1) Administration of Test Article

Six to seven-week-old female Balb/c mice were used for the tests as with Example 1. All the mice were acclimated for seven days after arrival. Then, the mice were grouped into the following four groups: a control group, a group receiving oxaliplatin, a group receiving D-threitol, and a group receiving oxaliplatin and D-threitol (a group receiving oxaliplatin+D-threitol). Each group consisted of five mice.

On Day 8 of acclimation, the mice in the group receiving oxaliplatin and the group receiving oxaliplatin+D-threitol were intraperitoneally administered with oxaliplatin at a dose of 6 mg/kg. This day was designated as the first day of the administration (Day 0). Thereafter, these mice were intraperitoneally administered with oxaliplatin at a daily dose of 6 mg/kg on Day 7 and Day 14.

The mice in the group receiving D-threitol and the group receiving oxaliplatin+D-threitol were orally administered with D-threitol at a dose of 5 mg/kg daily from Day 0.

These respective groups are referred to as the control group, the group receiving 6 mg/kg oxaliplatin, the group receiving 5 mg/kg D-threitol, and the group receiving 6 mg/kg oxaliplatin+5 mg/kg D-threitol.

(2) Cold Plate Test

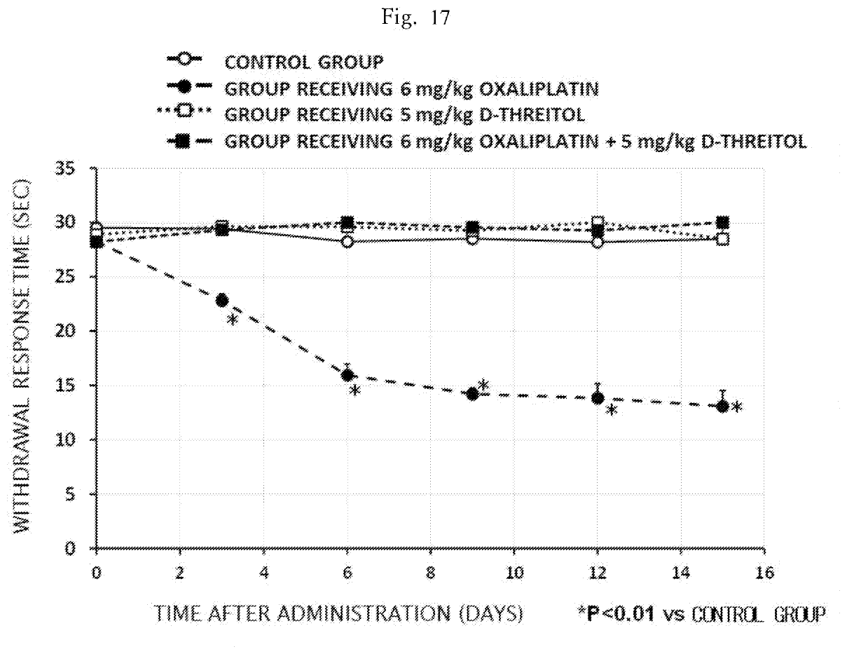

A cold plate test was performed to examine the effect of D-threitol on paresthesia that is caused by a low temperature stimulus. The mice in the four groups described in (1) of the present example (Example 3) were placed on a cold plate set at 10° C., and withdrawal response time (latent time) was measured. It is presumed that a shorter latent time reflects a greater degree of shunning the low temperature stimulus by the cold plate. The results are shown in FIG. 17.

With reference to FIG. 17, the horizontal axis represents the time after administration (days), and the vertical axis represents the average value of the withdrawal response time (seconds) of the mice in each group. The solid line with white circles represents the control group; the broken line with black circles represents the group receiving 6 mg/kg oxaliplatin; the dotted line with white squares represents the group receiving 5 mg/kg D-threitol; and the broken line with black squares represents the group receiving 6 mg/kg oxaliplatin+5 mg/kg D-threitol. A “*” mark was added to a measurement that was determined to have a significant difference from that of the control group in a test, using a significance level of 1% (in the figure, shown as “*P<0.01 vs control group”).

In the group receiving 6 mg/kg oxaliplatin (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg D-threitol (dotted line with white squares), which received only D-threitol, and the group receiving 6 mg/kg oxaliplatin+5 mg/kg D-threitol (broken line with black squares), which received oxaliplatin and D-threitol in combination, exhibited a withdrawal response time (latent time) approximately similar to that of the control group (solid line with white circles). The two groups receiving D-threitol also did not show a reduction in the withdrawal response time thereafter. In these two groups, the reduction in the withdrawal response time (latent time) was suppressed compared with the group receiving 6 mg/kg oxaliplatin (broken line with black circles).

(3) Von Frey Test

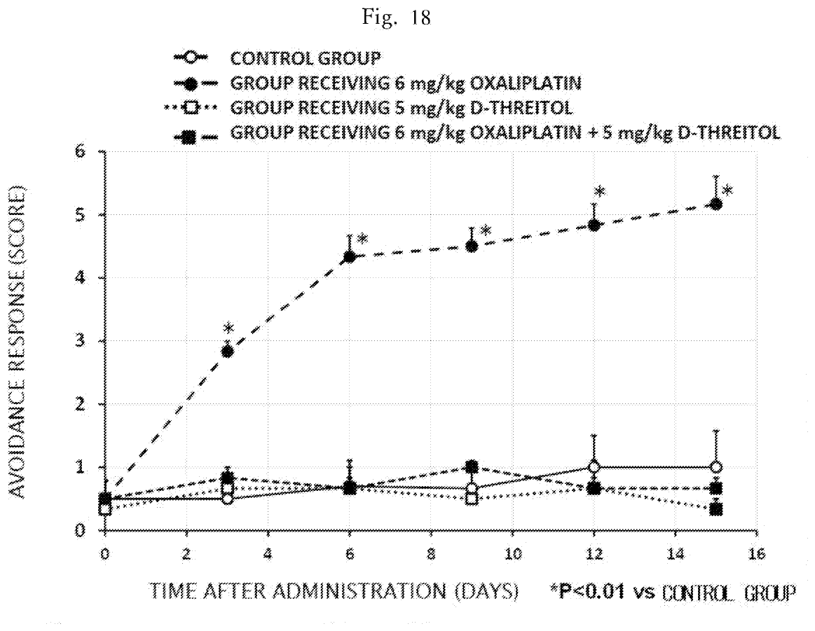

The mice in the four groups described in (1) of the present example (Example 3) were placed in a cage, and the avoidance response frequency (score) was measured by pressing a filament with a bending force of 0.16 g against the back of the hind paw. The results are shown in FIG. 18.

With reference to FIG. 18, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 6 mg/kg oxaliplatin; the dotted line with white squares represents the group receiving 5 mg/kg D-threitol; and the broken line with black squares represents the group receiving 6 mg/kg oxaliplatin+5 mg/kg D-threitol. A “*” mark was added to a measurement that was determined to have a significant difference from that of the control group in a test, using a significance level of 1% (in the figure, shown as “*P<0.01 vs control group”).

The group receiving 6 mg/kg oxaliplatin (broken line with black circles) exhibited a remarkable increase in the avoidance response score compared with the control group (solid line with white circles). On the other hand, the group receiving 5 mg/kg D-threitol (dotted line with white squares) and the group receiving 6 mg/kg oxaliplatin+5 mg/kg D-threitol (broken line with black squares), which received D-threitol in combination, exhibited avoidance response scores similar to that of the control group (solid line with white circles) throughout the experimental period. In these two groups, an increase in the avoidance response score was suppressed compared with the group receiving 6 mg/kg oxaliplatin (broken line with black circles).

Both the cold plate test (FIG. 17) and the von Frey test (FIG. 18) demonstrated that the group receiving 6 mg/kg oxaliplatin (broken line with black circles) exhibited a significant change in both the withdrawal response time and the avoidance response score compared with the control group (solid line with white circles), the change being a shift to a greater degree of shunning the stimulus. This demonstrates that the administration of oxaliplatin caused peripheral neuropathy (peripheral nerve hypersensitivity) to develop.

On the other hand, the group receiving 5 mg/kg D-threitol (dotted line with white squares) and the group receiving 6 mg/kg oxaliplatin+5 mg/kg D-threitol (broken line with black squares), which received D-threitol, showed results similar to those of the control group. Therefore, it can be concluded that D-threitol suppresses the peripheral neuropathy (peripheral nerve hypersensitivity) induced by oxaliplatin. In other words, D-threitol serves as a preventive composition (preventive agent) for peripheral neuropathy that is caused by oxaliplatin (anticancer drug).

<Preventive Action of D-Threitol on Peripheral Neuropathy in a Mouse Caused by Paclitaxel>

The preventive action of D-threitol on hyperesthesia that occurs when an anticancer drug, paclitaxel, was administered was examined. Examples of hyperesthesia include paresthesia that is caused by a low temperature stimulus and allodynia (severe pain induced by a tactile stimulus that does not usually cause pain) that is caused by a mechanical stimulus. D-threitol was orally administered to a mouse simultaneously with the administration of paclitaxel, and the following tests (cold plate test and von Frey test) were performed.

(4) Administration of Test Article

Six to seven-week-old female Balb/c mice were used for the tests as with Example 1. All the mice were acclimated for seven days after arrival. Then, the mice were grouped into the following four groups: a control group, a group receiving paclitaxel, a group receiving D-threitol, and a group receiving paclitaxel and D-threitol (a group receiving paclitaxel+D-threitol). Each group consisted of five mice.

On Day 8 of acclimation, the mice in the group receiving paclitaxel and the group receiving paclitaxel+D-threitol were intraperitoneally administered with paclitaxel at a dose of 6 mg/kg. This day was designated as Day 0 of administration. Thereafter, these mice were intraperitoneally administered with paclitaxel at a daily dose of 6 mg/kg on Day 7 and Day 14.

The mice in the group receiving D-threitol and the group receiving paclitaxel+D-threitol were orally administered with D-threitol at a dose of 5 mg/kg daily from Day 0.

These respective groups are referred to as the control group, the group receiving 6 mg/kg paclitaxel, the group receiving 5 mg/kg D-threitol, and the group receiving 6 mg/kg paclitaxel+5 mg/kg D-threitol.

(5) Cold Plate Test

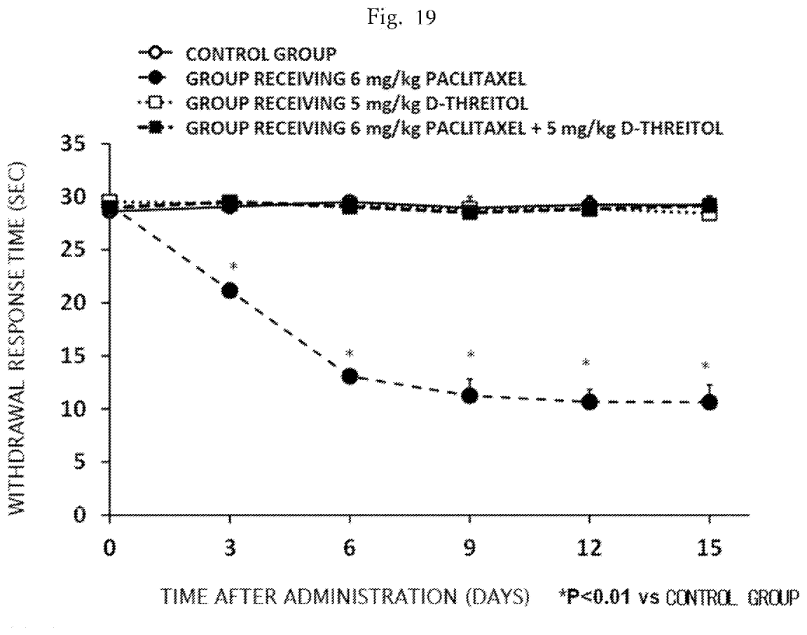

A cold plate test was performed to examine the effect of D-threitol on paresthesia that is caused by a low temperature stimulus. The mice in the four groups described in (4) of the present example (Example 3) were placed on a cold plate set at 10° C., and withdrawal response time (latent time) was measured. It is presumed that a shorter latent time reflects a greater degree of shunning the low temperature stimulus by the cold plate. The results are shown in FIG. 19.

With reference to FIG. 19, the horizontal axis represents the time after administration (days), and the vertical axis represents the average value of the withdrawal response time (seconds) of the mice in each group. The solid line with white circles represents the control group; the broken line with black circles represents the group receiving 6 mg/kg paclitaxel; the dotted line with white squares represents the group receiving 5 mg/kg D-threitol; and the broken line with black squares represents the group receiving 6 mg/kg paclitaxel+5 mg/kg D-threitol. A “*” mark was added to a measurement that was determined to have a significant difference from that of the control group in a test, using a significance level of 1% (in the figure, shown as “*P<0.01 vs control group”).

In the group receiving 6 mg/kg paclitaxel (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg D-threitol (dotted line with white squares), which received only D-threitol, and the group receiving 6 mg/kg paclitaxel+5 mg/kg D-threitol (broken line with black squares), which received paclitaxel and D-threitol in combination, exhibited a withdrawal response time (latent time) approximately similar to that of the control group (solid line with white circles). The two groups receiving D-threitol also did not show a reduction in the withdrawal response time thereafter. In these two groups, the reduction in the withdrawal response time (latent time) was suppressed compared with the group receiving 6 mg/kg paclitaxel (broken line with black circles).

(6) Von Frey Test

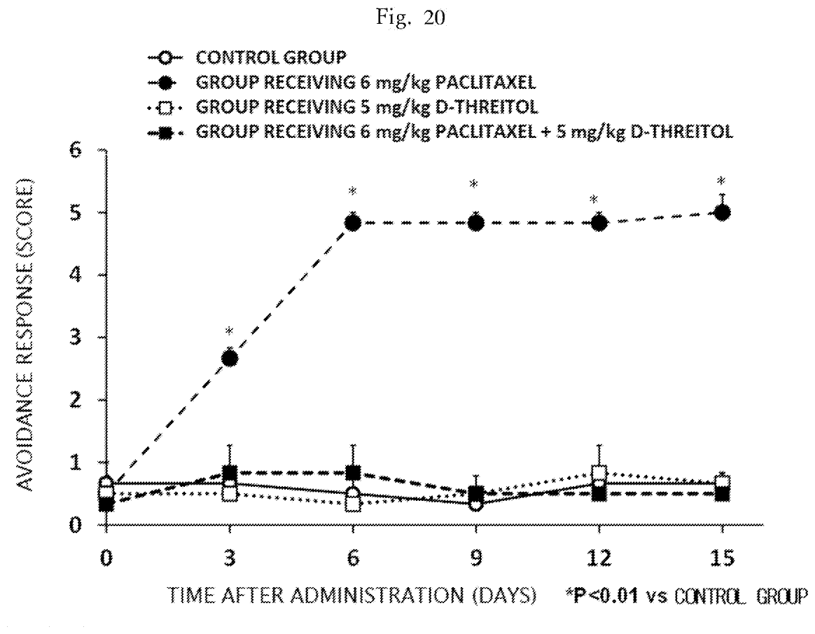

The mice in the four groups described in (4) of the present example (Example 3) were placed in a cage, and the avoidance response frequency (score) was measured by pressing a filament with a bending force of 0.16 g against the back of the hind paw. The results are shown in FIG. 20.

With reference to FIG. 20, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 6 mg/kg paclitaxel; the dotted line with white squares represents the group receiving 5 mg/kg D-threitol; and the broken line with black squares represents the group receiving 6 mg/kg paclitaxel+5 mg/kg D-threitol. A “*” mark was added to a measurement that was determined to have a significant difference from that of the control group in a test, using a significance level of 1% (in the figure, shown as “*P<0.01 vs control group”).

The group receiving 6 mg/kg paclitaxel (broken line with black circles) exhibited a remarkable increase in the avoidance response score compared with the control group (solid line with white circles). On the other hand, the group receiving 5 mg/kg D-threitol (dotted line with white squares), which received only D-threitol, and the group receiving 6 mg/kg paclitaxel+5 mg/kg D-threitol (broken line with black squares), which received D-threitol in combination, exhibited avoidance response scores similar to that of the control group (solid line with white circles) throughout the experimental period. In these two groups, an increase in the avoidance response score was suppressed compared with the group receiving 6 mg/kg paclitaxel (broken line with black circles).

Both the cold plate test (FIG. 19) and the von Frey test (FIG. 20) demonstrated that the group receiving 6 mg/kg paclitaxel (broken line with black circles) exhibited a significant change in both the withdrawal response time and the avoidance response score compared with the control group (solid line with white circles), the change being a shift to a greater degree of shunning the stimulus. This demonstrates that the administration of paclitaxel caused peripheral neuropathy (peripheral nerve hypersensitivity) to develop.

On the other hand, the group receiving 5 mg/kg D-threitol (dotted line with white squares) and the group receiving 6 mg/kg paclitaxel+5 mg/kg D-threitol (broken line with black squares), which received D-threitol, showed results similar to those of the control group. Therefore, it can be concluded that D-threitol suppresses the peripheral neuropathy (peripheral nerve hypersensitivity) induced by paclitaxel. In other words, D-threitol serves as a preventive composition (preventive agent) for peripheral neuropathy that is caused by paclitaxel (anticancer drug).

<Preventive Action of D-Threitol on Peripheral Neuropathy in a Mouse Caused by Vincristine>

The preventive action of D-threitol on hyperesthesia that occurs when an anticancer drug, vincristine, was administered was examined. Examples of hyperesthesia include paresthesia that is caused by a low temperature stimulus and allodynia (severe pain induced by a tactile stimulus that does not usually cause pain) that is caused by a mechanical stimulus. D-threitol was orally administered to a mouse simultaneously with the administration of vincristine, and the following tests (cold plate test and von Frey test) were performed.

(7) Administration of Test Article

Six to seven-week-old female Balb/c mice were used for the tests as with Example 1. All the mice were acclimated for seven days after arrival. Then, the mice were grouped into the following four groups: a control group, a group receiving vincristine, a group receiving D-threitol, and a group receiving vincristine and D-threitol (a group receiving vincristine+D-threitol). Each group consisted of five mice.

On Day 8 of acclimation, the mice in the group receiving vincristine and the group receiving vincristine+D-threitol were intraperitoneally administered with vincristine at a dose of 0.2 mg/kg. This day was designated as Day 0 of administration. Thereafter, these mice were intraperitoneally administered with vincristine at a daily dose of 0.2 mg/kg on Day 7 and Day 14.

The mice in the group receiving D-threitol and the group receiving vincristine+D-threitol were orally administered with D-threitol at a dose of 5 mg/kg daily from Day 0.

These respective groups are referred to as the control group, the group receiving 0.2 mg/kg vincristine, the group receiving 5 mg/kg D-threitol, and the group receiving 0.2 mg/kg vincristine+5 mg/kg D-threitol.

(8) Cold Plate Test

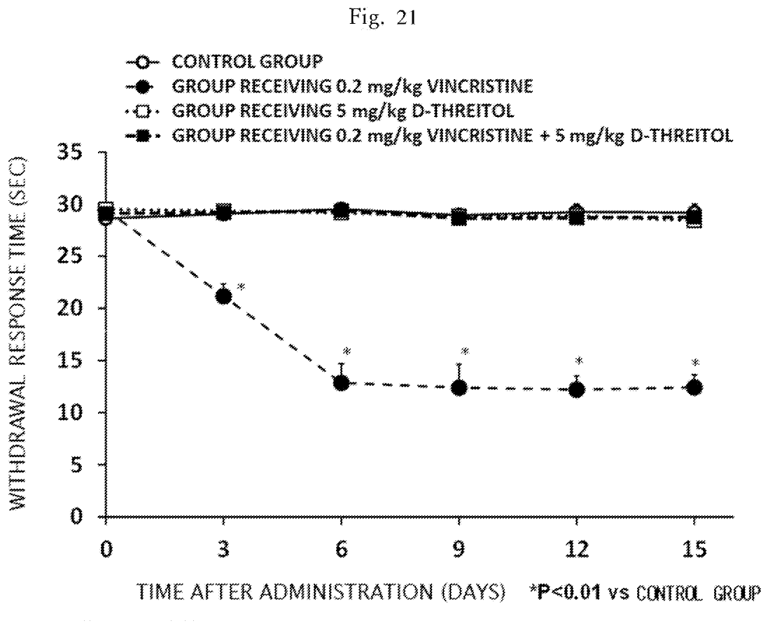

A cold plate test was performed to examine the effect of D-threitol on paresthesia that is caused by a low temperature stimulus. The mice in the four groups described in (7) of the present example (Example 3) were placed on a cold plate set at 10° C., and withdrawal response time (latent time) was measured. It is presumed that a shorter latent time reflects a greater degree of shunning the low temperature stimulus by the cold plate. The results are shown in FIG. 21.

With reference to FIG. 21, the horizontal axis represents the time after administration (days), and the vertical axis represents the average value of the withdrawal response time (seconds) of the mice in each group. The solid line with white circles represents the control group; the broken line with black circles represents the group receiving 0.2 mg/kg vincristine; the dotted line with white squares represents the group receiving 5 mg/kg D-threitol; and the broken line with black squares represents the group receiving 0.2 mg/kg vincristine+5 mg/kg D-threitol. A “*” mark was added to a measurement that was determined to have a significant difference from that of the control group in a test, using a significance level of 1% (in the figure, shown as “*P<0.01 vs control group”).

In the group receiving 0.2 mg/kg vincristine (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg D-threitol (dotted line with white squares), which received only D-threitol, and the group receiving 0.2 mg/kg vincristine+5 mg/kg D-threitol (broken line with black squares), which received vincristine and D-threitol in combination, exhibited a withdrawal response time (latent time) approximately similar to that of the control group (solid line with white circles). The two groups receiving D-threitol also did not show a reduction in the withdrawal response time thereafter. In these two groups, the reduction in the withdrawal response time (latent time) was suppressed compared with the group receiving 0.2 mg/kg vincristine (broken line with black circles).

(9) Von Frey Test

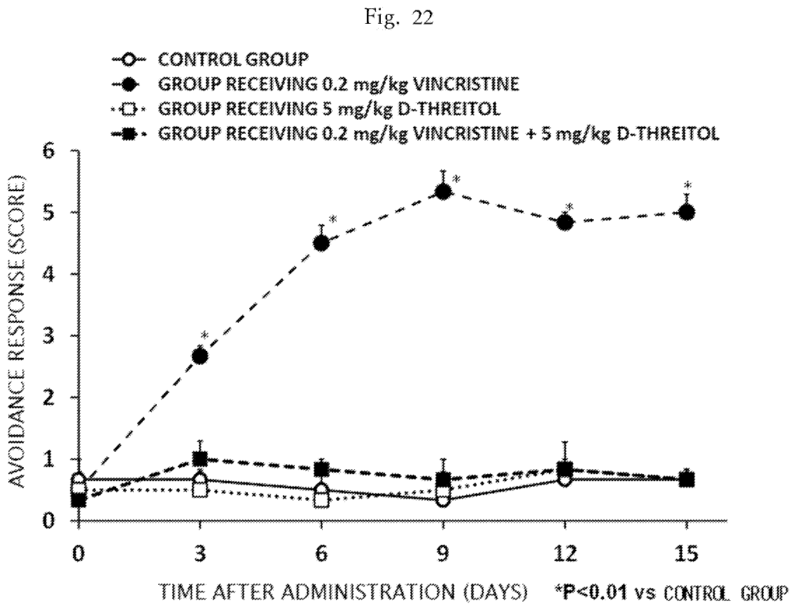

The mice in the four groups described in (7) of the present example (Example 3) were placed in a cage, and the avoidance response frequency (score) was measured by pressing a filament with a bending force of 0.16 g against the back of the hind paw. The results are shown in FIG. 22.

With reference to FIG. 22, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 0.2 mg/kg vincristine; the dotted line with white squares represents the group receiving 5 mg/kg D-threitol; and the broken line with black squares represents the group receiving 0.2 mg/kg vincristine+5 mg/kg D-threitol. A “*” mark was added to a measurement that was determined to have a significant difference from that of the control group in a test, using a significance level of 1% (in the figure, shown as “*P<0.01 vs control group”).

The group receiving 0.2 mg/kg vincristine (broken line with black circles) exhibited a remarkable increase in the avoidance response score compared with the control group (solid line with white circles). On the other hand, the group receiving 5 mg/kg D-threitol (dotted line with white squares), which received only D-threitol, and the group receiving 0.2 mg/kg vincristine+5 mg/kg D-threitol (broken line with black squares), which received D-threitol in combination, exhibited avoidance response scores similar to that of the control group (solid line with white circles) throughout the experimental period. In these two groups, an increase in the avoidance response score was suppressed compared with the group receiving 0.2 mg/kg vincristine (broken line with black circles).

Both the cold plate test (FIG. 21) and the von Frey test (FIG. 22) demonstrated that the group receiving 0.2 mg/kg vincristine (broken line with black circles) exhibited a significant change in both the avoidance response score and the withdrawal response time compared with the control group (solid line with white circles), the change being a shift to a greater degree of shunning the stimulus. This demonstrates that the administration of vincristine caused peripheral neuropathy (peripheral nerve hypersensitivity) to develop.

On the other hand, the group receiving 5 mg/kg D-threitol (dotted line with white squares) and the group receiving 0.2 mg/kg vincristine+5 mg/kg D-threitol (broken line with black squares), which received D-threitol, showed results similar to those of the control group. Therefore, it can be concluded that D-threitol suppresses the peripheral neuropathy (peripheral nerve hypersensitivity) induced by vincristine. In other words, D-threitol serves as a preventive composition (preventive agent) for peripheral neuropathy that is caused by vincristine (anticancer drug).

<Preventive Action of D-Threitol on Peripheral Neuropathy in a Mouse Caused by Bortezomib>

The preventive action of D-threitol on hyperesthesia that occurs when an anticancer drug, bortezomib, was administered was examined. Examples of hyperesthesia include paresthesia that is caused by a low temperature stimulus and allodynia (severe pain induced by a tactile stimulus that does not usually cause pain) that is caused by a mechanical stimulus. D-threitol was orally administered to a mouse simultaneously with the administration of bortezomib, and the following tests (cold plate test and von Frey test) were performed.

(10) Administration of Test Article

Six to seven-week-old female Balb/c mice were used for the tests as with Example 1. All the mice were acclimated for seven days after arrival. Then, the mice were grouped into the following four groups: a control group, a group receiving bortezomib, a group receiving D-threitol, and a group receiving bortezomib and D-threitol (a group receiving bortezomib+D-threitol). Each group consisted of five mice.

On Day 8 of acclimation, the mice in the group receiving bortezomib and the group receiving bortezomib+D-threitol were intraperitoneally administered with bortezomib at a dose of 1 mg/kg. This day was designated as Day 0 of administration. Thereafter, these mice were intraperitoneally administered with bortezomib at a daily dose of 1 mg/kg on Day 7 and Day 14.

The mice in the group receiving D-threitol and the group receiving bortezomib+D-threitol were orally administered with D-threitol at a dose of 5 mg/kg daily from Day 0.

These respective groups are referred to as the control group, the group receiving 1 mg/kg bortezomib, the group receiving 5 mg/kg D-threitol, and the group receiving 1 mg/kg bortezomib+5 mg/kg D-threitol.

(11) Cold Plate Test

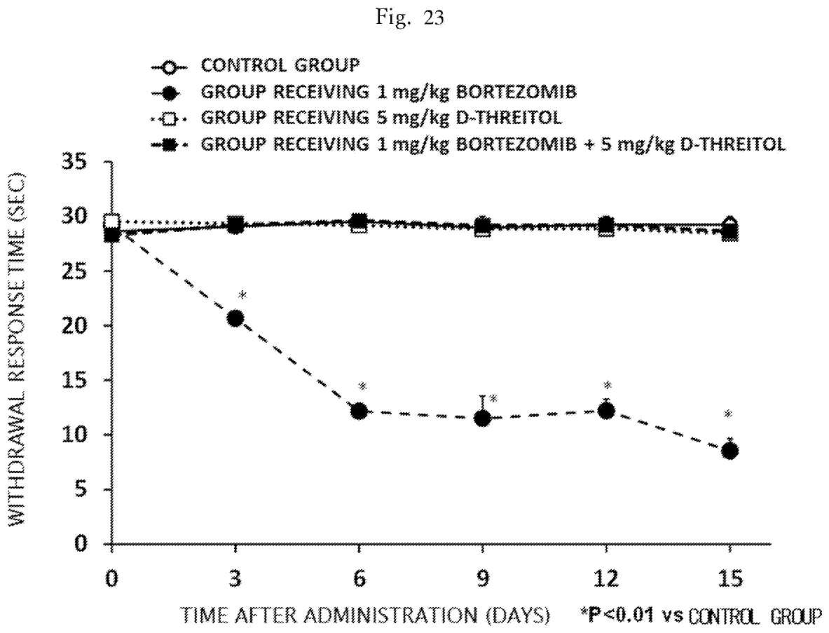

A cold plate test was performed to examine the effect of D-threitol on paresthesia that is caused by a low temperature stimulus. The mice in the four groups described in (10) of the present example (Example 3) were placed on a cold plate set at 10° C., and withdrawal response time (latent time) was measured. It is presumed that a shorter latent time reflects a greater degree of shunning the low temperature stimulus by the cold plate. The results are shown in FIG. 23.

With reference to FIG. 23, the horizontal axis represents the time after administration (days), and the vertical axis represents the average value of the withdrawal response time (seconds) of the mice in each group. The solid line with white circles represents the control group; the broken line with black circles represents the group receiving 1 mg/kg bortezomib; the dotted line with white squares represents the group receiving 5 mg/kg D-threitol; and the broken line with black squares represents the group receiving 1 mg/kg bortezomib+5 mg/kg D-threitol. A “*” mark was added to a measurement that was determined to have a significant difference from that of the control group in a test, using a significance level of 1% (in the figure, shown as “*P<0.01 vs control group”).

In the group receiving 1 mg/kg bortezomib (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg D-threitol (dotted line with white squares), which received only D-threitol, and the group receiving 1 mg/kg bortezomib+5 mg/kg D-threitol (broken line with black squares), which received bortezomib and D-threitol in combination, exhibited a withdrawal response time (latent time) approximately similar to that of the control group (solid line with white circles). The two groups receiving D-threitol also did not show a reduction in the withdrawal response time thereafter. In these two groups, the reduction in the withdrawal response time (latent time) was suppressed compared with the group receiving 1 mg/kg bortezomib (broken line with black circles).

(12) Von Frey Test

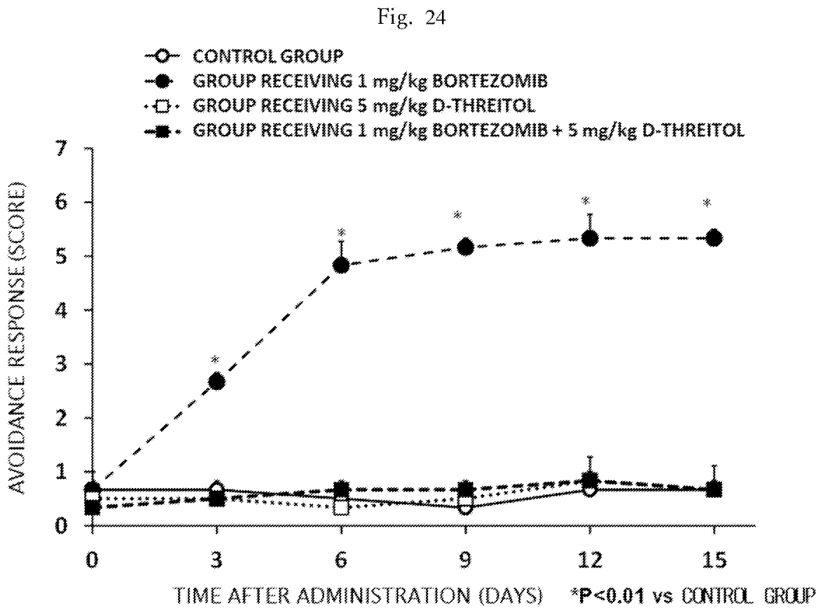

The mice in the four groups described in (10) of the present example (Example 3) were placed in a cage, and the avoidance response frequency (score) was measured by pressing a filament with a bending force of 0.16 g against the back of the hind paw. The results are shown in FIG. 24.

With reference to FIG. 24, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 1 mg/kg bortezomib; the dotted line with white squares represents the group receiving 5 mg/kg D-threitol; and the broken line with black squares represents the group receiving 1 mg/kg bortezomib+5 mg/kg D-threitol. A “*” mark was added to a measurement that was determined to have a significant difference from that of the control group in a test, using a significance level of 1% (in the figure, shown as “*P<0.01 vs control group”).

The group receiving 1 mg/kg bortezomib (broken line with black circles) exhibited a remarkable increase in the avoidance response score compared with the control group (solid line with white circles). On the other hand, the group receiving 5 mg/kg D-threitol (dotted line with white squares), which received only D-threitol, and the group receiving 1 mg/kg bortezomib+5 mg/kg D-threitol (broken line with black squares), which received D-threitol in combination, exhibited avoidance response scores similar to that of the control group (solid line with white circles) throughout the experimental period. In these two groups, an increase in the avoidance response score was suppressed compared with the group receiving 1 mg/kg bortezomib (broken line with black circles).

Both the cold plate test (FIG. 23) and the von Frey test (FIG. 24) demonstrated that the group receiving 1 mg/kg bortezomib (broken line with black circles) exhibited a significant change in both the withdrawal response time and the avoidance response score compared with the control group (solid line with white circles), the change being a shift to a greater degree of shunning the stimulus. This demonstrates that the administration of bortezomib caused peripheral neuropathy (peripheral nerve hypersensitivity) to develop.

On the other hand, the group receiving 5 mg/kg D-threitol (dotted line with white squares) and the group receiving 1 mg/kg bortezomib+5 mg/kg D-threitol (broken line with black squares), which received D-threitol, showed results similar to those of the control group. Therefore, it can be concluded that D-threitol suppresses the peripheral neuropathy (peripheral nerve hypersensitivity) induced by bortezomib. In other words, D-threitol serves as a preventive composition (preventive agent) for peripheral neuropathy that is caused by bortezomib (anticancer drug).

### (Example 4) D-Threitol-Containing Therapeutic Agent for Peripheral Neuropathy that is Caused by Administration of Anticancer Drug

### <Therapeutic Action of D-Threitol on Peripheral Neuropathy in a Mouse Caused by Oxaliplatin>

It was found that D-threitol is capable of preventing the peripheral neuropathy that is caused by oxaliplatin. Therefore, whether D-threitol had a therapeutic action of alleviating peripheral neuropathy after the development of peripheral neuropathy via the intake of an anticancer drug was subsequently examined.

(1) Administration of Test Article

Six to seven-week-old female Balb/c mice were used for the tests as with Example 1. All the mice were acclimated for seven days after arrival. Then, the mice were grouped into the following four groups: a control group, a group receiving oxaliplatin, a group receiving D-threitol, and a group receiving oxaliplatin and D-threitol (a group receiving oxaliplatin+D-threitol). Each group consisted of five mice.

On Day 8 of acclimation, the mice in the group receiving oxaliplatin and the group receiving oxaliplatin+D-threitol were intraperitoneally administered with oxaliplatin at a dose of 6 mg/kg. This day was designated as the first day of the administration (Day 0). Thereafter, these mice were intraperitoneally administered with oxaliplatin at the same dose on Day 7 and Day 14 for a total of 3 doses.

The mice in the group receiving 100 mg/kg D-threitol were orally administered with D-threitol at a dose of 100 mg/kg daily from Day 0. The mice in the group receiving oxaliplatin+100 mg/kg D-threitol were orally administered with D-threitol at a dose of 100 mg/kg daily from Day 6 of administration.

(2) Cold Plate Test

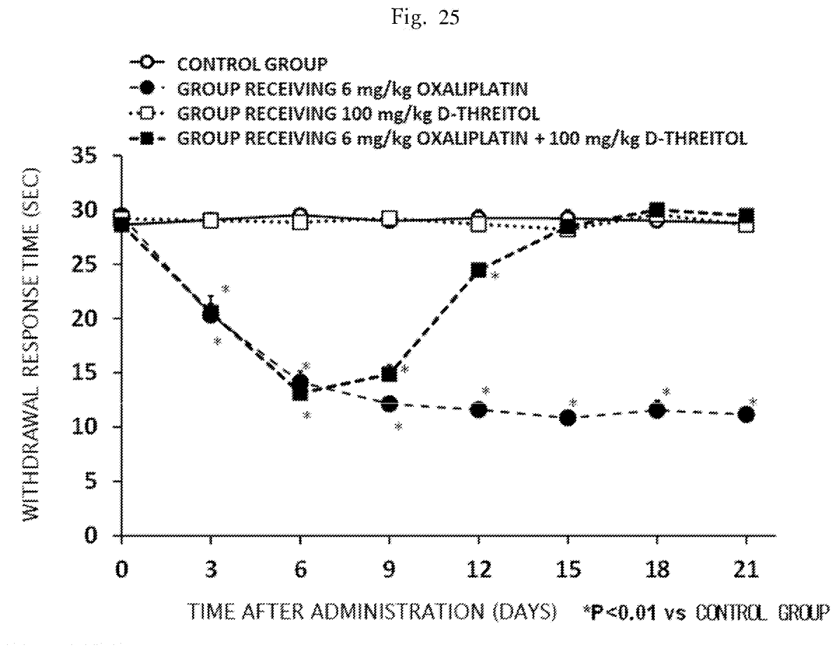

A test was performed on the mice in the four groups described in (1) of the present example (Example 4) to examine the effect of D-threitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 25.

With reference to FIG. 25, the horizontal axis represents the time after administration (days), and the vertical axis represents the average value of the withdrawal response time (seconds) of the mice in each group. The solid line with white circles represents the control group; the broken line with black circles represents the group receiving 6 mg/kg oxaliplatin; the dotted line with white squares represents the group receiving 100 mg/kg D-threitol; and the broken line with black squares represents the group receiving 6 mg/kg oxaliplatin+100 mg/kg D-threitol. A “*” mark was added to a measurement that was determined to have a significant difference from that of the control group in a test, using a significance level of 1% (in the figure, shown as “*P<0.01 vs control group”).

With reference to FIG. 25, in the groups receiving oxaliplatin other than the control group and the group receiving D-threitol, a uniform reduction in the withdrawal response time (latent time) was observed by Day 6 after administration. However, the latent time tended to become longer from Day 6 onwards in the group receiving 6 mg/kg oxaliplatin+100 mg/kg D-threitol. On Day 15, the group receiving 6 mg/kg oxaliplatin+100 mg/kg D-threitol had a withdrawal response time similar to that of the control group (solid line with white circles).

The withdrawal response time (latent time) was greatly reduced on Day 6 of administration in the two groups receiving oxaliplatin (the group receiving 6 mg/kg oxaliplatin, and the group receiving 6 mg/kg oxaliplatin+100 mg/kg D-threitol). Thus, it is presumed that the mice in these groups developed peripheral neuropathy (peripheral nerve hypersensitivity).

After that, the group receiving D-threitol exhibited a significantly longer withdrawal response time (latent time) compared with the group receiving 6 mg/kg oxaliplatin and no D-threitol. This result signifies that the peripheral neuropathy (peripheral nerve hypersensitivity) that had developed beforehand was ameliorated by taking D-threitol. Thus, it was found that D-threitol also serves as a therapeutic composition (therapeutic agent) for peripheral neuropathy (peripheral nerve hypersensitivity).

(3) Von Frey Test

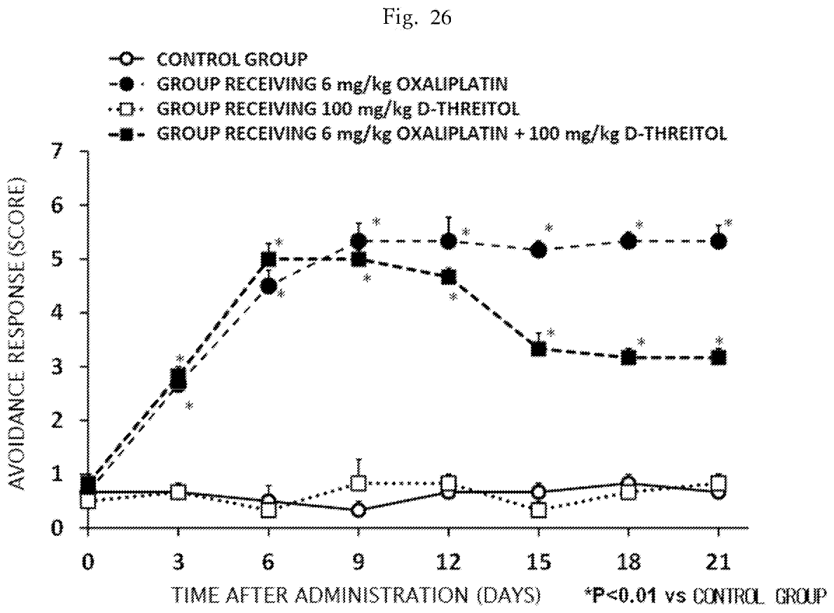

For the mice in the four groups described in (1) of the present example (Example 4), the avoidance response frequency (score) was measured by pressing a filament with a bending force of 0.16 g against the back of the hind paw. The results are shown in FIG. 26.

With reference to FIG. 26, the horizontal axis represents the time after administration (days), and the vertical axis represents the average value of the avoidance responses (score) of the mice in each group. The solid line with white circles represents the control group; the broken line with black circles represents the group receiving 6 mg/kg oxaliplatin; the dotted line with white squares represents the group receiving 100 mg/kg D-threitol; and the broken line with black squares represents the group receiving 6 mg/kg oxaliplatin+100 mg/kg D-threitol. A “*” mark was added to a measurement that was determined to have a significant difference from that of the control group in a test, using a significance level of 1% (in the figure, shown as “*P<0.01 vs control group”).

The group receiving oxaliplatin (broken line with black circles) exhibited a remarkable increase in the avoidance response score compared with the control group (solid line with white circles). Thus, it is presumed that the mice in the group receiving oxaliplatin developed peripheral neuropathy (peripheral nerve hypersensitivity). On the other hand, the group receiving 6 mg/kg oxaliplatin+100 mg/kg D-threitol (broken line with black squares), which received D-threitol in combination, maintained a score clearly lower than that of the group receiving oxaliplatin (broken line with black circles) from Day 15 onwards. Thus, it was found that D-threitol also serves as a therapeutic composition (therapeutic agent) for peripheral neuropathy (peripheral nerve hypersensitivity).

<Therapeutic Action of D-Threitol on Peripheral Neuropathy in a Mouse Caused by Paclitaxel>

It was found that D-threitol is capable of preventing the peripheral neuropathy that is caused by paclitaxel. Therefore, whether D-threitol had a therapeutic action of alleviating peripheral neuropathy after the development of peripheral neuropathy via the intake of an anticancer drug was subsequently examined.

(4) Administration of Test Article

Six to seven-week-old female Balb/c mice were used for the tests as with Example 1. All the mice were acclimated for seven days after arrival. Then, the mice were grouped into the following four groups: a control group, a group receiving paclitaxel, a group receiving D-threitol, and a group receiving paclitaxel and D-threitol (a group receiving paclitaxel+D-threitol). Each group consisted of five mice.

On Day 8 of acclimation, the mice in the group receiving paclitaxel and the group receiving paclitaxel+D-threitol were intraperitoneally administered with paclitaxel at a dose of 6 mg/kg. This day was designated as the first day of the administration (Day 0). Thereafter, these mice were intraperitoneally administered with paclitaxel at the same dose on Day 7 and Day 14 for a total of 3 doses.

The mice in the group receiving 100 mg/kg D-threitol were orally administered with D-threitol at a dose of 100 mg/kg daily from Day 0. The mice in the group receiving paclitaxel+100 mg/kg D-threitol were orally administered with D-threitol at a dose of 100 mg/kg daily from Day 6 of administration.

(5) Cold Plate Test

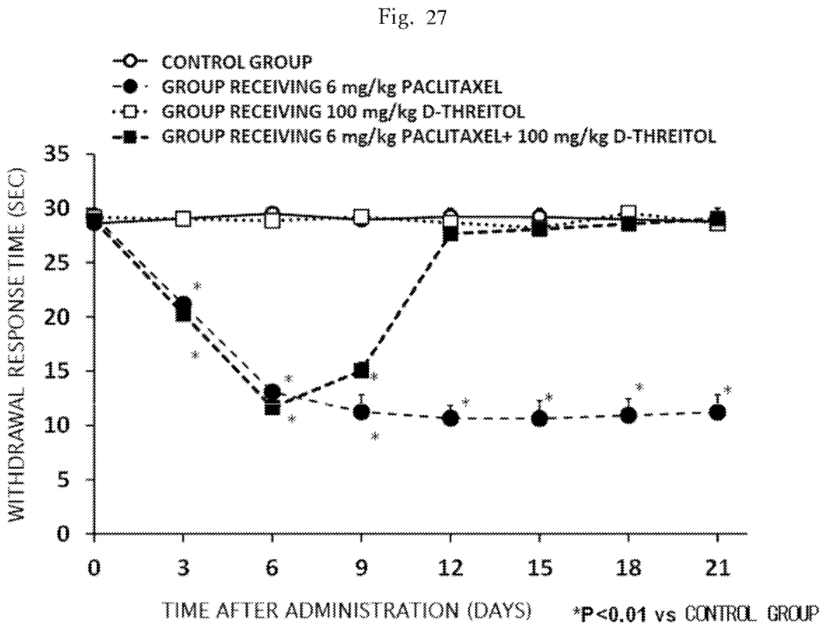

A test was performed on the mice in the four groups described in (4) of the present example (Example 4) to examine the effect of D-threitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 27.

With reference to FIG. 27, the horizontal axis represents the time after administration (days), and the vertical axis represents the average value of the withdrawal response time (seconds) of the mice in each group. The solid line with white circles represents the control group; the broken line with black circles represents the group receiving 6 mg/kg paclitaxel; the dotted line with white squares represents the group receiving 100 mg/kg D-threitol; and the broken line with black squares represents the group receiving 6 mg/kg paclitaxel+100 mg/kg D-threitol. A “*” mark was added to a measurement that was determined to have a significant difference from that of the control group in a test, using a significance level of 1% (in the figure, shown as “*P<0.01 vs control group”).

With reference to FIG. 27, in the two groups receiving paclitaxel other than the control group and the group receiving D-threitol, a uniform reduction in the withdrawal response time (latent time) was observed by Day 6 after administration. However, the latent time tended to become longer from Day 6 onwards in the group receiving 6 mg/kg paclitaxel+100 mg/kg D-threitol. On Day 12, the group receiving 6 mg/kg paclitaxel+100 mg/kg D-threitol had a significantly longer withdrawal response time (latent time) compared with the group receiving 6 mg/kg paclitaxel.

The withdrawal response time (latent time) was greatly reduced on Day 6 of administration in the two groups receiving paclitaxel (the group receiving 6 mg/kg paclitaxel, and the group receiving 6 mg/kg paclitaxel+100 mg/kg D-threitol). Thus, it is presumed that the mice in these groups developed peripheral neuropathy (peripheral nerve hypersensitivity).

After that, the groups receiving D-threitol exhibited a significantly longer withdrawal response time (latent time) compared with the group receiving 6 mg/kg paclitaxel and no D-threitol. This result signifies that the peripheral neuropathy (peripheral nerve hypersensitivity) that had developed beforehand was ameliorated by taking D-threitol. Thus, it was found that D-threitol also serves as a therapeutic composition (therapeutic agent) for peripheral neuropathy (peripheral nerve hypersensitivity).

(6) Von Frey Test

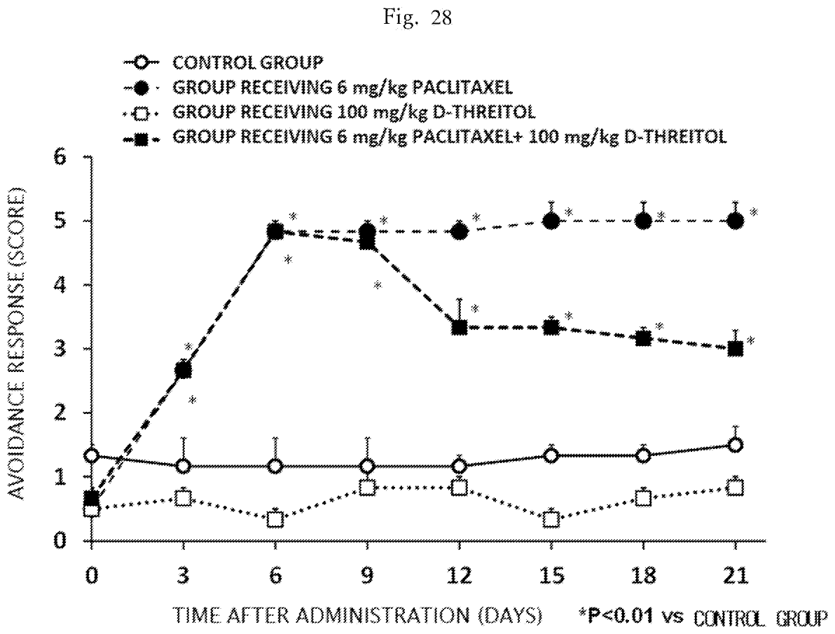

For the mice in the four groups described in (4) of the present example (Example 4), the avoidance response frequency (score) was measured by pressing a filament with a bending force of 0.16 g against the back of the hind paw. The results are shown in FIG. 28.

With reference to FIG. 28, the horizontal axis represents the time after administration (days), and the vertical axis represents the average value of the avoidance responses (score) of the mice in each group. The solid line with white circles represents the control group; the broken line with black circles represents the group receiving 6 mg/kg paclitaxel; the dotted line with white squares represents the group receiving 100 mg/kg D-threitol; and the broken line with black squares represents the group receiving 6 mg/kg paclitaxel+100 mg/kg D-threitol. A “*” mark was added to a measurement that was determined to have a significant difference from that of the control group in a test, using a significance level of 1% (in the figure, shown as “*P<0.01 vs control group”).

The group receiving paclitaxel (broken line with black circles) exhibited a remarkable increase in the avoidance response score compared with the control group (solid line with white circles). Thus, it is presumed that the mice in the group receiving paclitaxel developed peripheral neuropathy (peripheral nerve hypersensitivity). On the other hand, the group receiving 6 mg/kg paclitaxel+100 mg/kg D-threitol (broken line with black squares), which received D-threitol in combination, maintained a score clearly lower than that of the group receiving paclitaxel (broken line with black circles) from Day 12 onwards. Thus, it was found that D-threitol also serves as a therapeutic composition (therapeutic agent) for peripheral neuropathy (peripheral nerve hypersensitivity).

<Therapeutic Action of D-Threitol on Peripheral Neuropathy in a Mouse Caused by Vincristine>

It was found that D-threitol is capable of preventing the peripheral neuropathy that is caused by vincristine. Therefore, whether D-threitol had a therapeutic action of alleviating peripheral neuropathy after the development of peripheral neuropathy via the intake of an anticancer drug was subsequently examined.

(7) Administration of Test Article

Six to seven-week-old female Balb/c mice were used for the tests as with Example 1. All the mice were acclimated for seven days after arrival. Then, the mice were grouped into the following four groups: a control group, a group receiving vincristine, a group receiving D-threitol, and a group receiving vincristine and D-threitol (a group receiving vincristine+D-threitol). Each group consisted of five mice.

On Day 8 of acclimation, the mice in the group receiving vincristine and the group receiving vincristine+D-threitol were intraperitoneally administered with vincristine at a dose of 0.2 mg/kg. This day was designated as the first day of the administration (Day 0). Thereafter, these mice were intraperitoneally administered with vincristine at the same dose on Day 7 and Day 14 for a total of 3 doses.

The mice in the group receiving 100 mg/kg D-threitol were orally administered with D-threitol at a dose of 100 mg/kg daily from Day 0. The mice in the group receiving vincristine+100 mg/kg D-threitol were orally administered with D-threitol at a dose of 100 mg/kg daily from Day 6 of administration.

(8) Cold Plate Test

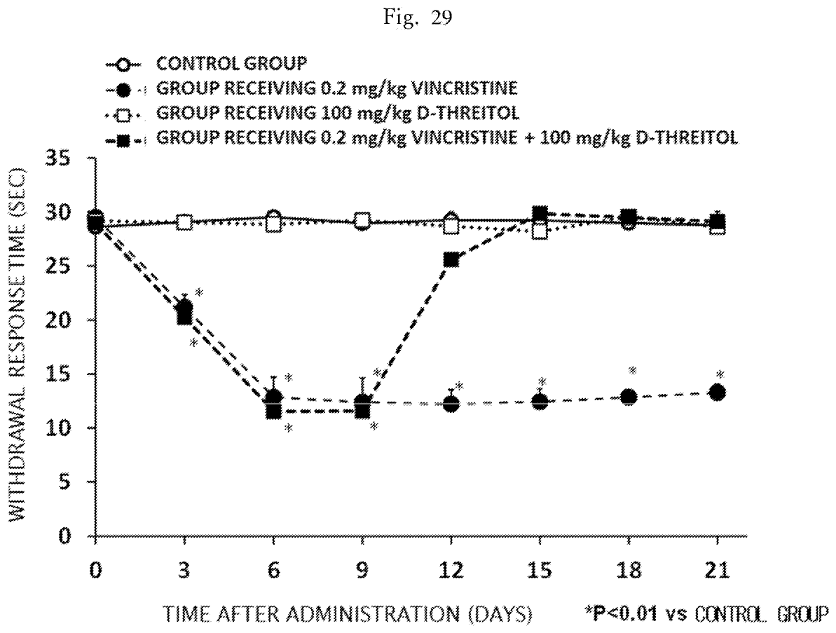

A test was performed on the mice in the four groups described in (7) of the present example (Example 4) to examine the effect of D-threitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 29.

With reference to FIG. 29, the horizontal axis represents the time after administration (days), and the vertical axis represents the average value of the withdrawal response time (seconds) of the mice in each group. The solid line with white circles represents the control group; the broken line with black circles represents the group receiving 0.2 mg/kg vincristine; the dotted line with white squares represents the group receiving 100 mg/kg D-threitol; and the broken line with black squares represents the group receiving 0.2 mg/kg vincristine+100 mg/kg D-threitol. A “*” mark was added to a measurement that was determined to have a significant difference from that of the control group in a test, using a significance level of 1% (in the figure, shown as “*P<0.01 vs control group”).

With reference to FIG. 29, in the two groups receiving vincristine other than the control group and the group receiving D-threitol, a uniform reduction in the withdrawal response time (latent time) was observed by Day 6 after administration. However, on Day 12, the group receiving 0.2 mg/kg vincristine+100 mg/kg D-threitol had a significantly longer withdrawal response time (latent time) compared with the group receiving 0.2 mg/kg vincristine.

The withdrawal response time (latent time) was greatly reduced on Day 6 of administration in the two groups receiving vincristine (the group receiving 0.2 mg/kg vincristine, and the group receiving 0.2 mg/kg vincristine+100 mg/kg D-threitol). Thus, it is presumed that the mice in these groups developed peripheral neuropathy (peripheral nerve hypersensitivity).

After that, the groups receiving D-threitol exhibited a significantly longer withdrawal response time (latent time) compared with the group receiving 0.2 mg/kg vincristine and no D-threitol. This result signifies that the peripheral neuropathy (peripheral nerve hypersensitivity) that had developed beforehand was ameliorated by taking D-threitol. Thus, it was found that D-threitol also serves as a therapeutic composition (therapeutic agent) for peripheral neuropathy (peripheral nerve hypersensitivity).

(9) Von Frey Test

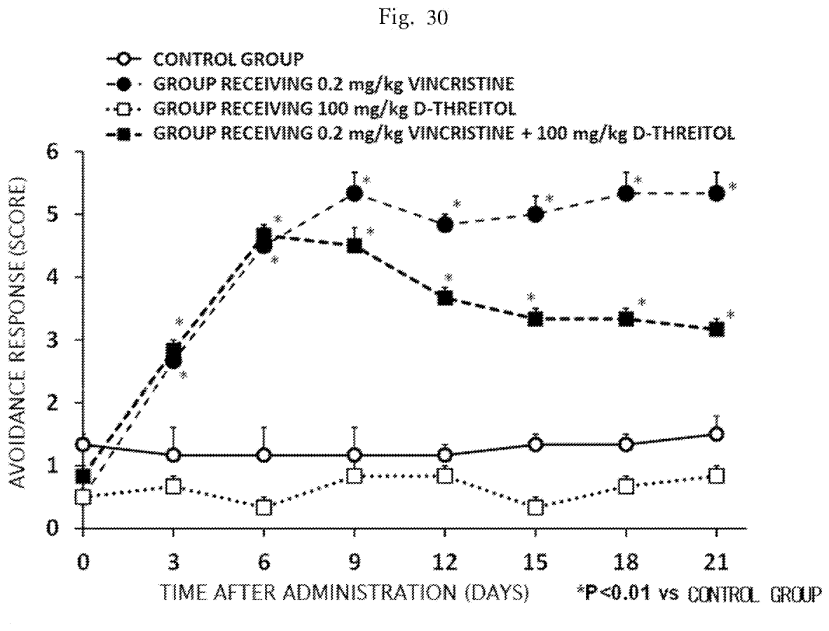

For the mice in the four groups described in (7) of the present example (Example 4), the avoidance response frequency (score) was measured by pressing a filament with a bending force of 0.16 g against the back of the hind paw. The results are shown in FIG. 30.

With reference to FIG. 30, the horizontal axis represents the time after administration (days), and the vertical axis represents the average value of the avoidance responses (score) of the mice in each group. The solid line with white circles represents the control group; the broken line with black circles represents the group receiving 0.2 mg/kg vincristine; the dotted line with white squares represents the group receiving 100 mg/kg D-threitol; and the broken line with black squares represents the group receiving 0.2 mg/kg vincristine+100 mg/kg D-threitol. A “*” mark was added to a measurement that was determined to have a significant difference from that of the control group in a test, using a significance level of 1% (in the figure, shown as “*P<0.01 vs control group”).

The group receiving vincristine (broken line with black circles) exhibited a remarkable increase in the avoidance response score compared with the control group (solid line with white circles). Thus, it is presumed that the mice in the group receiving vincristine developed peripheral neuropathy (peripheral nerve hypersensitivity) to develop. On the other hand, the group receiving 0.2 mg/kg vincristine+100 mg/kg D-threitol (broken line with black squares), which received D-threitol in combination, maintained a score clearly lower than that of the group receiving vincristine (broken line with black circles) from Day 12 onwards. Thus, it was found that D-threitol also serves as a therapeutic composition (therapeutic agent) for peripheral neuropathy (peripheral nerve hypersensitivity).

<Therapeutic Action of D-Threitol on Peripheral Neuropathy in a Mouse Caused by Bortezomib>

It was found that D-threitol is capable of preventing the peripheral neuropathy that is caused by bortezomib. Therefore, whether D-threitol had a therapeutic action of alleviating peripheral neuropathy after the development of peripheral neuropathy via the intake of an anticancer drug was subsequently examined.

(10) Administration of Test Article

Six to seven-week-old female Balb/c mice were used for the tests as with Example 1. All the mice were acclimated for seven days after arrival. Then, the mice were grouped into the following four groups: a control group, a group receiving bortezomib, a group receiving D-threitol, and a group receiving bortezomib and D-threitol (a group receiving bortezomib+D-threitol). Each group consisted of five mice.

On Day 8 of acclimation, the mice in the group receiving bortezomib and the group receiving bortezomib+D-threitol were intraperitoneally administered with bortezomib at a dose of 1 mg/kg. This day was designated as the first day of the administration (Day 0). Thereafter, these mice were intraperitoneally administered with bortezomib at the same dose on Day 7 and Day 14 for a total of 3 doses.

The mice in the group receiving 100 mg/kg D-threitol were orally administered with D-threitol at a dose of 100 mg/kg daily from Day 0. The mice in the group receiving bortezomib+100 mg/kg D-threitol were orally administered with D-threitol at a dose of 100 mg/kg daily from Day 6 of administration.

(11) Cold Plate Test

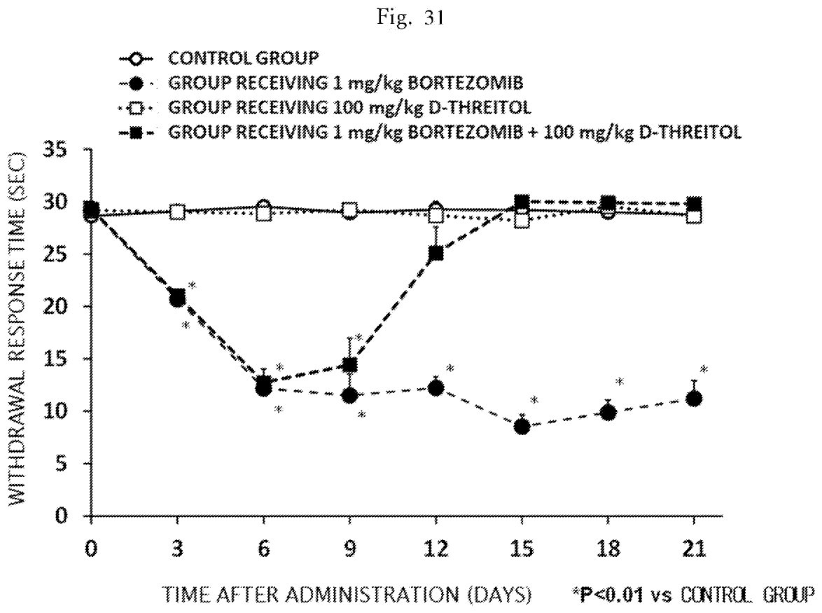

A test was performed on the mice in the four groups described in (10) of the present example (Example 4) to examine the effect of D-threitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 31.

With reference to FIG. 31, the horizontal axis represents the time after administration (days), and the vertical axis represents the average value of the withdrawal response time (seconds) of the mice in each group. The solid line with white circles represents the control group; the broken line with black circles represents the group receiving 1 mg/kg bortezomib; the dotted line with white squares represents the group receiving 100 mg/kg D-threitol; and the broken line with black squares represents the group receiving 1 mg/kg bortezomib+100 mg/kg D-threitol. A “*” mark was added to a measurement that was determined to have a significant difference from that of the control group in a test, using a significance level of 1% (in the figure, shown as “*P<0.01 vs control group”).

With reference to FIG. 31, in the two groups receiving bortezomib other than the control group and the group receiving D-threitol, a uniform reduction in the withdrawal response time (latent time) was observed by Day 6 after administration. However, the latent time tended to become longer from Day 6 onwards in the group receiving 1 mg/kg bortezomib+100 mg/kg D-threitol. On Day 12, the group receiving 1 mg/kg bortezomib+100 mg/kg D-threitol had a significantly longer withdrawal response time (latent time) compared with the group receiving 1 mg/kg bortezomib.

The withdrawal response time (latent time) was greatly reduced on Day 6 of administration in the two groups receiving bortezomib (the group receiving 1 mg/kg bortezomib, and the group receiving 1 mg/kg bortezomib+100 mg/kg D-threitol). Thus, it is presumed that the mice in these groups developed peripheral neuropathy (peripheral nerve hypersensitivity).

After that, the groups receiving D-threitol exhibited a significantly longer withdrawal response time (latent time) compared with the group receiving 1 mg/kg bortezomib and no D-threitol. This result signifies that the peripheral neuropathy (peripheral nerve hypersensitivity) that had developed beforehand was ameliorated by taking D-threitol. Thus, it was found that D-threitol also serves as a therapeutic composition (therapeutic agent) for peripheral neuropathy (peripheral nerve hypersensitivity).

(12) Von Frey Test

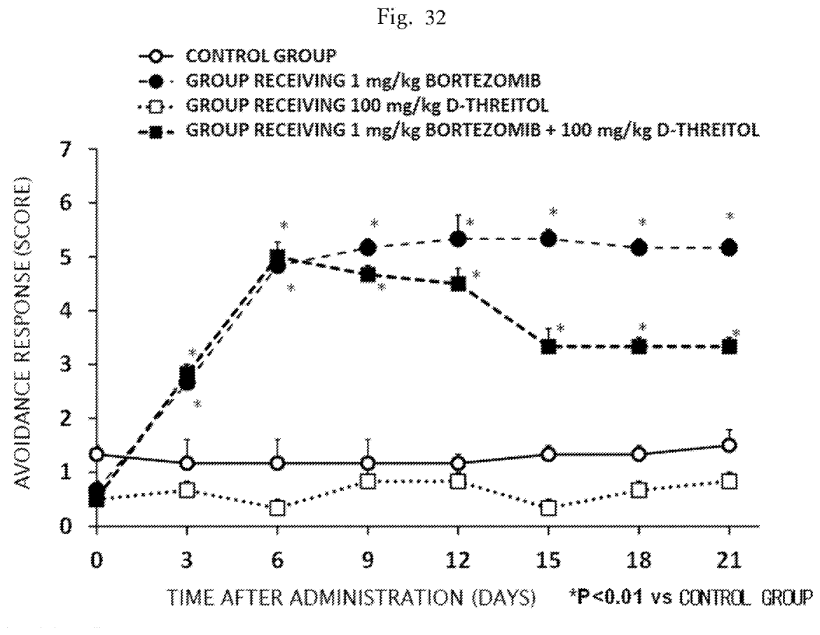

For the mice in the four groups described in (10) of the present example (Example 4), the avoidance response frequency (score) was measured by pressing a filament with a bending force of 0.16 g against the back of the hind paw. The results are shown in FIG. 32.

With reference to FIG. 32, the horizontal axis represents the time after administration (days), and the vertical axis represents the average value of the avoidance responses (score) of the mice in each group. The solid line with white circles represents the control group; the broken line with black circles represents the group receiving 1 mg/kg bortezomib; the dotted line with white squares represents the group receiving 100 mg/kg D-threitol; and the broken line with black squares represents the group receiving 1 mg/kg bortezomib+100 mg/kg D-threitol. A “*” mark was added to a measurement that was determined to have a significant difference from that of the control group in a test, using a significance level of 1% (in the figure, shown as “*P<0.01 vs control group”).

The group receiving bortezomib (broken line with black circles) exhibited a remarkable increase in the avoidance response score compared with the control group (solid line with white circles). Thus, it is presumed that the mice in the group receiving bortezomib developed peripheral neuropathy (peripheral nerve hypersensitivity). On the other hand, the group receiving 1 mg/kg bortezomib+100 mg/kg D-threitol (broken line with black squares), which received D-threitol in combination, maintained a score clearly lower than that of the group receiving bortezomib (broken line with black circles) from Day 12 onwards. Thus, it was found that D-threitol also serves as a therapeutic composition (therapeutic agent) for peripheral neuropathy (peripheral nerve hypersensitivity).

### (Example 5) L-Talitol-Containing Preventive Agent for Peripheral Neuropathy that is Caused by Administration of Anticancer Drug

### <Preventive Action of L-Talitol on Peripheral Neuropathy in a Mouse Caused by Oxaliplatin>

The preventive action of L-talitol on hyperesthesia that occurs when an anticancer drug, oxaliplatin, was administered was examined. Examples of hyperesthesia include paresthesia that is caused by a low temperature stimulus and allodynia (severe pain induced by a tactile stimulus that does not usually cause pain) that is caused by a mechanical stimulus. L-talitol was orally administered to a mouse simultaneously with the administration of oxaliplatin, and the following tests (cold plate test and von Frey test) were performed.

(1) Administration of Test Article

Six to seven-week-old female Balb/c mice were used for the tests as with Example 1. All the mice were acclimated for seven days after arrival. Then, the mice were grouped into the following four groups: a control group, a group receiving oxaliplatin, a group receiving L-talitol, and a group receiving oxaliplatin and L-talitol (a group receiving oxaliplatin+L-talitol). Each group consisted of five mice.

On Day 8 of acclimation, the mice in the group receiving oxaliplatin and the group receiving oxaliplatin+L-talitol were intraperitoneally administered with oxaliplatin at a dose of 6 mg/kg. This day was designated as the first day of the administration (Day 0). Thereafter, these mice were intraperitoneally administered with oxaliplatin at a daily dose of 6 mg/kg on Day 7 and Day 14.

The mice in the group receiving L-talitol and the group receiving oxaliplatin+L-talitol were orally administered with L-talitol at a dose of 5 mg/kg daily from Day 0.

These respective groups are referred to as the control group, the group receiving 6 mg/kg oxaliplatin, the group receiving 5 mg/kg L-talitol, and the group receiving 6 mg/kg oxaliplatin+5 mg/kg L-talitol.

(2) Cold Plate Test

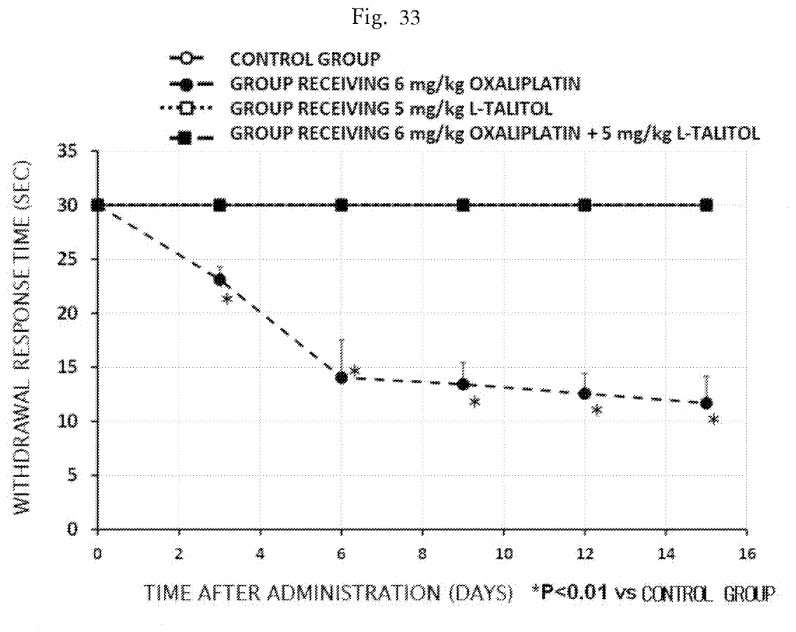

A cold plate test was performed to examine the effect of L-talitol on paresthesia that is caused by a low temperature stimulus. The mice in the four groups described in (1) of the present example (Example 5) were placed on a cold plate set at 10° C., and withdrawal response time (latent time) was measured. It is presumed that a shorter withdrawal response time (latent time) reflects a greater degree of shunning the low temperature stimulus by the cold plate. The results are shown in FIG. 33.

With reference to FIG. 33, the horizontal axis represents the time after administration (days), and the vertical axis represents the average value of the withdrawal response time (seconds) of the mice in each group. The solid line with white circles represents the control group; the broken line with black circles represents the group receiving 6 mg/kg oxaliplatin; the dotted line with white squares represents the group receiving 5 mg/kg L-talitol; and the broken line with black squares represents the group receiving 6 mg/kg oxaliplatin+5 mg/kg L-talitol. A “*” mark was added to a measurement that was determined to have a significant difference from that of the control group in a test, using a significance level of 1% (in the figure, shown as “*P<0.01 vs control group”).

The control group, the group receiving 5 mg/kg L-talitol, and the group receiving 6 mg/kg oxaliplatin+5 mg/kg L-talitol had almost the same data, and the three lines overlapped in FIG. 33, making it difficult to see the data of the control group and the group receiving 5 mg/kg L-talitol.

In the group receiving 6 mg/kg oxaliplatin (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg L-talitol (dotted line with white squares), which received only L-talitol, and the group receiving 6 mg/kg oxaliplatin+5 mg/kg L-talitol (broken line with black squares), which received oxaliplatin and L-talitol in combination, exhibited a withdrawal response time (latent time) approximately similar to that of the control group (solid line with white circles). The two groups receiving L-talitol also did not show a reduction in the withdrawal response time thereafter. In these two groups, the reduction in the withdrawal response time (latent time) was suppressed compared with the group receiving 6 mg/kg oxaliplatin (broken line with black circles).

(3) Von Frey Test

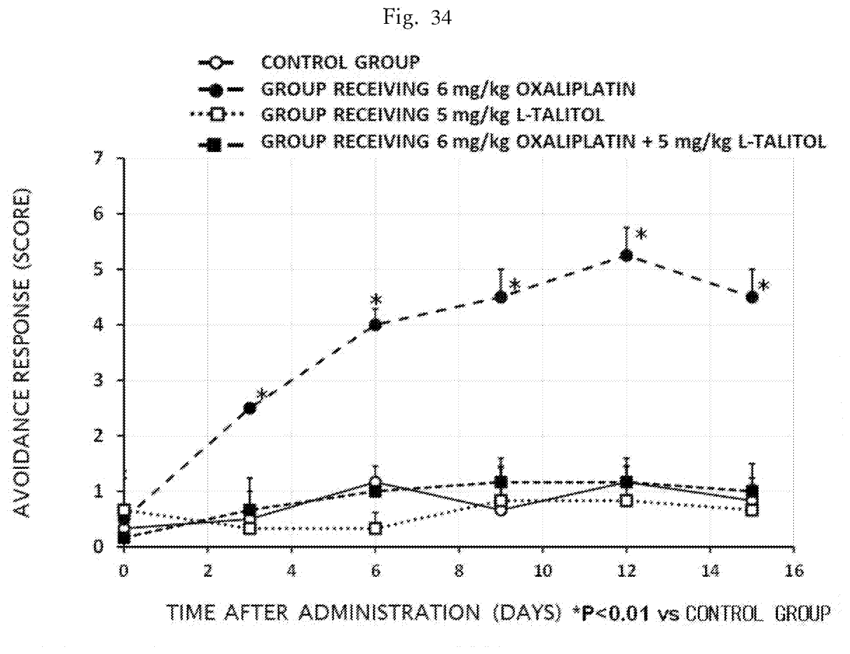

The mice in the four groups described in (1) of the present example (Example 5) were placed in a cage, and the avoidance response frequency (score) was measured by pressing a filament with a bending force of 0.16 g against the back of the hind paw. The results are shown in FIG. 34.

With reference to FIG. 34, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 6 mg/kg oxaliplatin; the dotted line with white squares represents the group receiving 5 mg/kg L-talitol; and the broken line with black squares represents the group receiving 6 mg/kg oxaliplatin+5 mg/kg L-talitol. A “*” mark was added to a measurement that was determined to have a significant difference from that of the control group in a test, using a significance level of 1% (in the figure, shown as “*P<0.01 vs control group”).

The group receiving 6 mg/kg oxaliplatin (broken line with black circles) exhibited a remarkable increase in the avoidance response score compared with the control group (solid line with white circles). On the other hand, the group receiving 5 mg/kg L-talitol (dotted line with white squares), which received only L-talitol, and the group receiving 6 mg/kg oxaliplatin+5 mg/kg L-talitol (broken line with black squares), which received L-talitol in combination, exhibited avoidance response scores similar to that of the control group (solid line with white circles) throughout the experimental period. In these two groups, an increase in the avoidance response score was suppressed compared with the group receiving 6 mg/kg oxaliplatin (broken line with black circles).

Both the cold plate test (FIG. 33) and the von Frey test (FIG. 34) demonstrated that the group receiving 6 mg/kg oxaliplatin (broken line with black circles) exhibited a significant change in both the withdrawal response time and the avoidance response score compared with the control group (solid line with white circles), the change being a shift to a greater degree of shunning the stimulus. This demonstrates that the administration of oxaliplatin caused peripheral neuropathy (peripheral nerve hypersensitivity) to develop.

On the other hand, the group receiving 5 mg/kg L-talitol (dotted line with white squares) and the group receiving 6 mg/kg oxaliplatin+5 mg/kg L-talitol (broken line with black squares), which received L-talitol, showed results similar to those of the control group. Therefore, it can be concluded that L-talitol suppresses the peripheral neuropathy (peripheral nerve hypersensitivity) induced by oxaliplatin. In other words, L-talitol serves as a preventive composition (preventive agent) for peripheral neuropathy that is caused by oxaliplatin (anticancer drug).

<Preventive Action of L-Talitol on Peripheral Neuropathy in a Mouse Caused by Paclitaxel>

The preventive action of L-talitol on hyperesthesia that occurs when an anticancer drug, paclitaxel, was administered was examined. Examples of hyperesthesia include paresthesia that is caused by a low temperature stimulus and allodynia (severe pain induced by a tactile stimulus that does not usually cause pain) that is caused by a mechanical stimulus. L-talitol was orally administered to a mouse simultaneously with the administration of paclitaxel, and the following tests (cold plate test and von Frey test) were performed.

(4) Administration of Test Article

Six to seven-week-old female Balb/c mice were used for the tests as with Example 1. All the mice were acclimated for seven days after arrival. Then, the mice were grouped into the following four groups: a control group, a group receiving paclitaxel, a group receiving L-talitol, and a group receiving paclitaxel and L-talitol (a group receiving paclitaxel+L-talitol). Each group consisted of five mice.

On Day 8 of acclimation, the mice in the group receiving paclitaxel and the group receiving paclitaxel+L-talitol were intraperitoneally administered with paclitaxel at a dose of 6 mg/kg. This day was designated as Day 0 of administration. Thereafter, these mice were intraperitoneally administered with paclitaxel at a daily dose of 6 mg/kg on Day 7 and Day 14.

The mice in the group receiving L-talitol and the group receiving paclitaxel+L-talitol were orally administered with L-talitol at a dose of 5 mg/kg daily from Day 0.

These respective groups are referred to as the control group, the group receiving 6 mg/kg paclitaxel, the group receiving 5 mg/kg L-talitol, and the group receiving 6 mg/kg paclitaxel+5 mg/kg L-talitol.

(5) Cold Plate Test

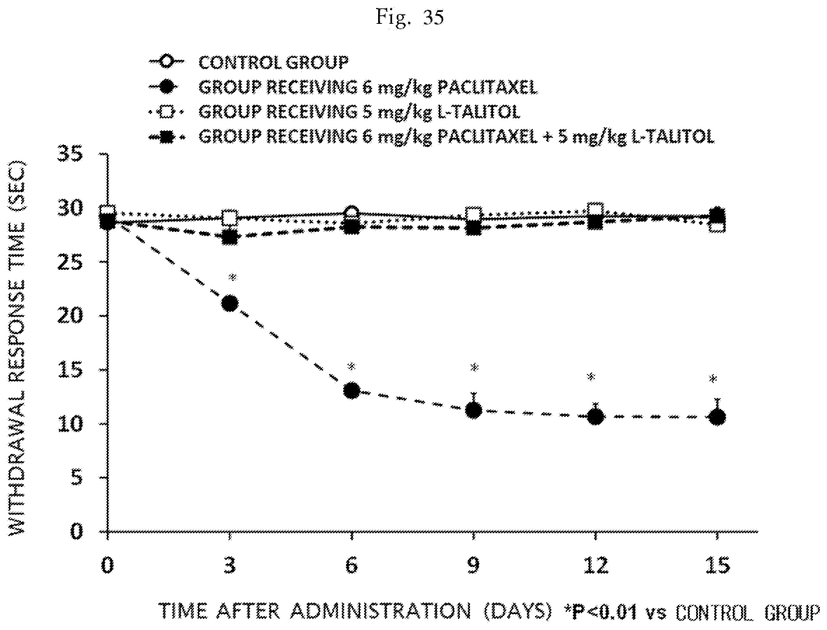

A cold plate test was performed to examine the effect of L-talitol on paresthesia that is caused by a low temperature stimulus. The mice in the four groups described in (4) of the present example (Example 5) were placed on a cold plate set at 10° C., and withdrawal response time (latent time) was measured. It is presumed that a shorter latent time reflects a greater degree of shunning the low temperature stimulus by the cold plate. The results are shown in FIG. 35.

With reference to FIG. 35, the horizontal axis represents the time after administration (days), and the vertical axis represents the average value of the withdrawal response time (seconds) of the mice in each group. The solid line with white circles represents the control group; the broken line with black circles represents the group receiving 6 mg/kg paclitaxel; the dotted line with white squares represents the group receiving 5 mg/kg L-talitol; and the broken line with black squares represents the group receiving 6 mg/kg paclitaxel+5 mg/kg L-talitol. A “*” mark was added to a measurement that was determined to have a significant difference from that of the control group in a test, using a significance level of 1% (in the figure, shown as “*P<0.01 vs control group”).

In the group receiving 6 mg/kg paclitaxel (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg L-talitol (dotted line with white squares), which received only L-talitol, and the group receiving 6 mg/kg paclitaxel+5 mg/kg L-talitol (broken line with black squares), which received paclitaxel and L-talitol in combination, exhibited a withdrawal response time (latent time) approximately similar to that of the control group (solid line with white circles). The two groups receiving L-talitol also did not show a reduction in the withdrawal response time thereafter. In these two groups, the reduction in the withdrawal response time (latent time) was suppressed compared with the group receiving 6 mg/kg paclitaxel (broken line with black circles).

(6) Von Frey Test

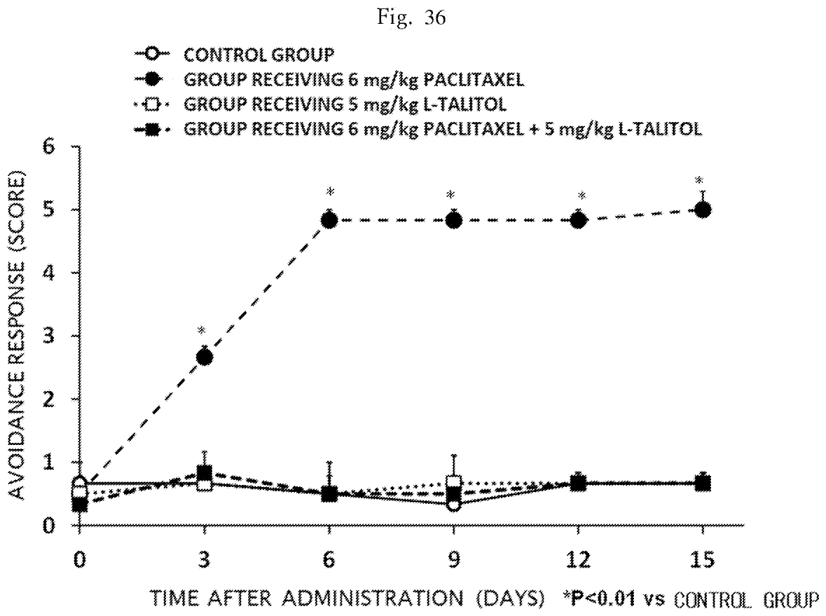

The mice in the four groups described in (4) of the present example (Example 5) were placed in a cage, and the avoidance response frequency (score) was measured by pressing a filament with a bending force of 0.16 g against the back of the hind paw. The results are shown in FIG. 36.

With reference to FIG. 36, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 6 mg/kg paclitaxel; the dotted line with white squares represents the group receiving 5 mg/kg L-talitol; and the broken line with black squares represents the group receiving 6 mg/kg paclitaxel+5 mg/kg L-talitol. A “*” mark was added to a measurement that was determined to have a significant difference from that of the control group in a test, using a significance level of 1% (in the figure, shown as “*P<0.01 vs control group”).

The group receiving 6 mg/kg paclitaxel (broken line with black circles) exhibited a remarkable increase in the avoidance response score compared with the control group (solid line with white circles). On the other hand, the group receiving 5 mg/kg L-talitol (dotted line with white squares), which received only L-talitol, and the group receiving 6 mg/kg paclitaxel+5 mg/kg L-talitol (broken line with black squares), which received L-talitol in combination, exhibited avoidance response scores similar to that of the control group (solid line with white circles) throughout the experimental period. In these two groups, an increase in the avoidance response score was suppressed compared with the group receiving 6 mg/kg paclitaxel (broken line with black circles).

Both the cold plate test (FIG. 35) and the von Frey test (FIG. 36) demonstrated that the group receiving 6 mg/kg paclitaxel (broken line with black circles) exhibited a significant change in both the withdrawal response time and the avoidance response score compared with the control group (solid line with white circles), the change being a shift to a greater degree of shunning the stimulus. This demonstrates that the administration of paclitaxel caused peripheral neuropathy (peripheral nerve hypersensitivity) to develop.

On the other hand, the group receiving 5 mg/kg L-talitol (dotted line with white squares) and the group receiving 6 mg/kg paclitaxel+5 mg/kg L-talitol (broken line with black squares), which received L-talitol, showed results similar to those of the control group. Therefore, it can be concluded that L-talitol suppresses the peripheral neuropathy (peripheral nerve hypersensitivity) induced by paclitaxel. In other words, L-talitol serves as a preventive composition (preventive agent) for peripheral neuropathy that is caused by paclitaxel (anticancer drug).

<Preventive Action of L-Talitol on Peripheral Neuropathy in a Mouse Caused by Vincristine>

The preventive action of L-talitol on hyperesthesia that occurs when an anticancer drug, vincristine, was administered was examined. Examples of hyperesthesia include paresthesia that is caused by a low temperature stimulus and allodynia (severe pain induced by a tactile stimulus that does not usually cause pain) that is caused by a mechanical stimulus. L-talitol was orally administered to a mouse simultaneously with the administration of vincristine, and the following tests (cold plate test and von Frey test) were performed.

(7) Administration of Test Article

Six to seven-week-old female Balb/c mice were used for the tests as with Example 1. All the mice were acclimated for seven days after arrival. Then, the mice were grouped into the following four groups: a control group, a group receiving vincristine, a group receiving L-talitol, and a group receiving vincristine and L-talitol (a group receiving vincristine+L-talitol). Each group consisted of five mice.

On Day 8 of acclimation, the mice in the group receiving vincristine and the group receiving vincristine+L-talitol were intraperitoneally administered with vincristine at a dose of 0.2 mg/kg. This day was designated as Day 0 of administration. Thereafter, these mice were intraperitoneally administered with vincristine at a daily dose of 0.2 mg/kg on Day 7 and Day 14.

The mice in the group receiving L-talitol and the group receiving vincristine+L-talitol were orally administered with L-talitol at a dose of 5 mg/kg daily from Day 0.

These respective groups are referred to as the control group, the group receiving 0.2 mg/kg vincristine, the group receiving 5 mg/kg L-talitol, and the group receiving 0.2 mg/kg vincristine+5 mg/kg L-talitol.

(8) Cold Plate Test

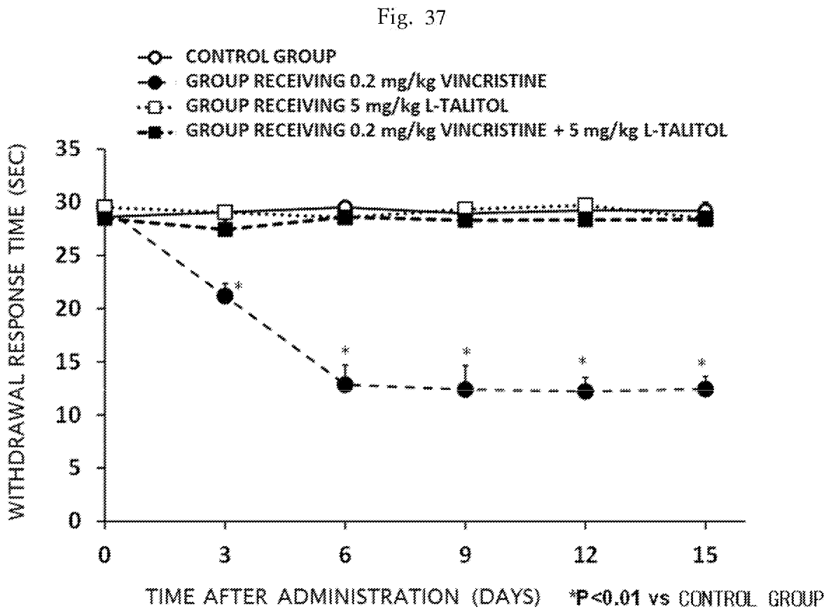

A cold plate test was performed to examine the effect of L-talitol on paresthesia that is caused by a low temperature stimulus. The mice in the four groups described in (7) of the present example (Example 5) were placed on a cold plate set at 10° C., and withdrawal response time (latent time) was measured. It is presumed that a shorter latent time reflects a greater degree of shunning the low temperature stimulus by the cold plate. The results are shown in FIG. 37.

With reference to FIG. 37, the horizontal axis represents the time after administration (days), and the vertical axis represents the average value of the withdrawal response time (seconds) of the mice in each group. The solid line with white circles represents the control group; the broken line with black circles represents the group receiving 0.2 mg/kg vincristine; the dotted line with white squares represents the group receiving 5 mg/kg L-talitol; and the broken line with black squares represents the group receiving 0.2 mg/kg vincristine+5 mg/kg L-talitol. A “*” mark was added to a measurement that was determined to have a significant difference from that of the control group in a test, using a significance level of 1% (in the figure, shown as “*P<0.01 vs control group”).

In the group receiving 0.2 mg/kg vincristine (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg L-talitol (dotted line with white squares), which received only L-talitol, and the group receiving 0.2 mg/kg vincristine+5 mg/kg L-talitol (broken line with black squares), which received vincristine and L-talitol in combination, exhibited a withdrawal response time (latent time) approximately similar to that of the control group (solid line with white circles). The two groups receiving L-talitol also did not show a reduction in the withdrawal response time thereafter. In these two groups, the reduction in the withdrawal response time (latent time) was suppressed compared with the group receiving 0.2 mg/kg vincristine (broken line with black circles).

(9) Von Frey Test

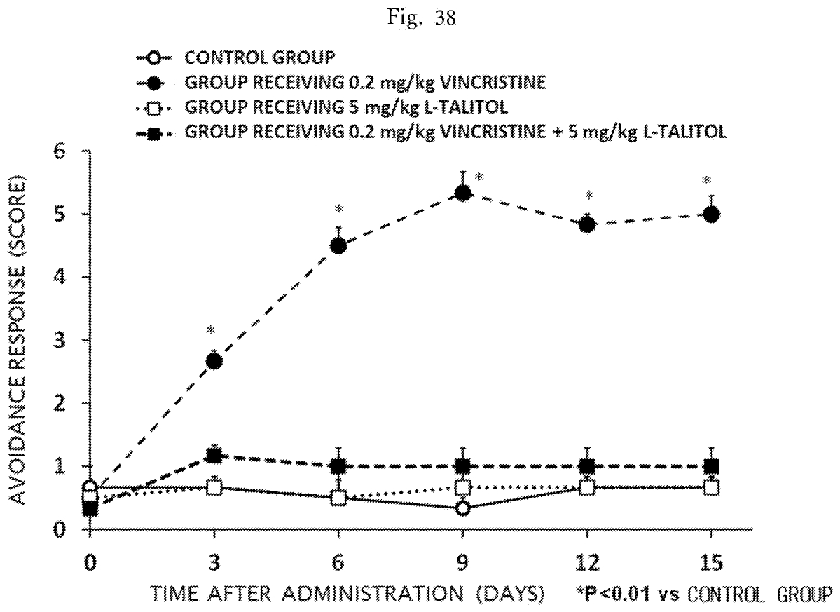

The mice in the four groups described in (7) of the present example (Example 5) were placed in a cage, and the avoidance response frequency (score) was measured by pressing a filament with a bending force of 0.16 g against the back of the hind paw. The results are shown in FIG. 38.

With reference to FIG. 38, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 0.2 mg/kg vincristine; the dotted line with white squares represents the group receiving 5 mg/kg L-talitol; and the broken line with black squares represents the group receiving 0.2 mg/kg vincristine+5 mg/kg L-talitol. A “*” mark was added to a measurement that was determined to have a significant difference from that of the control group in a test, using a significance level of 1% (in the figure, shown as “*P<0.01 vs control group”).

The group receiving 0.2 mg/kg vincristine (broken line with black circles) exhibited a remarkable increase in the avoidance response score compared with the control group (solid line with white circles). On the other hand, the group receiving 5 mg/kg L-talitol (dotted line with white squares), which received only L-talitol, and the group receiving 0.2 mg/kg vincristine+5 mg/kg L-talitol (broken line with black squares), which received L-talitol in combination, exhibited avoidance response scores similar to that of the control group (solid line with white circles) throughout the experimental period. In these two groups, an increase in the avoidance response score was suppressed compared with the group receiving 0.2 mg/kg vincristine (broken line with black circles).

Both the cold plate test (FIG. 37) and the von Frey test (FIG. 38) demonstrated that the group receiving 0.2 mg/kg vincristine (broken line with black circles) exhibited a significant change in both the withdrawal response time and the avoidance response score compared with the control group (solid line with white circles), the change being a shift to a greater degree of shunning the stimulus. This demonstrates that the administration of vincristine caused peripheral neuropathy (peripheral nerve hypersensitivity) to develop.

On the other hand, the group receiving 5 mg/kg L-talitol (dotted line with white squares) and the group receiving 0.2 mg/kg vincristine+5 mg/kg L-talitol (broken line with black squares), which received L-talitol, showed results similar to those of the control group. Therefore, it can be concluded that L-talitol suppresses the peripheral neuropathy (peripheral nerve hypersensitivity) induced by vincristine. In other words, L-talitol serves as a preventive composition (preventive agent) for peripheral neuropathy that is caused by vincristine (anticancer drug).

<Preventive Action of L-Talitol on Peripheral Neuropathy in a Mouse Caused by Bortezomib>

The preventive action of L-talitol on hyperesthesia that occurs when an anticancer drug, bortezomib, was administered was examined. Examples of hyperesthesia include paresthesia that is caused by a low temperature stimulus and allodynia (severe pain induced by a tactile stimulus that does not usually cause pain) that is caused by a mechanical stimulus. L-talitol was orally administered to a mouse simultaneously with the administration of bortezomib, and the following tests (cold plate test and von Frey test) were performed.

(10) Administration of Test Article

Six to seven-week-old female Balb/c mice were used for the tests as with Example 1. All the mice were acclimated for seven days after arrival. Then, the mice were grouped into the following four groups: a control group, a group receiving bortezomib, a group receiving L-talitol, and a group receiving bortezomib and L-talitol (a group receiving bortezomib+L-talitol). Each group consisted of five mice.

On Day 8 of acclimation, the mice in the group receiving bortezomib and the group receiving bortezomib+L-talitol were intraperitoneally administered with bortezomib at a dose of 1 mg/kg. This day was designated as Day 0 of administration. Thereafter, these mice were intraperitoneally administered with bortezomib at a daily dose of 1 mg/kg on Day 7 and Day 14.

The mice in the group receiving L-talitol and the group receiving bortezomib+L-talitol were orally administered with L-talitol at a dose of 5 mg/kg daily from Day 0.

These respective groups are referred to as the control group, the group receiving 1 mg/kg bortezomib, the group receiving 5 mg/kg L-talitol, and the group receiving 1 mg/kg bortezomib+5 mg/kg L-talitol.

(11) Cold Plate Test

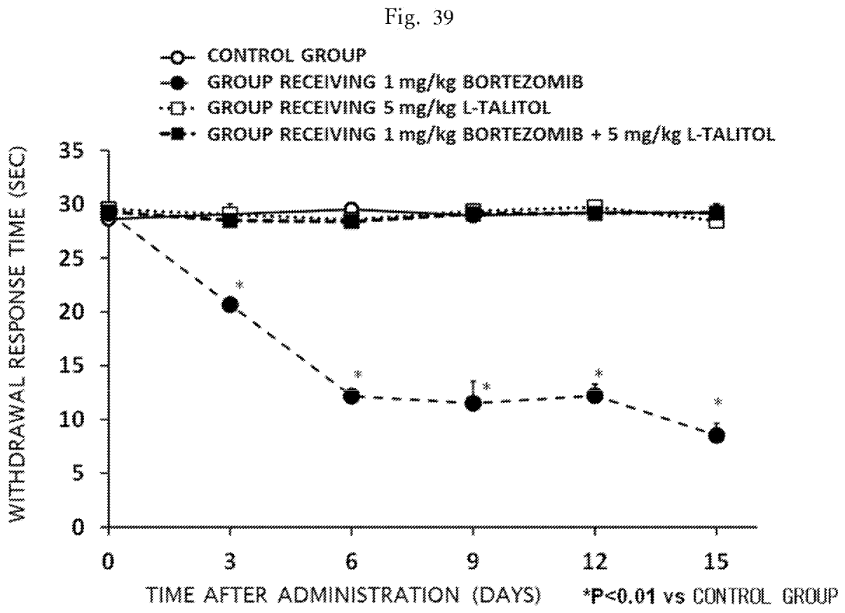

A cold plate test was performed to examine the effect of L-talitol on paresthesia that is caused by a low temperature stimulus. The mice in the four groups described in (10) of the present example (Example 5) were placed on a cold plate set at 10° C., and withdrawal response time (latent time) was measured. It is presumed that a shorter latent time reflects a greater degree of shunning the low temperature stimulus by the cold plate. The results are shown in FIG. 39.

With reference to FIG. 39, the horizontal axis represents the time after administration (days), and the vertical axis represents the average value of the withdrawal response time (seconds) of the mice in each group. The solid line with white circles represents the control group; the broken line with black circles represents the group receiving 1 mg/kg bortezomib; the dotted line with white squares represents the group receiving 5 mg/kg L-talitol; and the broken line with black squares represents the group receiving 1 mg/kg bortezomib+5 mg/kg L-talitol. A “*” mark was added to a measurement that was determined to have a significant difference from that of the control group in a test, using a significance level of 1% (in the figure, shown as “*P<0.01 vs control group”).

In the group receiving 1 mg/kg bortezomib (broken line with black circles), the withdrawal response time (latent time) when given a cold stimulus by the cold plate was reduced from Day 3 to Day 6 of the test, and thereafter, the withdrawal response time remained constant. On the other hand, the group receiving 5 mg/kg L-talitol (dotted line with white squares), which received only L-talitol, and the group receiving 1 mg/kg bortezomib+5 mg/kg L-talitol (broken line with black squares), which received bortezomib and L-talitol in combination, exhibited a withdrawal response time (latent time) approximately similar to that of the control group (solid line with white circles). The two groups receiving L-talitol also did not show a reduction in the withdrawal response time thereafter. In these two groups, the reduction in the withdrawal response time (latent time) was suppressed compared with the group receiving 1 mg/kg bortezomib (broken line with black circles).

(12) Von Frey Test

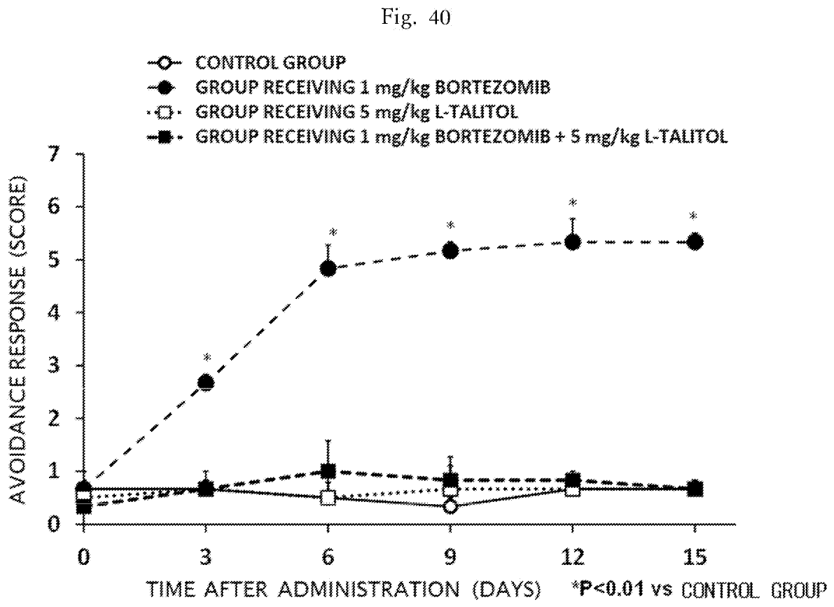

The mice in the four groups described in (10) of the present example (Example 5) were placed in a cage, and the avoidance response frequency (score) was measured by pressing a filament with a bending force of 0.16 g against the back of the hind paw. The results are shown in FIG. 40.

With reference to FIG. 40, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving 1 mg/kg bortezomib; the dotted line with white squares represents the group receiving 5 mg/kg L-talitol; and the broken line with black squares represents the group receiving 1 mg/kg bortezomib+5 mg/kg L-talitol. A “*” mark was added to a measurement that was determined to have a significant difference from that of the control group in a test, using a significance level of 1% (in the figure, shown as “*P<0.01 vs control group”).

The group receiving 1 mg/kg bortezomib (broken line with black circles) exhibited a remarkable increase in the avoidance response score compared with the control group (solid line with white circles). On the other hand, the group receiving 5 mg/kg L-talitol (dotted line with white squares), which received only L-talitol, and the group receiving 1 mg/kg bortezomib+5 mg/kg L-talitol (broken line with black squares), which received L-talitol in combination, exhibited avoidance response scores similar to that of the control group (solid line with white circles) throughout the experimental period. In these two groups, an increase in the avoidance response score was suppressed compared with the group receiving 1 mg/kg bortezomib (broken line with black circles).

Both the cold plate test (FIG. 39) and the von Frey test (FIG. 40) demonstrated that the group receiving 1 mg/kg bortezomib (broken line with black circles) exhibited a significant change in both the withdrawal response time and the avoidance response score compared with the control group (solid line with white circles), the change being a shift to a greater degree of shunning the stimulus. This demonstrates that the administration of bortezomib caused peripheral neuropathy (peripheral nerve hypersensitivity) to develop.

On the other hand, the group receiving 5 mg/kg L-talitol (dotted line with white squares) and the group receiving 1 mg/kg bortezomib+5 mg/kg L-talitol (broken line with black squares), which received L-talitol, showed results similar to those of the control group. Therefore, it can be concluded that L-talitol suppresses the peripheral neuropathy (peripheral nerve hypersensitivity) induced by bortezomib. In other words, L-talitol serves as a preventive composition (preventive agent) for peripheral neuropathy that is caused by bortezomib (anticancer drug).

### (Example 6) L-Talitol-Containing Therapeutic Agent for Peripheral Neuropathy that is Caused by Administration of Anticancer Drug

### <Therapeutic Action of L-Talitol on Peripheral Neuropathy in a Mouse Caused by Oxaliplatin>

It was found that L-talitol is capable of preventing the peripheral neuropathy that is caused by oxaliplatin. Therefore, whether L-talitol had a therapeutic action of alleviating peripheral neuropathy after the development of peripheral neuropathy via the intake of an anticancer drug was subsequently examined.

(1) Administration of Test Article

Six to seven-week-old female Balb/c mice were used for the tests as with Example 1. All the mice were acclimated for seven days after arrival. Then, the mice were grouped into the following four groups: a control group, a group receiving oxaliplatin, a group receiving L-talitol, and a group receiving oxaliplatin and L-talitol (a group receiving oxaliplatin+L-talitol). Each group consisted of five mice.

On Day 8 of acclimation, the mice in the group receiving oxaliplatin and the group receiving oxaliplatin+L-talitol were intraperitoneally administered with oxaliplatin at a dose of 6 mg/kg. This day was designated as the first day of the administration (Day 0). Thereafter, these mice were intraperitoneally administered with oxaliplatin at the same dose on Day 7 and Day 14 for a total of 3 doses.

The mice in the group receiving 100 mg/kg L-talitol were orally administered with L-talitol at a dose of 100 mg/kg daily from Day 0. The mice in the group receiving oxaliplatin+100 mg/kg L-talitol were orally administered with L-talitol at a dose of 100 mg/kg daily from Day 6 of administration.

(2) Cold Plate Test

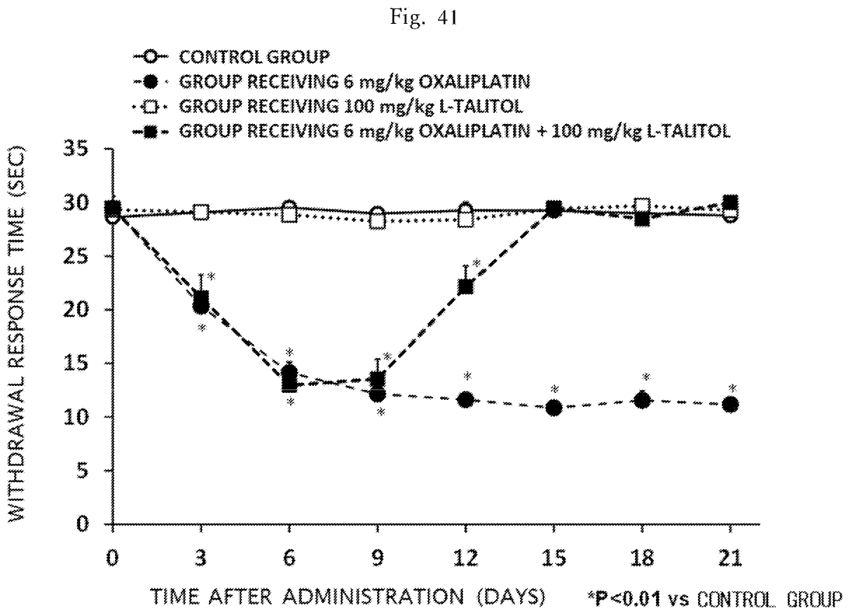

A test was performed on the mice in the four groups described in (1) of the present example (Example 6) to examine the effect of L-talitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 41.

With reference to FIG. 41, the horizontal axis represents the time after administration (days), and the vertical axis represents the average value of the withdrawal response time (seconds) of the mice in each group. The solid line with white circles represents the control group; the broken line with black circles represents the group receiving 6 mg/kg oxaliplatin; the dotted line with white squares represents the group receiving 100 mg/kg L-talitol; and the broken line with black squares represents the group receiving 6 mg/kg oxaliplatin+100 mg/kg L-talitol. A “*” mark was added to a measurement that was determined to have a significant difference from that of the control group in a test, using a significance level of 1% (in the figure, shown as “*P<0.01 vs control group”).

With reference to FIG. 41, in the groups receiving oxaliplatin other than the control group and the group receiving L-talitol, a uniform reduction in the withdrawal response time (latent time) was observed by Day 6 after administration. However, the latent time tended to become longer from Day 9 onwards in the group receiving 6 mg/kg oxaliplatin+100 mg/kg L-talitol. On Day 15, the group receiving 6 mg/kg oxaliplatin+100 mg/kg L-talitol had a withdrawal response time similar to that of the control group (solid line with white circles).

The withdrawal response time (latent time) was greatly reduced on Day 6 of administration in the two groups receiving oxaliplatin (the group receiving 6 mg/kg oxaliplatin, and the group receiving 6 mg/kg oxaliplatin+100 mg/kg L-talitol). Thus, it is presumed that the mice in these groups developed peripheral neuropathy (peripheral nerve hypersensitivity).

After that, the group receiving L-talitol exhibited a significantly longer withdrawal response time (latent time) compared with the group receiving 6 mg/kg oxaliplatin and no L-talitol. This result signifies that the peripheral neuropathy (peripheral nerve hypersensitivity) that had developed beforehand was ameliorated by taking L-talitol. Thus, it was found that L-talitol serves as a therapeutic composition (therapeutic agent) for peripheral neuropathy (peripheral nerve hypersensitivity).

(3) Von Frey Test

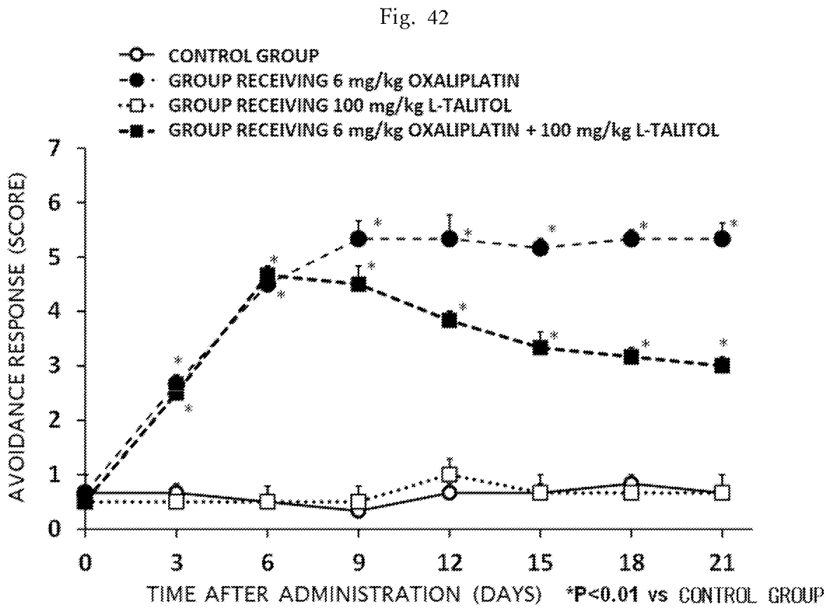

For the mice in the four groups described in (1) of the present example (Example 6), the avoidance response frequency (score) was measured by pressing a filament with a bending force of 0.16 g against the back of the hind paw. The results are shown in FIG. 42.

With reference to FIG. 42, the horizontal axis represents the time after administration (days), and the vertical axis represents the average value of the avoidance responses (score) of the mice in each group. The solid line with white circles represents the control group; the broken line with black circles represents the group receiving 6 mg/kg oxaliplatin; the dotted line with white squares represents the group receiving 100 mg/kg L-talitol; and the broken line with black squares represents the group receiving 6 mg/kg oxaliplatin+100 mg/kg L-talitol. A “*” mark was added to a measurement that was determined to have a significant difference from that of the control group in a test, using a significance level of 1% (in the figure, shown as “*P<0.01 vs control group”).

The group receiving oxaliplatin (broken line with black circles) exhibited a remarkable increase in the avoidance response score compared with the control group (solid line with white circles). Thus, it is presumed that the mice in the group receiving oxaliplatin developed peripheral neuropathy (peripheral nerve hypersensitivity). On the other hand, the group receiving 6 mg/kg oxaliplatin+100 mg/kg L-talitol (broken line with black squares), which received L-talitol in combination, maintained a score clearly lower than that of the group receiving oxaliplatin (broken line with black circles) from Day 12 onwards. Thus, it was found that L-talitol also serves as a therapeutic composition (therapeutic agent) for peripheral neuropathy (peripheral nerve hypersensitivity).

<Therapeutic Action of L-Talitol on Peripheral Neuropathy in a Mouse Caused by Paclitaxel>

It was found that L-talitol is capable of preventing the peripheral neuropathy that is caused by paclitaxel. Therefore, whether L-talitol had a therapeutic action of alleviating peripheral neuropathy after the development of peripheral neuropathy via the intake of an anticancer drug was subsequently examined.

(4) Administration of Test Article

Six to seven-week-old female Balb/c mice were used for the tests as with Example 1. All the mice were acclimated for seven days after arrival. Then, the mice were grouped into the following four groups: a control group, a group receiving paclitaxel, a group receiving L-talitol, and a group receiving paclitaxel and L-talitol (a group receiving paclitaxel+L-talitol). Each group consisted of five mice.

On Day 8 of acclimation, the mice in the group receiving paclitaxel and the group receiving paclitaxel+L-talitol were intraperitoneally administered with paclitaxel at a dose of 6 mg/kg. This day was designated as the first day of the administration (Day 0). Thereafter, these mice were intraperitoneally administered with paclitaxel at the same dose on Day 7 and Day 14 for a total of 3 doses.

The mice in the group receiving 100 mg/kg L-talitol were orally administered with L-talitol at a dose of 100 mg/kg daily from Day 0. The mice in the group receiving paclitaxel+100 mg/kg L-talitol were orally administered with L-talitol at a dose of 100 mg/kg daily from Day 6 of administration.

(5) Cold Plate Test

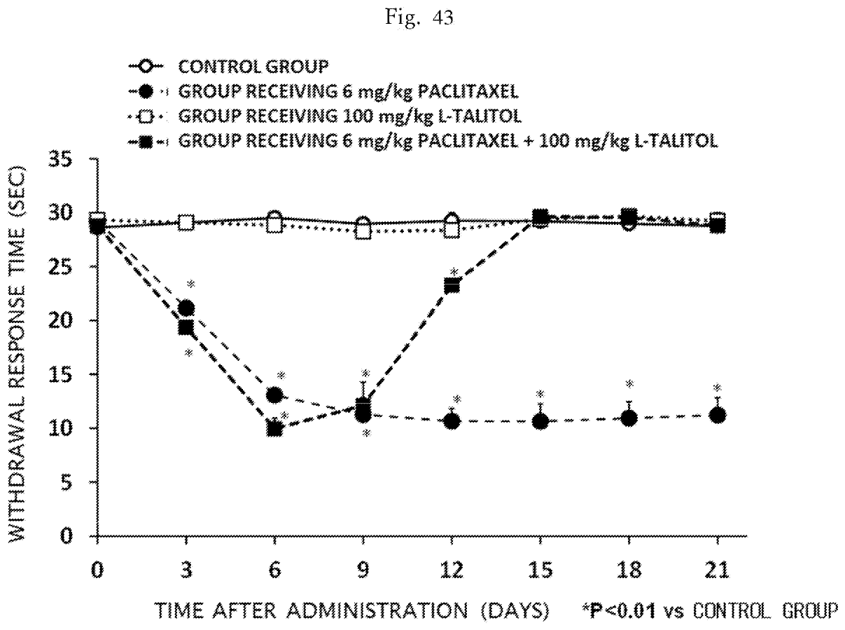

A test was performed on the mice in the four groups described in (4) of the present example (Example 6) to examine the effect of L-talitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 43.

With reference to FIG. 43, the horizontal axis represents the time after administration (days), and the vertical axis represents the average value of the withdrawal response time (seconds) of the mice in each group. The solid line with white circles represents the control group; the broken line with black circles represents the group receiving 6 mg/kg paclitaxel; the dotted line with white squares represents the group receiving 100 mg/kg L-talitol; and the broken line with black squares represents the group receiving 6 mg/kg paclitaxel+100 mg/kg L-talitol. A “*” mark was added to a measurement that was determined to have a significant difference from that of the control group in a test, using a significance level of 1% (in the figure, shown as “*P<0.01 vs control group”).

With reference to FIG. 43, in the two groups receiving paclitaxel other than the control group and the group receiving L-talitol, a uniform reduction in the withdrawal response time (latent time) was observed by Day 6 after administration. However, the latent time tended to become longer from Day 9 onwards in the group receiving 6 mg/kg paclitaxel+100 mg/kg L-talitol. On Day 12, the group receiving 6 mg/kg paclitaxel+100 mg/kg L-talitol had a significantly longer withdrawal response time (latent time) compared with the group receiving 6 mg/kg paclitaxel.

The withdrawal response time (latent time) was greatly reduced on Day 6 of administration in the two groups receiving paclitaxel (the group receiving 6 mg/kg paclitaxel, and the group receiving 6 mg/kg paclitaxel+100 mg/kg L-talitol). Thus, it is presumed that the mice in these groups developed peripheral neuropathy (peripheral nerve hypersensitivity).

After that, the groups receiving L-talitol exhibited a significantly longer withdrawal response time (latent time) compared with the group receiving 6 mg/kg paclitaxel and no L-talitol. This result signifies that the peripheral neuropathy (peripheral nerve hypersensitivity) that had developed beforehand was ameliorated by taking L-talitol. Thus, it was found that L-talitol also serves as a therapeutic composition (therapeutic agent) for peripheral neuropathy (peripheral nerve hypersensitivity).

(6) Von Frey Test

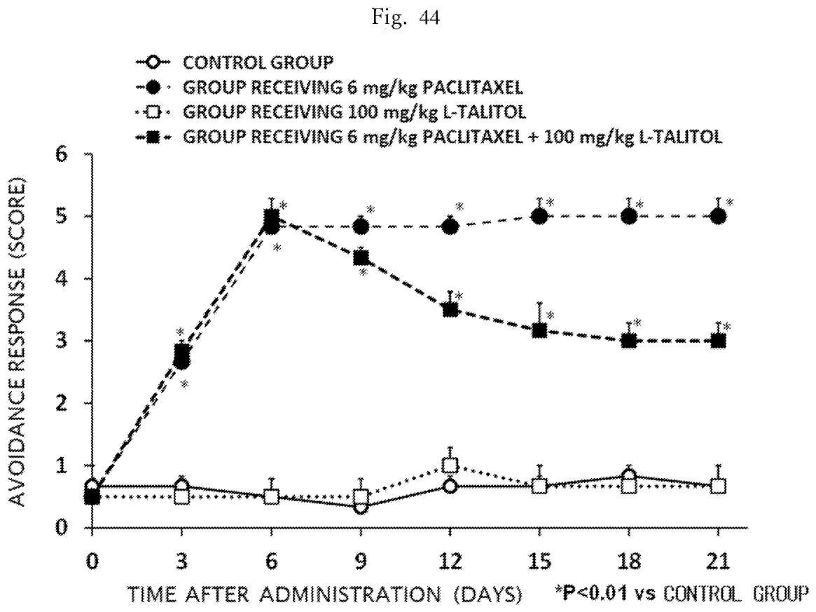

For the mice in the four groups described in (4) of the present example (Example 6), the avoidance response frequency (score) was measured by pressing a filament with a bending force of 0.16 g against the back of the hind paw. The results are shown in FIG. 44.

With reference to FIG. 44, the horizontal axis represents the time after administration (days), and the vertical axis represents the average value of the avoidance responses (score) of the mice in each group. The solid line with white circles represents the control group; the broken line with black circles represents the group receiving 6 mg/kg paclitaxel; the dotted line with white squares represents the group receiving 100 mg/kg L-talitol; and the broken line with black squares represents the group receiving 6 mg/kg paclitaxel+100 mg/kg L-talitol. A “*” mark was added to a measurement that was determined to have a significant difference from that of the control group in a test, using a significance level of 1% (in the figure, shown as “*P<0.01 vs control group”).

The group receiving paclitaxel (broken line with black circles) exhibited a remarkable increase in the avoidance response score compared with the control group (solid line with white circles). Thus, it is presumed that the mice in the group receiving paclitaxel developed peripheral neuropathy (peripheral nerve hypersensitivity). On the other hand, the group receiving 6 mg/kg paclitaxel+100 mg/kg L-talitol (broken line with black squares), which received L-talitol in combination, maintained a score clearly lower than that of the group receiving paclitaxel (broken line with black circles) from Day 12 onwards. Thus, it was found that L-talitol also serves as a therapeutic composition (therapeutic agent) for peripheral neuropathy (peripheral nerve hypersensitivity).

<Therapeutic Action of L-Talitol on Peripheral Neuropathy in a Mouse Caused by Vincristine>

It was found that L-talitol is capable of preventing the peripheral neuropathy that is caused by vincristine. Therefore, whether L-talitol had a therapeutic action of alleviating peripheral neuropathy after the development of peripheral neuropathy via the intake of an anticancer drug was subsequently examined.

(7) Administration of Test Article

Six to seven-week-old female Balb/c mice were used for the tests as with Example 1. All the mice were acclimated for seven days after arrival. Then, the mice were grouped into the following four groups: a control group, a group receiving vincristine, a group receiving L-talitol, and a group receiving vincristine and L-talitol (a group receiving vincristine+L-talitol). Each group consisted of five mice.

On Day 8 of acclimation, the mice in the group receiving vincristine and the group receiving vincristine+L-talitol were intraperitoneally administered with vincristine at a dose of 0.2 mg/kg. This day was designated as the first day of the administration (Day 0). Thereafter, these mice were intraperitoneally administered with vincristine at the same dose on Day 7 and Day 14 for a total of 3 doses.

The mice in the group receiving 100 mg/kg L-talitol were orally administered with L-talitol at a dose of 100 mg/kg daily from Day 0. The mice in the group receiving vincristine+100 mg/kg L-talitol were orally administered with L-talitol at a dose of 100 mg/kg daily from Day 6 of administration.

(8) Cold Plate Test

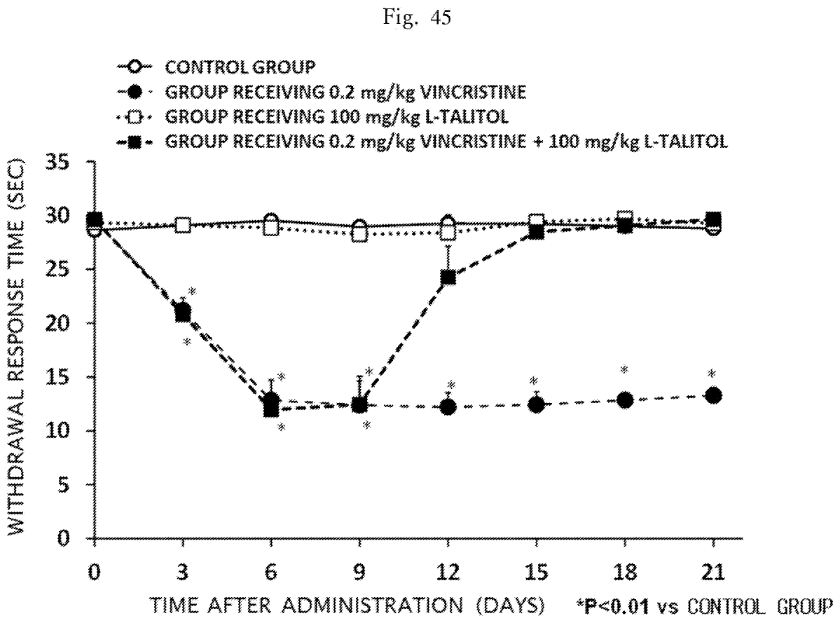

A test was performed on the mice in the four groups described in (7) of the present example (Example 6) to examine the effect of L-talitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 45.

With reference to FIG. 45, the horizontal axis represents the time after administration (days), and the vertical axis represents the average value of the withdrawal response time (seconds) of the mice in each group. The solid line with white circles represents the control group; the broken line with black circles represents the group receiving 0.2 mg/kg vincristine; the dotted line with white squares represents the group receiving 100 mg/kg L-talitol; and the broken line with black squares represents the group receiving 0.2 mg/kg vincristine+100 mg/kg L-talitol. A “*” mark was added to a measurement that was determined to have a significant difference from that of the control group in a test, using a significance level of 1% (in the figure, shown as “*P<0.01 vs control group”).

With reference to FIG. 45, in the two groups receiving vincristine other than the control group and the group receiving L-talitol, a uniform reduction in the withdrawal response time (latent time) was observed by Day 6 after administration. However, the latent time tended to become longer from Day 9 onwards in the group receiving 0.2 mg/kg vincristine+100 mg/kg L-talitol. On Day 12, the group receiving 0.2 mg/kg vincristine+100 mg/kg L-talitol had a significantly longer withdrawal response time (latent time) compared with the group receiving 0.2 mg/kg vincristine.

The withdrawal response time (latent time) was greatly reduced on Day 6 of administration in the two groups receiving vincristine (the group receiving 0.2 mg/kg vincristine, and the group receiving 0.2 mg/kg vincristine+100 mg/kg L-talitol). Thus, it is presumed that the mice in these groups developed peripheral neuropathy (peripheral nerve hypersensitivity).

After that, the groups receiving L-talitol exhibited a significantly longer withdrawal response time (latent time) compared with the group receiving 0.2 mg/kg vincristine and no L-talitol. This result signifies that the peripheral neuropathy (peripheral nerve hypersensitivity) that had developed beforehand was ameliorated by taking L-talitol. Thus, it was found that L-talitol also serves as a therapeutic composition (therapeutic agent) for peripheral neuropathy (peripheral nerve hypersensitivity).

(9) Von Frey Test

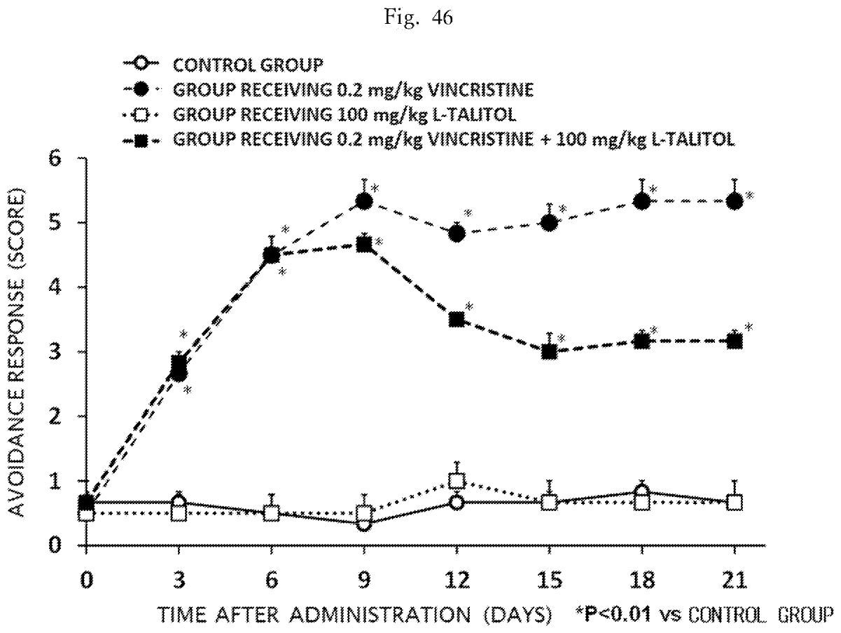

For the mice in the four groups described in (7) of the present example (Example 6), the avoidance response frequency (score) was measured by pressing a filament with a bending force of 0.16 g against the back of the hind paw. The results are shown in FIG. 46.

With reference to FIG. 46, the horizontal axis represents the time after administration (days), and the vertical axis represents the average value of the avoidance responses (score) of the mice in each group. The solid line with white circles represents the control group; the broken line with black circles represents the group receiving 0.2 mg/kg vincristine; the dotted line with white squares represents the group receiving 100 mg/kg L-talitol; and the broken line with black squares represents the group receiving 0.2 mg/kg vincristine+100 mg/kg L-talitol. A “*” mark was added to a measurement that was determined to have a significant difference from that of the control group in a test, using a significance level of 1% (in the figure, shown as “*P<0.01 vs control group”).

The group receiving vincristine (broken line with black circles) exhibited a remarkable increase in the avoidance response score compared with the control group (solid line with white circles). Thus, it is presumed that the mice in the group receiving vincristine developed peripheral neuropathy (peripheral nerve hypersensitivity). On the other hand, the group receiving 0.2 mg/kg vincristine+100 mg/kg L-talitol (broken line with black squares), which received L-talitol in combination, maintained a score clearly lower than that of the group receiving vincristine (broken line with black circles) from Day 12 onwards. Thus, it was found that L-talitol also serves as a therapeutic composition (therapeutic agent) for peripheral neuropathy (peripheral nerve hypersensitivity).

<Therapeutic Action of L-Talitol on Peripheral Neuropathy in a Mouse Caused by Bortezomib>

It was found that L-talitol is capable of preventing the peripheral neuropathy that is caused by bortezomib. Therefore, whether L-talitol had a therapeutic action of alleviating peripheral neuropathy after the development of peripheral neuropathy via the intake of an anticancer drug was subsequently examined.

(10) Administration of Test Article

Six to seven-week-old female Balb/c mice were used for the tests as with Example 1. All the mice were acclimated for seven days after arrival. Then, the mice were grouped into the following four groups: a control group, a group receiving bortezomib, a group receiving L-talitol, and a group receiving bortezomib and L-talitol (a group receiving bortezomib+L-talitol). Each group consisted of five mice.

On Day 8 of acclimation, the mice in the group receiving bortezomib and the group receiving bortezomib+L-talitol were intraperitoneally administered with bortezomib at a dose of 1 mg/kg. This day was designated as the first day of the administration (Day 0). Thereafter, these mice were intraperitoneally administered with bortezomib at the same dose on Day 7 and Day 14 for a total of 3 doses.

The mice in the group receiving 100 mg/kg L-talitol were orally administered with L-talitol at a dose of 100 mg/kg daily from Day 0. The mice in the group receiving bortezomib+100 mg/kg L-talitol were orally administered with L-talitol at a dose of 100 mg/kg daily from Day 6 of administration.

(11) Cold Plate Test

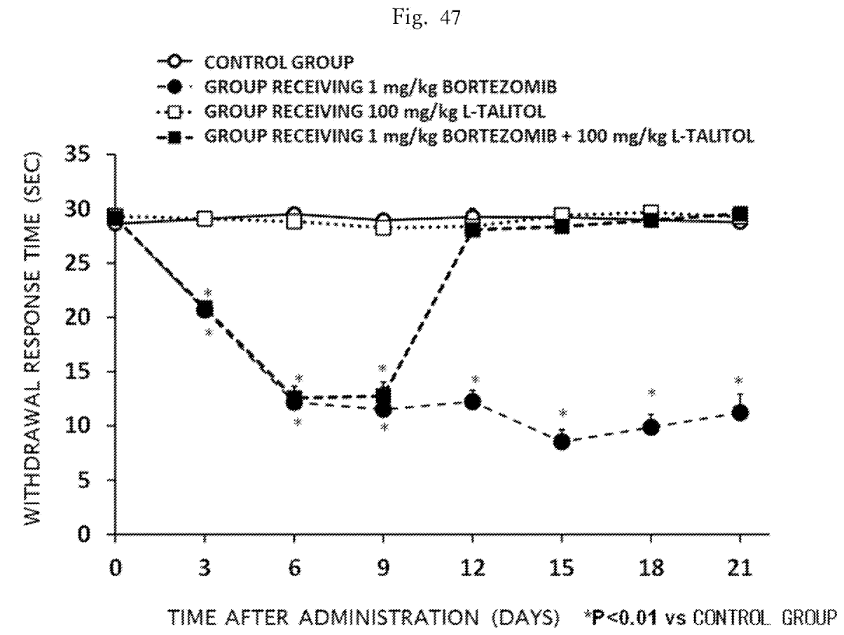

A test was performed on the mice in the four groups described in (10) of the present example (Example 6) to examine the effect of L-talitol on paresthesia that is caused by a low temperature stimulus. The mice in respective groups were placed on a cold plate set at 10° C., and withdrawal response time (latent time) was measured. It is presumed that a shorter latent time reflects a greater degree of shunning the low temperature stimulus by the cold plate. The results are shown in FIG. 47.

With reference to FIG. 47, the horizontal axis represents the time after administration (days), and the vertical axis represents the average value of the withdrawal response time (seconds) of the mice in each group. The solid line with white circles represents the control group; the broken line with black circles represents the group receiving 1 mg/kg bortezomib; the dotted line with white squares represents the group receiving 100 mg/kg L-talitol; and the broken line with black squares represents the group receiving 1 mg/kg bortezomib+100 mg/kg L-talitol. A “*” mark was added to a measurement that was determined to have a significant difference from that of the control group in a test, using a significance level of 1% (in the figure, shown as “*P<0.01 vs control group”).

With reference to FIG. 47, in the two groups receiving vincristine other than the control group and the group receiving L-talitol, a uniform reduction in the withdrawal response time (latent time) was observed by Day 6 after administration. However, the latent time tended to become longer from Day 9 onwards in the group receiving 1 mg/kg bortezomib+100 mg/kg L-talitol. On Day 12, the group receiving 1 mg/kg bortezomib+100 mg/kg L-talitol had a significantly longer withdrawal response time (latent time) compared with the group receiving 1 mg/kg bortezomib.

The withdrawal response time (latent time) was greatly reduced on Day 6 of administration in the two groups receiving bortezomib (the group receiving 1 mg/kg bortezomib, and the group receiving 1 mg/kg bortezomib+100 mg/kg L-talitol). Thus, it is presumed that the mice in these groups developed peripheral neuropathy (peripheral nerve hypersensitivity).

After that, the groups receiving L-talitol exhibited a significantly longer withdrawal response time (latent time) compared with the group receiving 1 mg/kg bortezomib and no L-talitol. This result signifies that the peripheral neuropathy (peripheral nerve hypersensitivity) that had developed beforehand was ameliorated by taking L-talitol. Thus, it was found that L-talitol also serves as a therapeutic composition (therapeutic agent) for peripheral neuropathy (peripheral nerve hypersensitivity).

(12) Von Frey Test

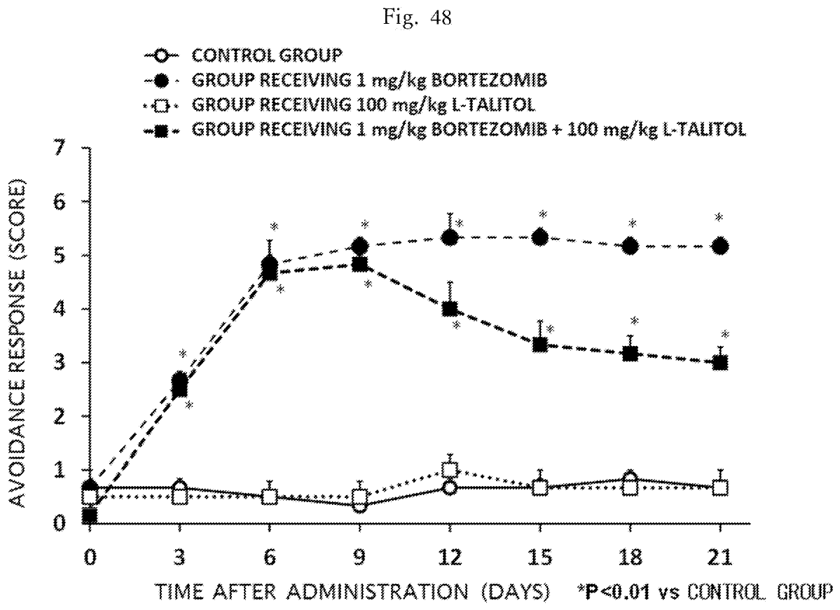

For the mice in the four groups described in (10) of the present example (Example 6), the avoidance response frequency (score) was measured by pressing a filament with a bending force of 0.16 g against the back of the hind paw. The results are shown in FIG. 48.

With reference to FIG. 48, the horizontal axis represents the time after administration (days), and the vertical axis represents the average value of the avoidance responses (score) of the mice in each group. The solid line with white circles represents the control group; the broken line with black circles represents the group receiving 1 mg/kg bortezomib; the dotted line with white squares represents the group receiving 100 mg/kg L-talitol; and the broken line with black squares represents the group receiving 1 mg/kg bortezomib+100 mg/kg L-talitol. A “*” mark was added to a measurement that was determined to have a significant difference from that of the control group in a test, using a significance level of 1% (in the figure, shown as “*P<0.01 vs control group”).

The group receiving bortezomib (broken line with black circles) exhibited a remarkable increase in the avoidance response score compared with the control group (solid line with white circles). Thus, it is presumed that the mice in the group receiving bortezomib developed peripheral neuropathy (peripheral nerve hypersensitivity). On the other hand, the group receiving 1 mg/kg bortezomib+100 mg/kg L-talitol (broken line with black squares), which received L-talitol in combination, maintained a score clearly lower than that of the group receiving bortezomib (broken line with black circles) from Day 12 onwards. Thus, it was found that L-talitol also serves as a therapeutic composition (therapeutic agent) for peripheral neuropathy (peripheral nerve hypersensitivity).

### (Example 7) Xylitol-Containing Preventive Agent for Diabetic Peripheral Neuropathy

### <Preventive Action of Xylitol on Diabetic Peripheral Neuropathy in a Mouse Induced by Streptozotocin>

The preventive effect of xylitol on hyperesthesia that occurs in diabetic peripheral neuropathy was examined. Examples of hyperesthesia include paresthesia that is caused by a low temperature stimulus and allodynia (severe pain induced by a tactile stimulus that does not usually cause pain) that is caused by a mechanical stimulus. Xylitol was orally administered as a test drug to a mouse simultaneously with the administration of streptozotocin, and the following tests (cold plate test and von Frey test) were performed.

(1) Administration of Test Drug

Six to seven-week-old male C57BL/6J mice were used for the tests. All the mice were acclimated for seven days after arrival. Then, the mice were grouped into the following four groups: a control group, a group receiving streptozotocin, a group receiving 1 mg/kg xylitol, and a group receiving streptozotocin+1 mg/kg xylitol. Each group consisted of nine mice.

On Day 8 of acclimation, the mice in the group receiving streptozotocin and the group receiving streptozotocin+1 mg/kg xylitol were administered with streptozotocin at a dose of 200 mg/kg. A massive dose of streptozotocin destroys pancreatic cells in the mice. As a result, insulin is no longer secreted, and thus, diabetes can be developed in the mice. This day was designated as the first day of the administration (Day 0). Note that streptozotocin was administered only on the first day of administration.

The mice in the group receiving 1 mg/kg xylitol and the group receiving streptozotocin+1 mg/kg xylitol were orally administered with xylitol at a dose of 1 mg/kg daily from Day 0.

(2) Cold Plate Test

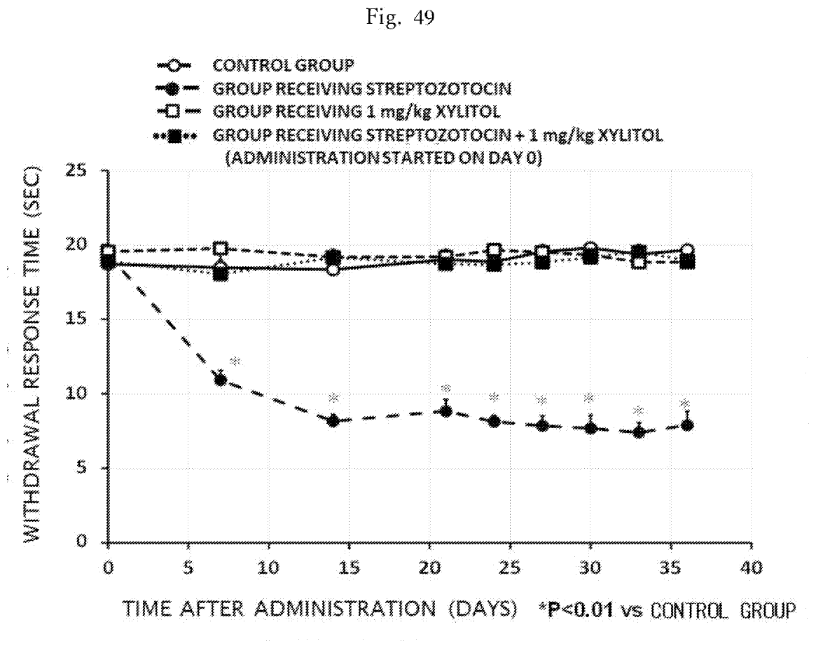

A cold plate test was performed to examine the effect of xylitol on paresthesia that is caused by a low temperature stimulus. The mice in the four groups described in (1) of the present example (Example 7) were placed on a cold plate set at 4° C., and withdrawal response time (latent time) was measured. It is presumed that a shorter latent time reflects a greater degree of shunning the low temperature stimulus by the cold plate. The results are shown in FIG. 49.

With reference to FIG. 49, the horizontal axis represents the time after administration (days), and the vertical axis represents the average value of the withdrawal response time (seconds) of the mice in each group. The solid line with white circles represents the control group; the broken line with black circles represents the group receiving streptozotocin; the broken line with white squares represents the group receiving 1 mg/kg xylitol; and the dotted line with black squares represents the group receiving streptozotocin+1 mg/kg xylitol. A “*” mark was added to a measurement that was determined to have a significant difference from that of the control group in a test, using a significance level of 1% (in the figure, shown as “*P<0.01 vs control group”).

The control group (solid line with white circles) exhibited a stable withdrawal response time ranging from 18 seconds to 20 seconds throughout the experimental period. In the group receiving streptozotocin (broken line with black circles), the withdrawal response time was reduced from Day 7. On Day 14, the withdrawal response time (latent time) was greatly reduced compared with the control group (solid line with white circles).

On the other hand, the group receiving 1 mg/kg xylitol (broken line with white squares), which received only xylitol, and the group receiving streptozotocin+1 mg/kg xylitol (dotted line with black squares), which received xylitol in combination with streptozotocin, exhibited a response time similar to that of the control group (solid line with white circles) throughout the experimental period, with no reduction in the withdrawal response time. In these groups, the reduction in the latent time was suppressed compared with the group receiving streptozotocin (broken line with black circles).

(3) Von Frey Test

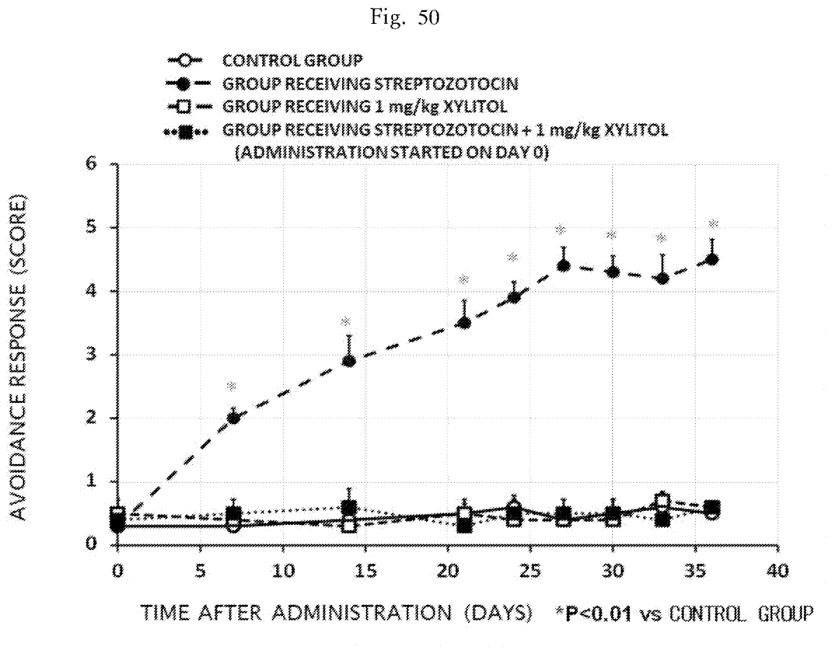

The mice in the four groups described in (1) of the present example (Example 7) were placed in a cage, and the avoidance response frequency (score) was measured by pressing a filament with a bending force of 0.16 g against the back of the hind paw. The results are shown in FIG. 50.

With reference to FIG. 50, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving streptozotocin; the broken line with white squares represents the group receiving 1 mg/kg xylitol; and the dotted line with black squares represents the group receiving streptozotocin+1 mg/kg xylitol. A “*” mark was added to a measurement that was determined to have a significant difference from that of the control group in a test, using a significance level of 1% (in the figure, shown as “*P<0.01 vs control group”).

The control group (solid line with white circles) exhibited a stable score of one or less throughout the experimental period. In the group receiving streptozotocin (broken line with black circles), the avoidance response score was increased from Day 7. On the other hand, the group receiving 1 mg/kg xylitol (broken line with white squares) and the group receiving streptozotocin+1 mg/kg xylitol (dotted line with black squares), which received xylitol, exhibited avoidance response scores similar to that of the control group (solid line with white circles) throughout the experimental period. In these two groups, an increase in the avoidance response score was suppressed compared with the group receiving streptozotocin (broken line with black circles).

Both the cold plate test (FIG. 49) and the von Frey test (FIG. 50) demonstrated that the group receiving streptozotocin (broken line with black circles) exhibited a significant change in both the avoidance response score and the withdrawal response time compared with the control group (solid line with white circles), the change being a shift to a greater degree of shunning the stimulus. This demonstrates that the administration of streptozotocin resulted in the onset of diabetes in the mice, causing peripheral neuropathy (peripheral nerve hypersensitivity) to develop.

On the other hand, the group receiving 1 mg/kg xylitol (broken line with white squares) and the group receiving streptozotocin+1 mg/kg xylitol (broken line with black squares), which received xylitol, showed results similar to those of the control group (solid line with white circles). Therefore, it can be concluded that xylitol suppresses the peripheral neuropathy (peripheral nerve hypersensitivity) induced by streptozotocin-induced diabetes. In other words, xylitol serves as a preventive composition (preventive agent) for peripheral neuropathy that is caused by diabetes.

### (Example 8) Xylitol-Containing Therapeutic Agent for Diabetic Peripheral Neuropathy

### <Therapeutic Action of Xylitol on Diabetic Peripheral Neuropathy in a Mouse Induced by Streptozotocin>

It was found that xylitol is capable of preventing the peripheral neuropathy that is caused by streptozotocin-induced diabetes. Therefore, whether xylitol had a therapeutic action of alleviating peripheral neuropathy after the development of diabetic peripheral neuropathy was subsequently examined.

(1) Administration of Test Article

Six to seven-week-old male C57BL/6J mice were used for the tests as with Example 7. All the mice were acclimated for seven days after arrival. Then, the mice were grouped into the following four groups: a control group, a group receiving streptozotocin, a group receiving 1 mg/kg xylitol, and a group receiving streptozotocin+1 mg/kg xylitol. Each group consisted of nine mice.

On Day 8 of acclimation, the mice in the group receiving streptozotocin and the group receiving streptozotocin+1 mg/kg xylitol were administered with streptozotocin at a dose of 200 mg/kg. A massive dose of streptozotocin destroys pancreatic cells in the mice. As a result, insulin is no longer secreted, and thus, diabetes can be developed in the mice. This day was designated as the first day of the administration (Day 0). Note that streptozotocin was administered only on the first day of administration.

The mice in the group receiving 1 mg/kg xylitol were orally administered with xylitol at a dose of 1 mg/kg daily from Day 0. The mice in the group receiving streptozotocin+1 mg/kg xylitol were orally administered with xylitol at a dose of 1 mg/kg daily from Day 21 of administration.

(2) Cold Plate Test

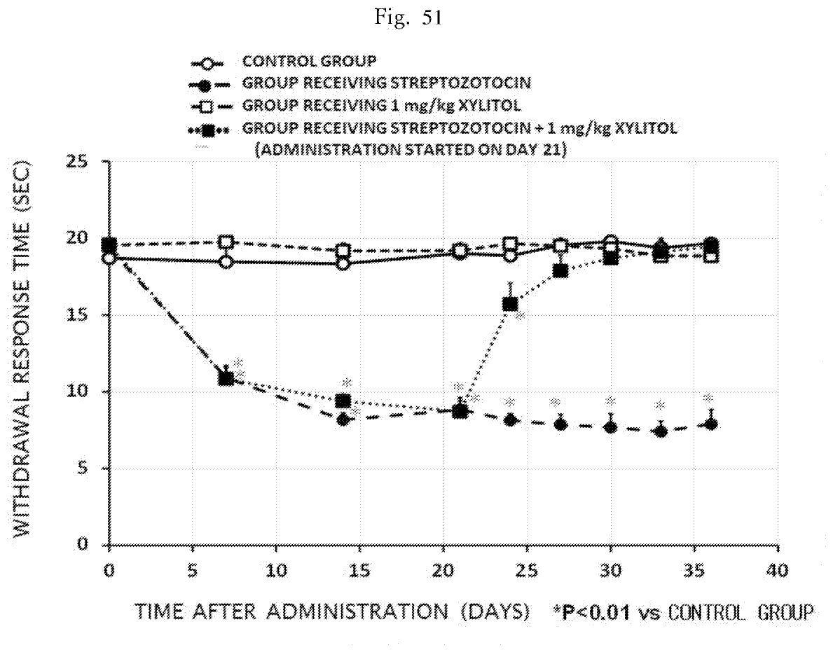

A cold plate test was performed to examine the effect due to xylitol on paresthesia that is caused by a low temperature stimulus. The mice in the four groups described in (1) of the present example (Example 8) were placed on a cold plate set at 4° C., and withdrawal response time (latent time) was measured. It is presumed that a shorter withdrawal response time (latent time) reflects a greater degree of shunning the low temperature stimulus by the cold plate. The results are shown in FIG. 51.

With reference to FIG. 51, the horizontal axis represents the time after administration (days), and the vertical axis represents the average value of the withdrawal response time (seconds) of the mice in each group. It can be determined that the shorter withdrawal response time reflects a greater degree of shunning the low temperature stimulus. The solid line with white circles represents the control group; the broken line with black circles represents the group receiving streptozotocin; the broken line with white squares represents the group receiving 1 mg/kg xylitol; and the dotted line with black squares represents the group receiving streptozotocin+1 mg/kg xylitol. Note that, in the group receiving streptozotocin+1 mg/kg xylitol, xylitol was administered from Day 21 from the start of the administration. A “*” mark was added to a measurement that was determined to have a significant difference from that of the control group in a test, using a significance level of 1% (in the figure, shown as “*P<0.01 vs control group”).

The control group (solid line with white circles) exhibited a stable withdrawal response time ranging from 18 seconds to 20 seconds throughout the experimental period. In the group receiving streptozotocin (broken line with black circles) and the group receiving streptozotocin+1 mg/kg xylitol (dotted line with black squares), the withdrawal response time was reduced from Day 7. On Day 14, in both the group receiving streptozotocin (broken line with black circles) and the group receiving streptozotocin+1 mg/kg xylitol (dotted line with black squares), the withdrawal response time (latent time) was greatly reduced.

However, in the group receiving streptozotocin+1 mg/kg xylitol (dotted line with black squares), the withdrawal response time was extended from Day 21 onwards, the day when the administration of xylitol was started. On Day 30, the withdrawal response time became similar to that of the control group (solid line with white circles).

On the other hand, the group receiving 1 mg/kg xylitol (broken line with white squares), which received only xylitol, exhibited a response time similar to that of the control group (solid line with white circles) throughout the experimental period, with no reduction in the withdrawal response time.

(3) Von Frey Test

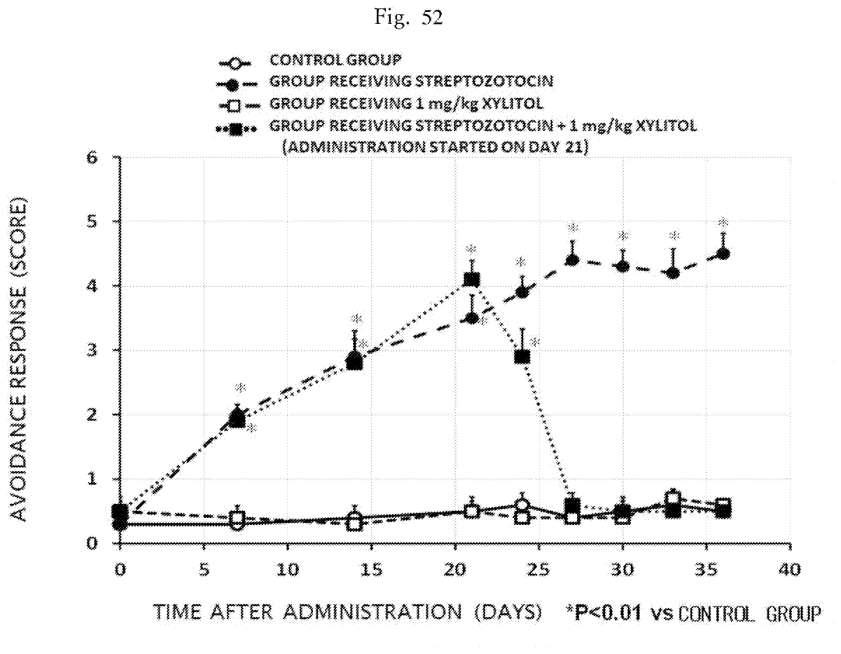

The mice in the four groups described in (1) of the present example (Example 8) were placed in a cage, and the avoidance response frequency (score) was measured by pressing a filament with a bending force of 0.16 g against the back of the hind paw. The results are shown in FIG. 52.

With reference to FIG. 52, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving streptozotocin; the broken line with white squares represents the group receiving 1 mg/kg xylitol; and the dotted line with black squares represents the group receiving streptozotocin+1 mg/kg xylitol. Note that, in the group receiving streptozotocin+1 mg/kg xylitol, xylitol was administered from Day 21 from the start of the administration. A “*” mark was added to a measurement that was determined to have a significant difference from that of the control group in a test, using a significance level of 1% (in the figure, shown as “*P<0.01 vs control group”).

The control group (solid line with white circles) exhibited a score of one or less stably throughout the experimental period. In the group receiving streptozotocin (broken line with black circles) and the group receiving streptozotocin+1 mg/kg xylitol (dotted line with black squares), the avoidance response score was increased from Day 7. However, in the group receiving streptozotocin+1 mg/kg xylitol (dotted line with black squares), the avoidance response was reduced from Day 21 onwards, the day when the administration of xylitol was started. On Day 27, the avoidance response was similar to that of the control group (solid line with white circles). Thus, xylitol was able to reduce the avoidance response even when xylitol was administered after the avoidance response score was increased.

The group receiving 1 mg/kg xylitol (broken line with white squares), which received xylitol, exhibited an avoidance response similar to that of the control group (solid line with white circles) throughout the experimental period.

Both the cold plate test (FIG. 51) and the von Frey test (FIG. 52) demonstrated that the group receiving streptozotocin (broken line with black circles) and the group receiving streptozotocin+1 mg/kg xylitol (dotted line with black squares) exhibited a significant change in both the avoidance response score and the withdrawal response time compared with the control group (solid line with white circles) up to Day 21, the change being a shift to a greater degree of shunning the stimulus. This demonstrates that the administration of streptozotocin resulted in the onset of diabetes in the mice, causing peripheral neuropathy (peripheral nerve hypersensitivity) to develop.

However, in the group receiving streptozotocin+1 mg/kg xylitol (dotted line with black squares), the responses in both the von Frey test and the cold plate test returned to a level similar to those of the control group (solid line with white circles) from Day 21 onwards, the day when the administration of xylitol was started. The group receiving 1 mg/kg xylitol (broken line with white squares), which received xylitol, exhibited responses similar to those of the control group (solid line with white circles) throughout the experimental period. Therefore, it can be concluded that xylitol treats the peripheral neuropathy (peripheral nerve hypersensitivity) induced by streptozotocin-induced diabetes. In other words, xylitol serves as a therapeutic composition (therapeutic agent) for peripheral neuropathy that is caused by diabetes.

### (Example 9) D-Threitol-Containing Preventive Agent for Diabetic Peripheral Neuropathy

### <Preventive Action of D-Threitol on Diabetic Peripheral Neuropathy in a Mouse Induced by Streptozotocin>

The preventive effect of D-threitol on hyperesthesia that occurs in diabetic peripheral neuropathy was examined. Examples of hyperesthesia include paresthesia that is caused by a low temperature stimulus and allodynia (severe pain induced by a tactile stimulus that does not usually cause pain) that is caused by a mechanical stimulus. D-threitol was orally administered as a test drug to a mouse simultaneously with the administration of streptozotocin, and the following tests (cold plate test and von Frey test) were performed.

(1) Administration of Test Drug

Six to seven-week-old male C57BL/6J mice were used for the tests as with Example 7. All the mice were acclimated for seven days after arrival. Then, the mice were grouped into the following four groups: a control group, a group receiving streptozotocin, a group receiving 5 mg/kg D-threitol, and a group receiving streptozotocin+5 mg/kg D-threitol. Each group consisted of five mice.

On Day 8 of acclimation, the mice in the group receiving streptozotocin and the group receiving streptozotocin+5 mg/kg D-threitol were administered with streptozotocin at a dose of 200 mg/kg. A massive dose of streptozotocin destroys pancreatic cells in the mice. As a result, insulin is no longer secreted, and thus, diabetes can be developed in the mice. This day was designated as the first day of the administration (Day 0). Note that streptozotocin was administered only on the first day of administration.

The mice in the group receiving 5 mg/kg D-threitol and the group receiving streptozotocin+5 mg/kg D-threitol were orally administered with D-threitol at a dose of 5 mg/kg daily from Day 0.

(2) Cold Plate Test

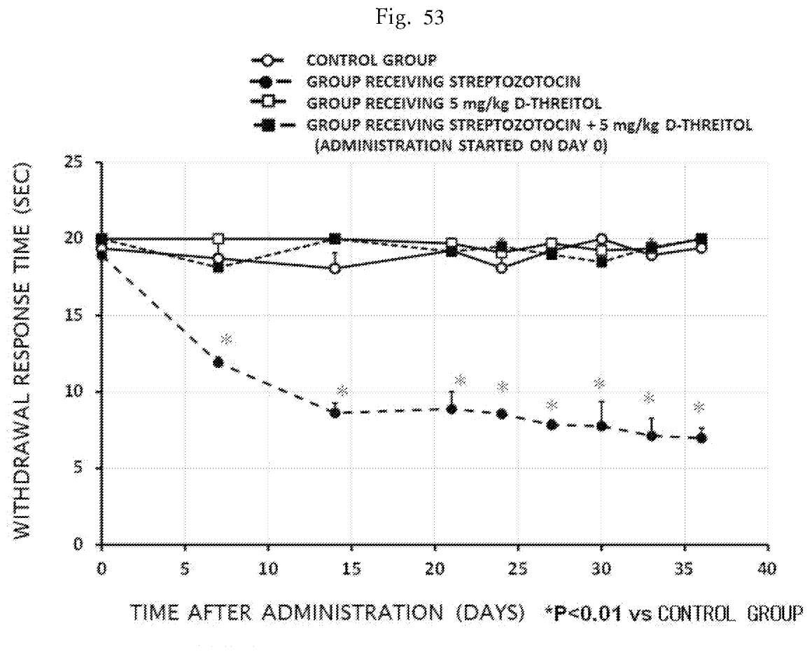

A cold plate test was performed to examine the effect of D-threitol on paresthesia that is caused by a low temperature stimulus. The mice in the four groups described in (1) of the present example (Example 9) were placed on a cold plate set at 4° C., and withdrawal response time (latent time) was measured. It is presumed that a shorter latent time reflects a greater degree of shunning the low temperature stimulus by the cold plate. The results are shown in FIG. 53.

With reference to FIG. 53, the horizontal axis represents the time after administration (days), and the vertical axis represents the average value of the withdrawal response time (seconds) of the mice in each group. The solid line with white circles represents the control group; the broken line with black circles represents the group receiving streptozotocin; the solid line with white squares represents the group receiving 5 mg/kg D-threitol; and the broken line with black squares represents the group receiving streptozotocin+5 mg/kg D-threitol. A “*” mark was added to a measurement that was determined to have a significant difference from that of the control group in a test, using a significance level of 1% (in the figure, shown as “*P<0.01 vs control group”).

The control group (solid line with white circles) exhibited a stable withdrawal response time ranging from 18 seconds to 20 seconds throughout the experimental period. In the group receiving streptozotocin (broken line with black circles), the withdrawal response time was reduced from Day 7. On Day 14, the withdrawal response time (latent time) was greatly reduced compared with the control group (solid line with white circles).

On the other hand, the group receiving 5 mg/kg D-threitol (solid line with white squares), which received only D-threitol, and the group receiving streptozotocin+5 mg/kg D-threitol (broken line with black squares), which received D-threitol in combination with streptozotocin, exhibited a response time similar to that of the control group (solid line with white circles) throughout the experimental period, with no reduction in the withdrawal response time. In these groups, the reduction in the latent time was suppressed compared with the group receiving streptozotocin (broken line with black circles).

(3) Von Frey Test

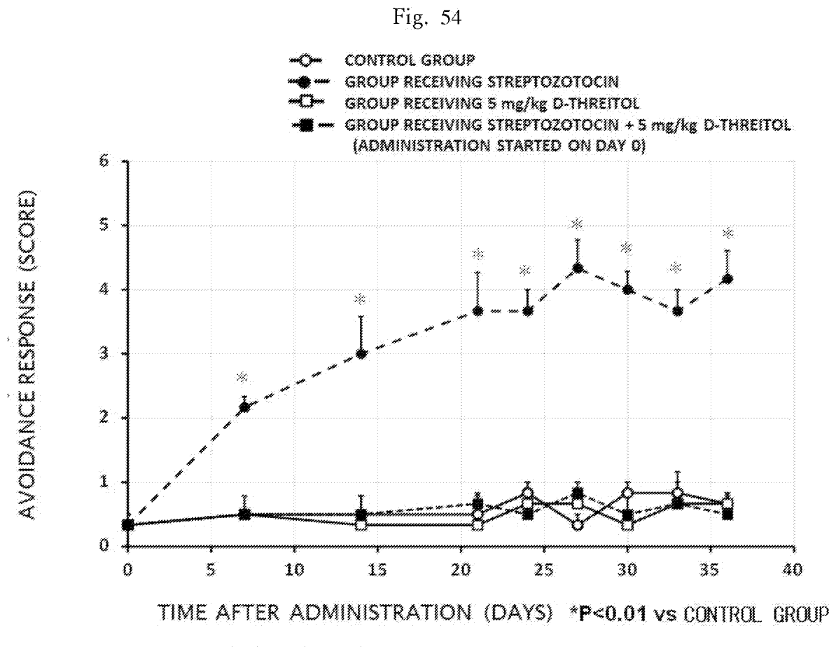

The mice in the four groups described in (1) of the present example (Example 9) were placed in a cage, and the avoidance response frequency (score) was measured by pressing a filament with a bending force of 0.16 g against the back of the hind paw. The results are shown in FIG. 54.

With reference to FIG. 54, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving streptozotocin; the solid line with white squares represents the group receiving 5 mg/kg D-threitol; and the broken line with black squares represents the group receiving streptozotocin+5 mg/kg D-threitol. A “*” mark was added to a measurement that was determined to have a significant difference from that of the control group in a test, using a significance level of 1% (in the figure, shown as “*P<0.01 vs control group”).

The control group (solid line with white circles) exhibited a stable score of one or less throughout the experimental period. In the group receiving streptozotocin (broken line with black circles), the avoidance response score was increased from Day 7. On the other hand, the group receiving 5 mg/kg D-threitol (solid line with white squares) and the group receiving streptozotocin+5 mg/kg D-threitol (broken line with black squares), which received D-threitol, exhibited avoidance response scores similar to that of the control group (solid line with white circles) throughout the experimental period. In these two groups, an increase in the avoidance response score was suppressed compared with the group receiving streptozotocin (broken line with black circles).

Both the cold plate test (FIG. 53) and the von Frey test (FIG. 54) demonstrated that the group receiving streptozotocin (broken line with black circles) exhibited a significant change in both the avoidance response score and the withdrawal response time compared with the control group (solid line with white circles), the change being a shift to a greater degree of shunning the stimulus. This demonstrates that the administration of streptozotocin resulted in the onset of diabetes in the mice, causing peripheral neuropathy (peripheral nerve hypersensitivity) to develop.

On the other hand, the group receiving 5 mg/kg D-threitol (solid line with white squares) and the group receiving streptozotocin+5 mg/kg D-threitol (broken line with black squares), which received D-threitol, showed results similar to those of the control group (solid line with white circles). Therefore, it can be concluded that D-threitol suppresses the peripheral neuropathy (peripheral nerve hypersensitivity) induced by streptozotocin-induced diabetes. In other words, D-threitol serves as a preventive composition (preventive agent) for peripheral neuropathy that is caused by diabetes.

### (Example 10) D-Threitol-Containing Therapeutic Agent for Diabetic Peripheral Neuropathy

### <Therapeutic Action of D-Threitol on Diabetic Peripheral Neuropathy in a Mouse Induced by Streptozotocin>

It was found that D-threitol is capable of preventing the peripheral neuropathy that is caused by streptozotocin-induced diabetes. Therefore, whether D-threitol had a therapeutic action of alleviating peripheral neuropathy after the development of diabetic peripheral neuropathy was subsequently examined.

(1) Administration of Test Article

Six to seven-week-old male C57BL/6J mice were used for the tests as with Example 7. All the mice were acclimated for seven days after arrival. Then, the mice were grouped into the following four groups: a control group, a group receiving streptozotocin, a group receiving 5 mg/kg D-threitol, and a group receiving streptozotocin+5 mg/kg D-threitol. Each group consisted of five mice.

On Day 8 of acclimation, the mice in the group receiving streptozotocin and the group receiving streptozotocin+5 mg/kg D-threitol were administered with streptozotocin at a dose of 200 mg/kg. A massive dose of streptozotocin destroys pancreatic cells in the mice. As a result, insulin is no longer secreted, and thus, diabetes can be developed in the mice. This day was designated as the first day of the administration (Day 0). Note that streptozotocin was administered only on the first day of administration.

The mice in the group receiving 5 mg/kg D-threitol were orally administered with D-threitol at a dose of 5 mg/kg daily from Day 0. The mice in the group receiving streptozotocin+5 mg/kg D-threitol were orally administered with D-threitol at a dose of 5 mg/kg daily from Day 21 of administration.

(2) Cold Plate Test

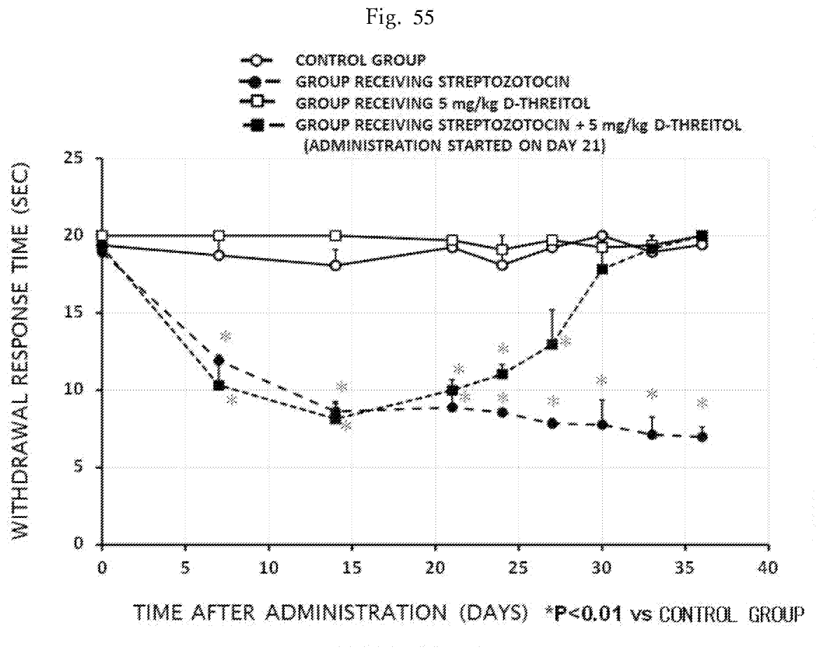

A cold plate test was performed to examine the effect of D-threitol on paresthesia that is caused by a low temperature stimulus. The mice in the four groups described in (1) of the present example (Example 10) were placed on a cold plate set at 4° C., and withdrawal response time (latent time) was measured. It is presumed that a shorter latent time reflects a greater degree of shunning the low temperature stimulus by the cold plate. The results are shown in FIG. 55.

With reference to FIG. 55, the horizontal axis represents the time after administration (days), and the vertical axis represents the average value of the withdrawal response time (seconds) of the mice in each group. It can be determined that the shorter withdrawal response time reflects a greater degree of shunning the low temperature stimulus. The solid line with white circles represents the control group; the broken line with black circles represents the group receiving streptozotocin; the solid line with white squares represents the group receiving 5 mg/kg D-threitol; and the broken line with black squares represents the group receiving streptozotocin+5 mg/kg D-threitol. Note that, in the group receiving streptozotocin+5 mg/kg D-threitol, D-threitol was administered from Day 21 from the start of the administration. A “*” mark was added to a measurement that was determined to have a significant difference from that of the control group in a test, using a significance level of 1% (in the figure, shown as “*P<0.01 vs control group”).

In the control group (solid line with white circles) the withdrawal response time was stable from 18 seconds to 20 seconds throughout the experimental period. In the group receiving streptozotocin (broken line with black circles) and the group receiving streptozotocin+5 mg/kg D-threitol (broken line with black squares), the withdrawal response time was reduced from Day 7. On Day 14, in both the group receiving streptozotocin (broken line with black circles) and the group receiving streptozotocin+5 mg/kg D-threitol (broken line with black squares), the withdrawal response time (latent time) was greatly reduced.

However, in the group receiving streptozotocin+5 mg/kg D-threitol (broken line with black squares), the withdrawal response time was extended from Day 21 onwards, the day when the administration of D-threitol was started. On Day 30, the withdrawal response time became similar to that of the control group.

On the other hand, the group receiving 5 mg/kg D-threitol (solid line with white squares), which received only D-threitol, exhibited a response time similar to that of the control group (solid line with white circles) throughout the experimental period, with no reduction in the withdrawal response time.

(3) Von Frey Test

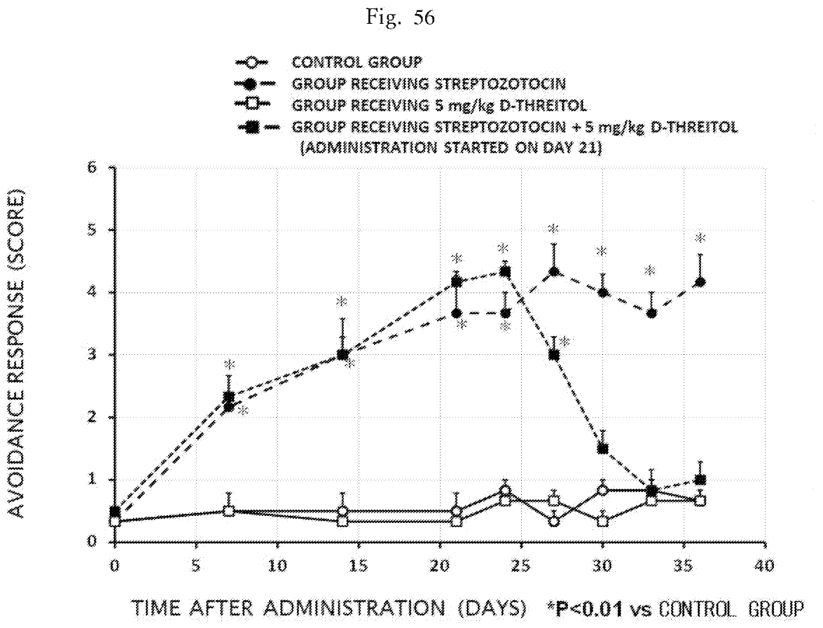

The mice in the four groups described in (1) of the present example (Example 10) were placed in a cage, and the avoidance response frequency (score) was measured by pressing a filament with a bending force of 0.16 g against the back of the hind paw. The results are shown in FIG. 56.

With reference to FIG. 56, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving streptozotocin; the solid line with white squares represents the group receiving 5 mg/kg D-threitol; and the broken line with black squares represents the group receiving streptozotocin+5 mg/kg D-threitol. Note that, in the group receiving streptozotocin+5 mg/kg D-threitol, D-threitol was administered from Day 21 from the start of the administration. A “*” mark was added to a measurement that was determined to have a significant difference from that of the control group in a test, using a significance level of 1% (in the figure, shown as “*P<0.01 vs control group”).

The control group (solid line with white circles) exhibited a score of one or less stably throughout the experimental period. In the group receiving streptozotocin (broken line with black circles) and the group receiving streptozotocin+5 mg/kg D-threitol (broken line with black squares), the avoidance response score was increased from Day 7. However, in the group receiving streptozotocin+5 mg/kg D-threitol (broken line with black squares), the avoidance response was reduced from Day 24 onwards, the day when the administration of D-threitol was started. On Day 33, the avoidance response was similar to that of the control group (solid line with white circles). Thus, D-threitol was able to reduce the avoidance response even when D-threitol was administered after the avoidance response score was increased.

The group receiving D-threitol (solid line with white squares), which received D-threitol, exhibited an avoidance response similar to that of the control group throughout the experimental period.

Both the cold plate test (FIG. 55) and the von Frey test (FIG. 56) demonstrated that the group receiving streptozotocin (broken line with black circles) and the group receiving streptozotocin+5 mg/kg D-threitol (broken line with black squares) exhibited a significant change in both the avoidance response score and the withdrawal response time compared with the control group (solid line with white circles) up to Day 21, the change being a shift to a greater degree of shunning the stimulus. This demonstrates that the administration of streptozotocin resulted in the onset of diabetes in the mice, causing peripheral neuropathy (peripheral nerve hypersensitivity) to develop.

However, in the group receiving streptozotocin+5 mg/kg D-threitol (broken line with black squares), the responses in both the von Frey test and the cold plate test returned to a level similar to those of the control group (solid line with white circles) from Day 21 onwards, the day when the administration of D-threitol was started. The group receiving 5 mg/kg D-threitol (solid line with white squares), which received D-threitol, exhibited responses similar to those of the control group (solid line with white circles) throughout the experimental period. Therefore, it can be concluded that D-threitol treats the peripheral neuropathy (peripheral nerve hypersensitivity) induced by streptozotocin-induced diabetes. In other words, D-threitol serves as a therapeutic composition (therapeutic agent) for peripheral neuropathy that is caused by diabetes.

### (Example 11) L-Talitol-Containing Preventive Agent for Diabetic Peripheral Neuropathy

### <Preventive Action of L-Talitol on Diabetic Peripheral Neuropathy in a Mouse Induced by Streptozotocin>

The preventive effect of L-talitol on hyperesthesia that occurs in diabetic peripheral neuropathy was examined. Examples of hyperesthesia include paresthesia that is caused by a low temperature stimulus and allodynia (severe pain induced by a tactile stimulus that does not usually cause pain) that is caused by a mechanical stimulus. L-talitol was orally administered as a test drug to a mouse simultaneously with the administration of streptozotocin, and the following tests (cold plate test and von Frey test) were performed.

(1) Administration of Test Drug

Six to seven-week-old male C57BL/6J mice were used for the tests as with Example 7. All the mice were acclimated for seven days after arrival. Then, the mice were grouped into the following four groups: a control group, a group receiving streptozotocin, a group receiving 5 mg/kg L-talitol, and a group receiving streptozotocin+5 mg/kg L-talitol. Each group consisted of five mice.

On Day 8 of acclimation, the mice in the group receiving streptozotocin and the group receiving streptozotocin+5 mg/kg L-talitol were administered with streptozotocin at a dose of 200 mg/kg. A massive dose of streptozotocin destroys pancreatic cells in the mice. As a result, insulin is no longer secreted, and thus, diabetes can be developed in the mice. This day was designated as the first day of the administration (Day 0). Note that streptozotocin was administered only on the first day of administration.

The mice in the group receiving 5 mg/kg L-talitol and the group receiving streptozotocin+5 mg/kg L-talitol were orally administered with L-talitol at a dose of 5 mg/kg daily from Day 0.

(2) Cold Plate Test

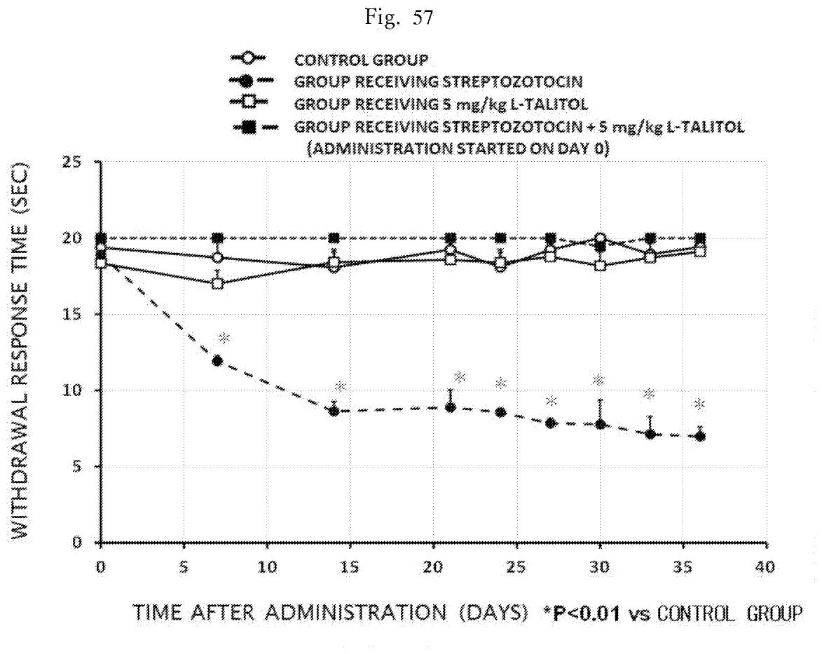

A cold plate test was performed to examine the effect of L-talitol on paresthesia that is caused by a low temperature stimulus. The mice in the four groups described in (1) of the present example (Example 11) were placed on a cold plate set at 4° C., and withdrawal response time (latent time) was measured. It is presumed that a shorter latent time reflects a greater degree of shunning the low temperature stimulus by the cold plate. The results are shown in FIG. 57.

With reference to FIG. 57, the horizontal axis represents the time after administration (days), and the vertical axis represents the average value of the withdrawal response time (seconds) of the mice in each group. The solid line with white circles represents the control group; the broken line with black circles represents the group receiving streptozotocin; the solid line with white squares represents the group receiving 5 mg/kg L-talitol; and the broken line with black squares represents the group receiving streptozotocin+5 mg/kg L-talitol. A “*” mark was added to a measurement that was determined to have a significant difference from that of the control group in a test, using a significance level of 1% (in the figure, shown as “*P<0.01 vs control group”).

The control group (solid line with white circles) exhibited a stable withdrawal response time ranging from 18 seconds to 20 seconds throughout the experimental period. In the group receiving streptozotocin (broken line with black circles), the withdrawal response time was reduced from Day 7. On Day 14, the withdrawal response time (latent time) was greatly reduced compared with the control group (solid line with white circles).

On the other hand, the group receiving 5 mg/kg L-talitol (solid line with white squares), which received only L-talitol, and the group receiving streptozotocin+5 mg/kg L-talitol (broken line with black squares), which received L-talitol in combination with streptozotocin, exhibited a response time similar to that of the control group (solid line with white circles) throughout the experimental period, with no reduction in the withdrawal response time. In these groups, the reduction in the withdrawal response time (latent time) was suppressed compared with the group receiving streptozotocin (broken line with black circles).

(3) Von Frey Test

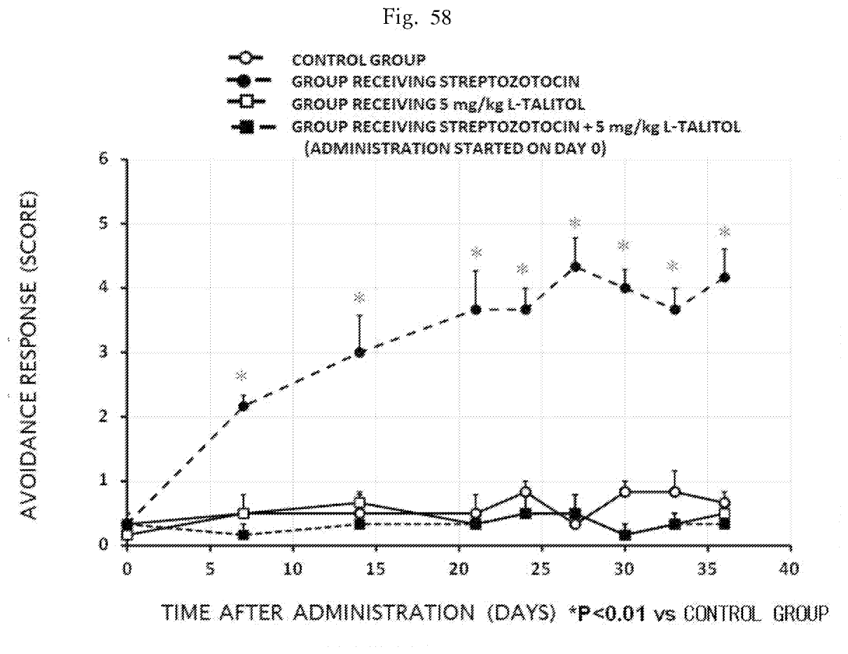

The mice in the four groups described in (1) of the present example (Example 11) were placed in a cage, and the avoidance response frequency (score) was measured by pressing a filament with a bending force of 0.16 g against the back of the hind paw. The results are shown in FIG. 58.

With reference to FIG. 58, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving streptozotocin; the solid line with white squares represents the group receiving 5 mg/kg L-talitol; and the broken line with black squares represents the group receiving streptozotocin+5 mg/kg L-talitol. A “*” mark was added to a measurement that was determined to have a significant difference from that of the control group in a test, using a significance level of 1% (in the figure, shown as “*P<0.01 vs control group”).

The control group (solid line with white circles) exhibited a stable score of one or less throughout the experimental period. In the group receiving streptozotocin (broken line with black circles), the avoidance response score was increased from Day 7. On the other hand, the group receiving 5 mg/kg L-talitol (solid line with white squares) and the group receiving streptozotocin+5 mg/kg L-talitol (broken line with black squares), which received L-talitol, exhibited avoidance response scores similar to that of the control group (solid line with white circles) throughout the experimental period. In these two groups, an increase in the avoidance response score was suppressed compared with the group receiving streptozotocin (broken line with black circles).

Both the cold plate test (FIG. 57) and the von Frey test (FIG. 58) demonstrated that the group receiving streptozotocin (broken line with black circles) exhibited a significant change in both the avoidance response score and the withdrawal response time compared with the control group (solid line with white circles), the change being a shift to a greater degree of shunning the stimulus. This demonstrates that the administration of streptozotocin resulted in the onset of diabetes in the mice, causing peripheral neuropathy (peripheral nerve hypersensitivity) to develop.

On the other hand, the group receiving 5 mg/kg L-talitol (solid line with white squares) and the group receiving streptozotocin+5 mg/kg L-talitol (broken line with black squares), which received L-talitol, showed results similar to those of the control group (solid line with white circles). Therefore, it can be concluded that L-talitol suppresses the peripheral neuropathy (peripheral nerve hypersensitivity) induced by streptozotocin-induced diabetes. In other words, L-talitol serves as a preventive composition (preventive agent) for peripheral neuropathy that is caused by diabetes.

### (Example 12) L-Talitol-Containing Therapeutic Agent for Diabetic Peripheral Neuropathy

### <Therapeutic Action of L-Talitol on Diabetic Peripheral Neuropathy in a Mouse Induced by Streptozotocin>

It was found that L-talitol is capable of preventing the peripheral neuropathy that is caused by streptozotocin-induced diabetes. Therefore, whether L-talitol had a therapeutic action of alleviating peripheral neuropathy after the development of diabetic peripheral neuropathy was subsequently examined.

(1) Administration of Test Article

Six to seven-week-old male C57BL/6J mice were used for the tests as with Example 7. All the mice were acclimated for seven days after arrival. Then, the mice were grouped into the following four groups: a control group, a group receiving streptozotocin, a group receiving 5 mg/kg L-talitol, and a group receiving streptozotocin+5 mg/kg L-talitol. Each group consisted of five mice.

On Day 8 of acclimation, the mice in the group receiving streptozotocin and the group receiving streptozotocin+5 mg/kg L-talitol were administered with streptozotocin at a dose of 200 mg/kg. A massive dose of streptozotocin destroys pancreatic cells in the mice. As a result, insulin is no longer secreted, and thus, diabetes can be developed in the mice. This day was designated as the first day of the administration (Day 0). Note that streptozotocin was administered only on the first day of administration.

The mice in the group receiving 5 mg/kg L-talitol were orally administered with L-talitol at a dose of 5 mg/kg daily from Day 0. The mice in the group receiving streptozotocin+5 mg/kg L-talitol were orally administered with L-talitol at a dose of 5 mg/kg daily from Day 21 of administration.

(2) Cold Plate Test

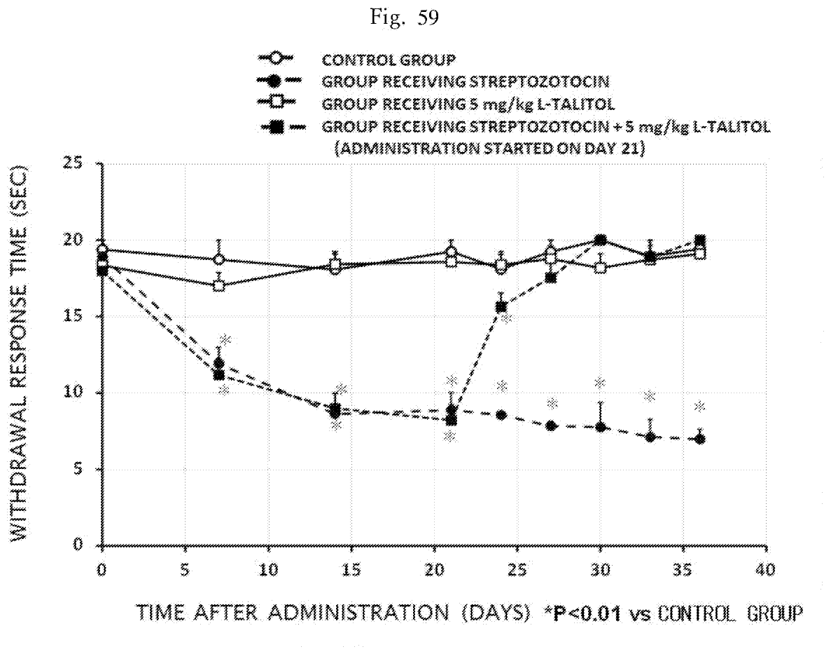

A cold plate test was performed to examine the effect of L-talitol on paresthesia that is caused by a low temperature stimulus. The mice in the four groups described in (1) of the present example (Example 12) were placed on a cold plate set at 4° C., and withdrawal response time (latent time) was measured. It is presumed that a shorter latent time reflects a greater degree of shunning the low temperature stimulus by the cold plate. The results are shown in FIG. 59.

With reference to FIG. 59, the horizontal axis represents the time after administration (days), and the vertical axis represents the average value of the withdrawal response time (seconds) of the mice in each group. It can be determined that the shorter withdrawal response time reflects a greater degree of shunning the low temperature stimulus. The solid line with white circles represents the control group; the broken line with black circles represents the group receiving streptozotocin; the solid line with white squares represents the group receiving 5 mg/kg L-talitol; and the broken line with black squares represents the group receiving streptozotocin+5 mg/kg L-talitol. Note that, in the group receiving streptozotocin+5 mg/kg L-talitol, L-talitol was administered from Day 21 from the start of the administration. A “*” mark was added to a measurement that was determined to have a significant difference from that of the control group in a test, using a significance level of 1% (in the figure, shown as “*P<0.01 vs control group”).

The control group (solid line with white circles) exhibited a stable withdrawal response time ranging from 18 seconds to 20 seconds throughout the experimental period. In the group receiving streptozotocin (broken line with black circles) and the group receiving streptozotocin+5 mg/kg L-talitol (broken line with black squares), the withdrawal response time was reduced from Day 7. On Day 14, in both the group receiving streptozotocin (broken line with black circles) and the group receiving streptozotocin+5 mg/kg L-talitol (broken line with black squares), the latent time was greatly reduced.

However, in the group receiving streptozotocin+5 mg/kg L-talitol (broken line with black squares), the withdrawal response time was extended from Day 21 onwards, the day when the administration of L-talitol was started. On Day 27, the withdrawal response time became similar to that of the control group (solid line with white circles).

On the other hand, the group receiving 5 mg/kg L-talitol (solid line with white squares), which received only L-talitol, exhibited a response time similar to that of the control group (solid line with white circles) throughout the experimental period, with no reduction in the withdrawal response time.

(3) Von Frey Test

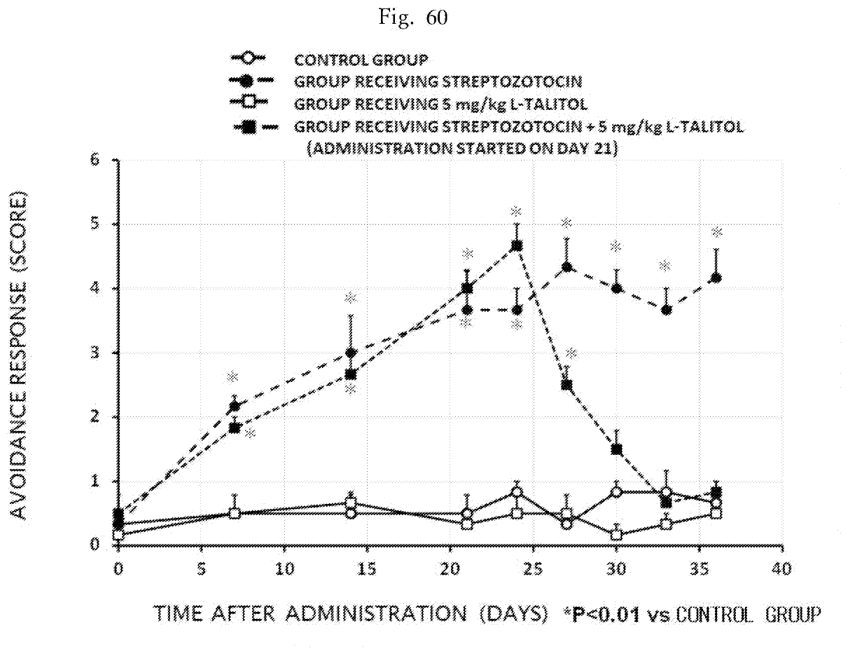

The mice in the four groups described in (1) of the present example (Example 12) were placed in a cage, and the avoidance response frequency (score) was measured by pressing a filament with a bending force of 0.16 g against the back of the hind paw. The results are shown in FIG. 60.

With reference to FIG. 60, the horizontal axis represents the time after administration (days), and the vertical axis represents the average value of the avoidance responses (score) of the mice in each group. It is presumed that more frequent avoidance responses reflect a greater degree of shunning the stimulus provided by the filament. The solid line with white circles represents the control group; the broken line with black circles represents the group receiving streptozotocin; the solid line with white squares represents the group receiving 5 mg/kg L-talitol; and the broken line with black squares represents the group receiving streptozotocin+5 mg/kg L-talitol. Note that, in the group receiving streptozotocin+5 mg/kg L-talitol, L-talitol was administered from Day 21 from the start of the administration. A “*” mark was added to a measurement that was determined to have a significant difference from that of the control group in a test, using a significance level of 1% (in the figure, shown as “*P<0.01 vs control group”).

The control group (solid line with white circles) exhibited a score of one or less stably throughout the experimental period. In the group receiving streptozotocin (broken line with black circles) and the group receiving streptozotocin+5 mg/kg L-talitol (broken line with black squares), the avoidance response score was increased from Day 7. However, in the group receiving streptozotocin+5 mg/kg L-talitol (broken line with black squares), the avoidance response was reduced from Day 21 onwards, the day when the administration of L-talitol was started. On Day 33, the avoidance response was similar to that of the control group (solid line with white circles). Thus, L-talitol was able to reduce the avoidance response even when L-talitol was administered after the avoidance response score was increased.

The group receiving 5 mg/kg L-talitol (solid line with white squares), which received L-talitol, exhibited an avoidance response similar to that of the control group (solid line with white circles) throughout the experimental period.

Both the cold plate test (FIG. 59) and the von Frey test (FIG. 60) demonstrated that the group receiving streptozotocin (broken line with black circles) and the group receiving streptozotocin+5 mg/kg L-talitol (broken line with black squares) exhibited a significant change in both the avoidance response score and the withdrawal response time compared with the control group (solid line with white circles) up to Day 21, the change being a shift to a greater degree of shunning the stimulus. This demonstrates that the administration of streptozotocin resulted in the onset of diabetes in the mice, causing peripheral neuropathy (peripheral nerve hypersensitivity) to develop.

However, in the group receiving streptozotocin+5 mg/kg L-talitol (broken line with black squares), the responses in both the von Frey test and the cold plate test returned to a level similar to those of the control group (solid line with white circles) from Day 21 onwards, the day when the administration of L-talitol was started. The group receiving 5 mg/kg L-talitol (solid line with white squares), which received L-talitol, exhibited responses similar to those of the control group (solid line with white circles) throughout the experimental period. Therefore, it can be concluded that L-talitol treats the peripheral neuropathy (peripheral nerve hypersensitivity) induced by streptozotocin-induced diabetes. In other words, L-talitol serves as a therapeutic composition (therapeutic agent) for peripheral neuropathy that is caused by diabetes.

### (Example 13) Xylitol-Containing Peripheral Nerve Outgrowth Inhibition Suppressant

### <Suppression Effect Due to Xylitol on Nerve Outgrowth Inhibition of Peripheral Nerves Caused by Anticancer Drug>

It is generally known that neurite outgrowth is inhibited in peripheral neuropathy that is caused by anticancer drugs. Thus, a test is performed in order to investigate whether xylitol has an effect of suppressing the nerve outgrowth inhibition of the peripheral nerves that is caused by anticancer drugs. The suppression effect on the nerve outgrowth inhibition of the peripheral nerves was examined using a cell line PC-12 derived from rat pheochromocytoma (obtained from JCRB Cell Bank, National Institutes of Biomedical Innovation, Health and Nutrition).

(1) Cell Culture (Pre-Culture)

PC-12 cells were put in RPMI 1640 medium (manufactured by Sigma-Aldrich) containing 50 ng/mL NGF and 2% fetal bovine serum (FBS), suspended in the medium at 3×10⁵ cells/mL, and seeded in a 24-well plate at 1 mL each, followed by culturing for 4 days.

(2) Cell Culture (Main Culture)

After culturing, the medium was removed, and 2 mL of RPMI 1640 medium containing 2% fetal bovine serum (FBS), newly supplemented with one of formulations 1 to 21 listed in Table 1, was added to the cells, followed by culturing for 24 hours.

TABLE 1

| | | Presence or | Amount | Amount of |
|---|---|---|---|---|
| | | Absence of NGF | of Xylitol | Anticancer Agent |
| | | (50 ng/mL) | (μg/mL) | (μg/mL) |
| Formulation 1 | Vehicle | — | — | — |
| Formulation 2 | NGF | ○ | — | — |
| Formulation 3 | Xylitol | ○ | 0.1 | — |
| Formulation 4 | Xylitol | ○ | 1 | — |
| Formulation 5 | Xylitol | ○ | 10 | — |
| Formulation 6 | Oxaliplatin | ○ | — | 0.4 |
| Formulation 7 | Oxaliplatin + Xylitol | ○ | 0.1 | 0.4 |
| Formulation 8 | Oxaliplatin + Xylitol | ○ | 1 | 0.4 |
| Formulation 9 | Oxaliplatin + Xylitol | ○ | 10 | 0.4 |
| Formulation 10 | Paclitaxel | ○ | — | 0.0085 |
| Formulation 11 | Paclitaxel + Xylitol | ○ | 0.1 | 0.0085 |
| Formulation 12 | Paclitaxel + Xylitol | ○ | 1 | 0.0085 |
| Formulation 13 | Paclitaxel + Xylitol | ○ | 10 | 0.0085 |
| Formulation 14 | Vincristine | ○ | — | 0.0082 |
| Formulation 15 | Vincristine + Xylitol | ○ | 0.1 | 0.0082 |
| Formulation 16 | Vincristine + Xylitol | ○ | 1 | 0.0082 |
| Formulation 17 | Vincristine + Xylitol | ○ | 10 | 0.0082 |
| Formulation 18 | Bortezomib | ○ | — | 0.0019 |
| Formulation 19 | Bortezomib + Xylitol | ○ | 0.1 | 0.0019 |
| Formulation 20 | Bortezomib + Xylitol | ○ | 1 | 0.0019 |
| Formulation 21 | Bortezomib + Xylitol | ○ | 10 | 0.0019 |

※NGF: Nerve Growth Factor- β (NGF- β/β -NGF), Human. Recombinant (Manufactured by FUJIFILM Wako Pure Chemical Corporation)

(3) Neurite Evaluation and Cell Evaluation (Viability)

After culturing for 6 hours, the state of the cells was 5 photographed using an optical microscope, and the length of neurites was measured using image processing software “Imagej 1.50i” for comparison. Furthermore, the cell viability of the cells cultured for 24 hours was calculated using Cell Counting Kit-8 (Dojindo Laboratories) according to the protocol of the kit.

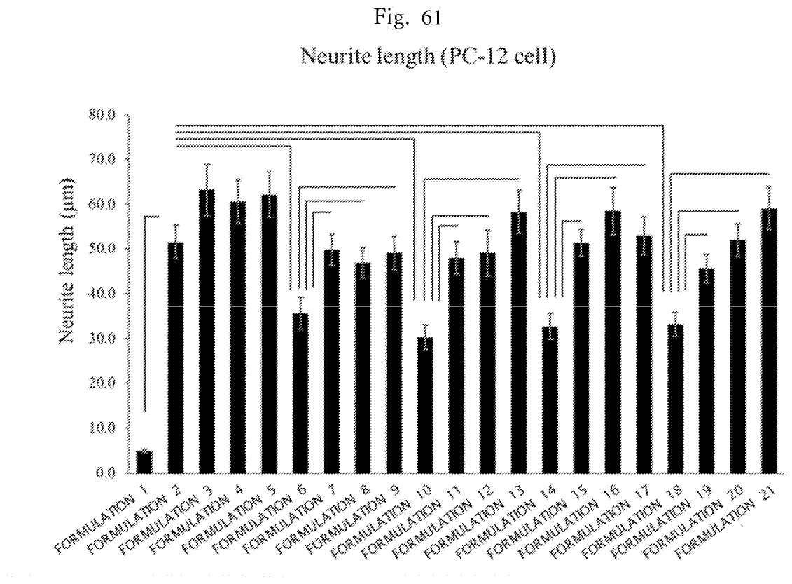

Referring to FIG. 61, the horizontal axis represents the formulation number, and the vertical axis represents the nerve length (μm). Neurite outgrowth can be confirmed by an increase in the nerve length. Neurite outgrowth was observed with the formulation 2 in which Nerve Growth Factor-β is supplemented, as compared with the formulation 1, which has no supplement.

It is found that, in the formulation 6, 10, 14, or 18, in which the anticancer drug oxaliplatin, paclitaxel, vincristine, or bortezomib is added to the formulation 2, the neurite outgrowth is inhibited as compared with the formulation 2.

It is found that, in the formulation 7, 8, 9, 11, 12, 13, 15, 16, 17, 19, 20, or 21, in which xylitol is further added to the formulation 6, 10, 14, or 18, inhibition of the neurite outgrowth caused by the anticancer drugs is suppressed.

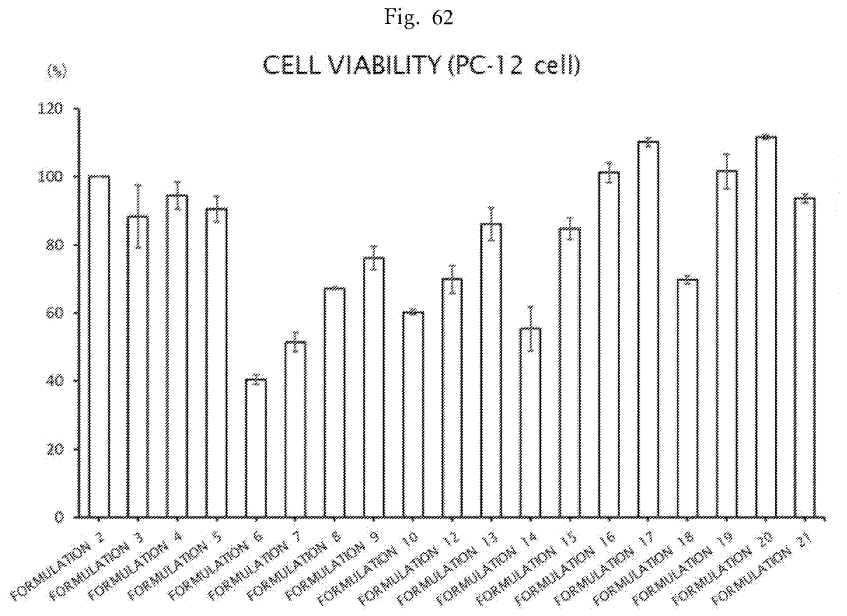

Referring to FIG. 62, the horizontal axis represents the formulation number, and the vertical axis represents the cell viability (%). A low cell viability value indicates cytotoxicity. Having set the cell viability with the formulation 2 as 100%, the cell viability with other formulations was calculated.

It is found that, in the formulation 6, 10, 14, or 18, in which the anticancer drug oxaliplatin, paclitaxel, vincristine, or bortezomib is added to the formulation 2, a value of the cell viability is lower than that of the formulation 2.

It is found that, in the formulation 7, 8, 9, 12, 13, 15, 16, 17, 19, 20, or 21, in which xylitol is further added to the formulation 6, 10, 14, or 18, each cell viability is improved as compared with the formulation 6, 10, 14 or 18, indicating that cytotoxicity caused by the anticancer drugs is suppressed.

### (Example 14) Xylitol-Containing Peripheral Nerve Outgrowth Inhibition Suppressant

### <Suppression Effect Due to Xylitol on Nerve Outgrowth Inhibition of Peripheral Nerves Caused by Anticancer Drugs>

As in Example 13, a test was performed in order to investigate whether xylitol has the effect of suppressing the nerve outgrowth inhibition of the peripheral nerves that is caused by anticancer drugs. The suppression effect on the nerve outgrowth inhibition of the peripheral nerves was examined using a human neuroblastoma cell line SH-SY5Y (obtained from KAC Co., Ltd.).

(1) Cell Culture (Pre-Culture)

After diluting 10 μL of 0.5 mg/mL iMatrix-511 silk (manufactured by Nippi, Inc.) in 2 mL of PBS, the resulting solution was added to a 24-well plate at 500 μL per well to perform immobilization at 4° C. overnight. After the immobilization, the iMatrix-511 silk solution was removed from the 24-well plate and the plate was washed with PBS. Afterwards, SH-SY5Y cells suspended in Ham's F-12K medium (manufactured by Fujifilm Wako Pure Chemical Corp.) containing 2% FBS at 3×10⁵ cells/mL were seeded in the 24-well plate at 1 mL each, followed by culturing for 4 days.

(2) Cell Culture (Main Culture)

After culturing, the medium was removed, and 2 mL of Ham's F-12K medium containing 2% FBS, newly supplemented with one of the formulations 23 to 42 listed in Table 2, was added to the cells, followed by culturing for 24 hours.

TABLE 2

| | | Amount | Amount of |
|---|---|---|---|
| | | of Xylitol | Anticancer Agent |
| | | (μg/mL) | (μg/mL) |
| Formulation 23 | Vehicle | — | — |
| Formulation 24 | Xylitol | 0.1 | — |
| Formulation 25 | Xylitol | 1 | — |
| Formulation 26 | Xylitol | 10 | — |
| Formulation 27 | Oxaliplatin | — | 0.4 |
| Formulation 28 | Oxaliplatin + Xylitol | 0.1 | 0.4 |
| Formulation 29 | Oxaliplatin + Xylitol | 1 | 0.4 |
| Formulation 30 | Oxaliplatin + Xylitol | 10 | 0.4 |
| Formulation 31 | Paclitaxel | — | 0.0085 |
| Formulation 32 | Paclitaxel + Xylitol | 0.1 | 0.0085 |
| Formulation 33 | Paclitaxel + Xylitol | 1 | 0.0085 |
| Formulation 34 | Paclitaxel + Xylitol | 10 | 0.0085 |
| Formulation 35 | Vincristine | — | 0.0082 |
| Formulation 36 | Vincristine + Xylitol | 0.1 | 0.0082 |
| Formulation 37 | Vincristine + Xylitol | 1 | 0.0082 |
| Formulation 38 | Vincristine + Xylitol | 10 | 0.0082 |
| Formulation 39 | Bortezomib | — | 0.0019 |
| Formulation 40 | Bortezomib + Xylitol | 0.1 | 0.0019 |
| Formulation 41 | Bortezomib + Xylitol | 1 | 0.0019 |
| Formulation 42 | Bortezomib + Xylitol | 10 | 0.0019 |

(3) Neurite Evaluation and Cell Evaluation (Viability)

After culturing for 6 hours, the state of the cells was photographed using an optical microscope, and the length of the neurites was measured using image processing software “Imagej 1.50i” for comparison. Furthermore, the cell viability of the cells cultured for 24 hours was calculated using Cell Counting Kit-8 (Dojindo Laboratories) according to the protocol of Cell Counting Kit-8 (https://www.dojindo.co.jp/technical/protocol/p01.pdf).

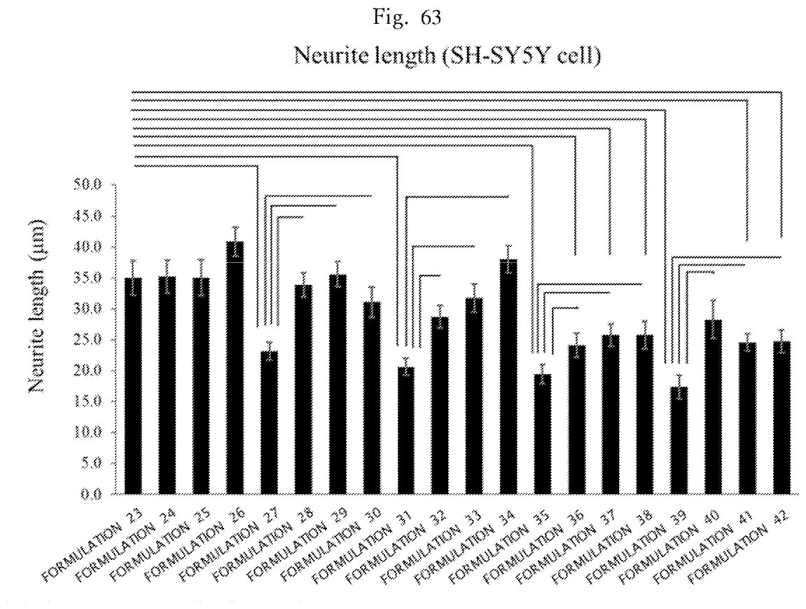

Referring to FIG. 63, the horizontal axis represents the formulation number, and the vertical axis represents the nerve length (μm). Neurite outgrowth can be confirmed by an increase in the nerve length. Neurite outgrowth was confirmed with the formulation 23 in which Nerve Growth Factor-β was supplemented.

It is found that, in the formulation 27, 31, 35, or 39, in which the anticancer drug oxaliplatin, paclitaxel, vincristine, or bortezomib is added to the formulation 23, the neurite outgrowth is inhibited as compared with the formulation 23.

It is found that, in the formulation 28, 29, 30, 32, 33, 34, 36, 37, 38, 40, 41, or 42, in which xylitol is further added to the formulation 27, 31, 35, or 39, inhibition of the neurite outgrowth caused by the anticancer drugs is suppressed.

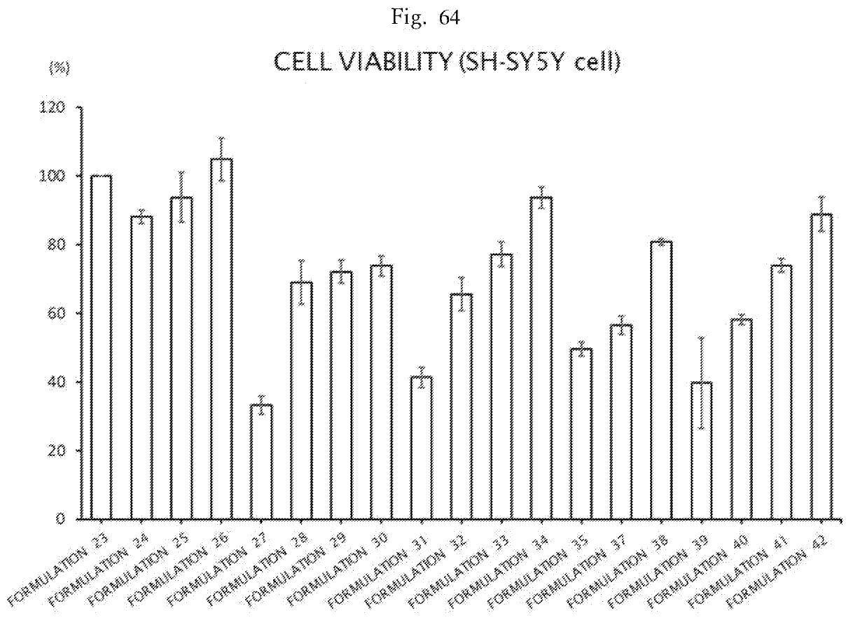

Referring to FIG. 64, the horizontal axis represents the formulation number, and the vertical axis represents the cell viability (%). A low cell viability value indicates cytotoxicity. Having set the cell viability with the formulation 23 as 100%, the cell viability with other formulations was calculated.

It is found that, in the formulation 27, 31, 35, or 39, in which the anticancer drug oxaliplatin, paclitaxel, vincristine, or bortezomib is added to the formulation 23, a value of the cell viability is lower than that of the formulation 23.

It is found that, in the formulation 28, 29, 30, 32, 33, 34, 37, 38, 40, 41, or 42, in which xylitol is further added to the formulation 27, 31, 35, or 39, each cell viability is improved as compared with the formulation 27, 31, 35, or 39, indicating that cytotoxicity caused by the anticancer drugs was suppressed.

As described above, the preventive or ameliorative agent according to the present invention was able to effectively suppress peripheral neuropathy that was caused by diabetes and anticancer drugs with various mechanisms of action. The ability to prevent or ameliorate peripheral neuropathy regardless of its causes makes it reasonable to conclude that the preventive or ameliorative agent according to the present invention ameliorates a possible primary cause of peripheral neuropathy such as axonal degeneration of the nerve cells or direct damage to the nerve cells. That is, it is presumed that the preventive or ameliorative agent according to the present invention can be effective for peripheral neuropathy that is caused by causes other than those described in the above-mentioned examples.

### INDUSTRIAL APPLICABILITY

The preventive or ameliorative agent according to the present invention can be used for ameliorating or preventing peripheral neuropathy. The agent of the present invention can also be used for treating peripheral neuropathy. In particular, the agent of the present invention can be suitably used for reducing, alleviating, or preventing not only peripheral neuropathy that is caused by administration of a DNA replication inhibitor (a platinum-containing drug (e.g., oxaliplatin) and/or an alkylating agent), a microtubule polymerization stabilizer, a microtubule polymerization inhibitor, a proteasome inhibitor, or the like, and peripheral neuropathy as a complication of diabetes, but also any other peripheral neuropathy that is caused by other reasons.

1. An agent for preventing or ameliorating peripheral neuropathy characterized by comprising at least one selected from xylitol, L-talitol, and D-threitol as an active ingredient.

2. The agent for preventing or ameliorating peripheral neuropathy according to claim 1, wherein the peripheral neuropathy is induced by administration of an anticancer drug.

3. The agent for preventing or ameliorating peripheral neuropathy according to claim 2, wherein the anticancer drug is a platinum-based anticancer drug, a microtubule polymerization stabilizer, a microtubule polymerization inhibitor, a proteasome inhibitor.

4. The agent for preventing or ameliorating peripheral neuropathy according to claim 1, in which the peripheral neuropathy is diabetic peripheral neuropathy.

5. The agent for preventing or ameliorating peripheral neuropathy according to claim 1, wherein the agent is a pharmaceutical product.

6. The agent for preventing or ameliorating peripheral neuropathy according to claim 1, wherein the agent is a food product.

7. The agent for preventing or ameliorating peripheral neuropathy according to claim 2, wherein the agent is a pharmaceutical product.

8. The agent for preventing or ameliorating peripheral neuropathy according to claim 3, wherein the agent is a pharmaceutical product.

9. The agent for preventing or ameliorating peripheral neuropathy according to claim 4, wherein the agent is a pharmaceutical product.

10. The agent for preventing or ameliorating peripheral neuropathy according to claim 2, wherein the agent is a food product.

11. The agent for preventing or ameliorating peripheral neuropathy according to claim 3, wherein the agent is a food product.

12. The agent for preventing or ameliorating peripheral neuropathy according to claim 4, wherein the agent is a food product.
